SEQUENCE LISTING (8 sequences)

SEQ ID NO: 1 - DNA, 301 bp, Homo sapiens
  misc_feature (147)..(171): mononucleotide repeats
gccatcatggaggatgacgagttggccctagacttagaagacttgctgagcttttcttaccaggtggcaaagggcatggctttcctcgcctccaagaatgtaagtgggagtgattctctaaagagttttgtgttttgtttttttgatttttttttttttttttttttttttgagaacagagcattttagagccatagttaaaatgcagaatgtcattttgaagtgtggtaaccaaaagcagaggaaatttagtttcttcatgttccaactgctgtctctttggaattcctgttctaattta

SEQ ID NO: 2 - DNA, 401 bp, Homo sapiens
  misc_feature (222)..(248): mononucleotide repeats
gttttttaaaatctttagaactggatccagtggtatagaaatcttcgatttttaaattcttaattttaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcaggtaaaaaaaaaaaaaaaaaaaaaaaaaaagggttaaaaatgttgaatggttaaaaaatgttttcattgacatatactgaagaagcttataaaggagctaaaatattttgaaatattattatacttggattagataactagctttaaatggctgtatttttctctcccctcctccactccact

SEQ ID NO: 3 - DNA, 301 bp, Homo sapiens
  misc_feature (188)..(209): mononucleotide repeats
ccctttctaaatgcgtattcgtgtaaatatattgggagagagctttgaattagaacgtccttttccgaaataggaaccactgctactctctaaaaaaggcaagcagataaaagagaacacgaaaaatattcctactccgcattcacactttctggtcactcgcgtttacaaacaagaaaagtgttgctaaaaaaaaaaaaaaaaaaaaaggccaggggagacatacatttaaatataaaaatagaactgtgccagcgactccggctggaattctgctgaaagggatgtgtcttcagaaacc

SEQ ID NO: 4 - DNA, 291 bp, Homo sapiens
  misc_feature (151)..(172): mononucleotide repeats
tctccaaagttgatctgattgtaaatattaaactgacatctttatgttgcaggtaaaggacctggataatcgaggcttgtcaaggacataaatgtcacgtccagctctgatatgcttcgcactgagcacatcacatttaggacgttgaagatttttttttttttttttttttaatatgcagtttgtaagaacaaaactggatggcatcagaattgtctggaagttttgtcttgggcagtatgggctgggccaaatgaaatgatttttataattctaaacaggttaccaaat

SEQ ID NO: 5 - DNA, 301 bp, Homo sapiens
caaatgaccccttcctgcccatcactgccttcctcaagacctaaaatagctccctatttagtgaaaaattatctgaatatttaaggtctgccttaacgtgatccccattgctgaattttacctcctgactccaaaaactcttctcttccctgggcccagtcctatttttttttttttttttttttttgtgagacagagtctcactctgtcacccaggttggaatgcaatggcacaatctccgctcactgcaagctccgcctcccgggttcacgccattctcctgcctcagcctcccgaata

SEQ ID NO: 6 - DNA, 601 bp, Homo sapiens
  misc_feature (299)..(327): mononucleotide repeats
ttaaaaagcaaaaaattggctgggcacagtggctcacacctgtaattccagcacttcaggaggctgaggcaggaagattgcttgagcccagaaattcaataccagcctgggcaagataatgagaccccatctctgcaaaaaatgaaaaataaactagccaggtgtggtggcatgcacctatgcccccagccactcaagaggctgaggtgggaggatcacttgagcccaggaggtcgaggctgcagtgagctgtgattgcactacactccagcctgggtgacataatgagaccctgtctcaaaaaaaaaaaaaaaaaaaaaaaaaaaactggagccaggcacagtaggacagtagtaattcatgcctgtagtcctagcttacatttcagtagaattgtttagggatattaagtctgttgcttaaacttgtaaaactttattatatattgaaaaacatgcgttgactaattttatgagtattaattgtcttcttttacagtaactggacctctgtcagaactgcaaattgcatatgttagcagagaaacactgcaggtaaatcaagtgctaattcaaaaataacatttttcattaaactaagg

SEQ ID NO: 7 - DNA, 35 bp, Artificial sequence
  Exemplary reporter oligonucleotides
  misc_feature (2)..(2): n is a hydrophobic nucleotide
  misc_feature (34)..(34): n is a hydrophobic nucleotide
cnccttttttttttttttttttttttttttttanc

SEQ ID NO: 8 - DNA, 35 bp, Artificial sequence; identical to SEQ ID NO: 7
  Exemplary reporter oligonucleotides
  misc_feature (2)..(2): z is a hydrophobic nucleotide
  misc_feature (15)..(15): An internal quencher is inserted between residues 15 and 16
  misc_feature (24)..(24): z is a hydrophobic nucleotide
  misc_feature (34)..(34): n is a, c, g, or t

(19) United States
(12) Patent Application Publication
(10) Pub. No.: US 2022/0205024 A1
(43) Pub. Date: Jun. 30, 2022

(54) MELTING TEMPERATURE METHODS, KITS AND REPORTER OLIGO FOR DETECTING VARIANT NUCLEIC ACIDS

(71) Applicant: PENTABASE APS, Odense C (DK)

(72) Inventors: Kamilla Kolding Bendixen, Odense C (DK); Sarah Kronborg Eriksen, Odense C (DK); Emeli Elisabeth Hansen, Odense C (DK); Ulf Bech Christensen, Odense NV (DK); Rasmus Koefoed Tersen, Odense M (DK); Cesilie Lind Madsen, Odense SV (DK)

(73) Assignee: PENTABASE APS, Odense C (DK)

(21) Appl. No.: 17/610,878

(22) PCT Filed: May 13, 2020
(86) PCT No.: PCT/EP2020/063269

§ 371 Date: Nov. 12, 2021

(30) Foreign Application Priority Data
May 13, 2019 (EP) 19174084.4

(51) Int. Cl. C12Q 1/6827; C12Q 1/6818; C12Q 1/6883

(52) U.S. Cl. CPC C12Q 1/6827; C12Q 2600/156; C12Q 1/6883; C12Q 1/6818

(57) ABSTRACT

The present invention relates to melting analysis based methods for detecting the presence of a variant sequence in a target nucleic acid sequence comprising nucleotides of interest, in particular to detect microsatellite instability. The methods employ probes as reporter oligonucleotides with fluorophore and quencher and wherein the nucleotide sequence comprises nucleotides with hydrophobic intercalating residues. Also disclosed are methods for determining efficacy of a drug and for predicting the presence of a clinical disorder in an individual, as well as reporter oligonucleotides and kits for performing the methods.

### TECHNICAL FIELD

The present invention relates to methods for detecting the presence of a variant sequence in a target nucleic acid sequence comprising nucleotides of interest, in particular to detect germline and somatic mutations as well as microsatellite instability. Also disclosed are methods for predicting the efficacy of a drug and for detecting the presence of a clinical disorder in an individual, as well as reporter oligonucleotides and kits for performing the methods.

### BACKGROUND

Heritable (germline) or nonheritable (somatic) mutational events, as single nucleotide substitutions, deletions, insertions, translocations and duplications, exert instrumental impact on human biology. Determination of genetic profile reveals information on response to environmental factors like exercise and diet and in disease diagnosis, prognosis, and applicable treatment regimens. Genetic evaluation is typically performed with tools like DNA sequencing or allele-specific PCR. Methods involving variable binding of molecular probes are applicable for germline variations, but when combined with technologies like HRM also low allele frequency somatic mutations.

Repetitive nucleotide sequences, such as direct or inverted repeats, are observed in many organisms. As an example, hundreds of thousands of microsatellite loci are distributed throughout the human genome and thus, occur statistically about once every 100,000 base pairs. A microsatellite locus is a region of genomic DNA that includes short tandem repeats in which the shortest repetitive units are typically from one to five nucleotides in length. Accordingly, a repetitive unit of a particular microsatellite locus is commonly referred to as a mono-, di-, tri-, tetra- or pentanucleotide repeat locus, as applicable. A given microsatellite locus typically includes between about 10 and 40 of these repetitive units in the tandem arrangement. Further, each microsatellite locus of normal genomic DNA for most diploid species, such as genomic DNA from mammalian species, includes two alleles at each locus. The two alleles can be identical to, or differ from, one another in length and may vary from one individual to the next.

Microsatellite instability (MSI), or replication error (RER), is an example of genomic instability that occurs in certain human neoplasms in which tumor cells have diminished abilities to accurately replicate their DNA. MSI is a common marker of an underlying functional inactivation of a human DNA mismatch repair (MMR) gene. The functional loss of an MMR gene is thought to occur due to biallelic inactivation via coding region mutations, loss of heterozygosity (LOH), and/or promoter methylation. Further, germline mutations of MMR genes are known to be the autosomal dominant genetic defect in most hereditary nonpolyposis colon cancer (HNPCC) kindreds. Other mutations incurred by tumor cells in HNPCC individuals result in biallelic inactivation of the specific MMR gene, causing loss of accurate replication of microsatellite DNA in tumors. MSI is thus a marker of an underlying DNA mismatch repair defect and is also associated with enhanced mutation rates in coding DNA. This mutator phenotype, which results from the MMR defect, causes both coding region base substitutions and frameshift mutations at direct repeats at equal frequencies, in addition to resulting in MSI. The generation of MMR defects and the resultant mutator phenotype are thought to be an early event in tumorigenesis.

Not only is MSI linked to a germline defect in HNPCC families, MSI is also found in about 15 to 20% of sporadic colorectal cancers, where the finding also reflects an overall increase in genomic instability (also measured as tumoral mutation burden). The finding of MSI defects in tumors has also been associated with a better prognosis in stage-for-stage matched tumors. Thus, it is clinically relevant to identify tumors with MSI not only to implicate germline MMR defects (HNPCC families), but also for prognostic stratification. While clinical (Bethesda guidelines (Rodriguez-Bigas et al., 1997) and histopathological features may raise the suspicion that a colorectal tumor is microsatellite-unstable and perhaps has arisen in an HNPCC family, clinicopathological features are often insufficient to diagnose the presence of MSI. Accordingly, molecular testing may be utilized to elucidate the MSI status of a clinically suspicious tumor (Boland et al., 1998).

In addition to colorectal tumors, MSI has also been associated with other types of cancer and other genetic disorders. To illustrate, these include among others, pancreatic carcinomas, gastric carcinomas, bladder cancer, prostate carcinomas, lung cancers, uterine carcinomas and breast cancer. Other exemplary genetic orders thought to be related to microsatellite instability include, e.g., Huntington's disease (HD), dentatorubral and palidoluysian atrophy (DRPLA), spinobulbar and muscular atrophy (SBMA), myotonic dystrophy (DM), fragile X syndrome, FRAXE mental retardation and spinocerebellar ataxias (SCA) Bruton X-linked agammaglobulinemia (XLA), Bloom syndrome (BS), craniofrontonasal syndrome (CFNS) and idiopathic pulmonary fibrosis (IPF).

In view of the above, it is apparent that the analysis of variant nucleotide sequences, for example repetitive nucleotide sequences such as microsatellites, has many diagnostic and prognostic applications among other uses. Sensitive, objective and reliable assays are thus needed.

### SUMMARY

The invention is as defined in the claims. The methods described herein represent a fast, easy, unbiased and sensitive method for investigating germline and somatic mutations as well as microsatellite instability.

Herein is provided a method for detecting the presence of a variant sequence in a target nucleic acid sequence consisting of two strands and comprising nucleotide(s) of interest (NOI) preferably comprising repeats, wherein said target nucleic acid sequence consists of a variant sequence or of a reference sequence, said method comprising the steps of:
a) Providing a first sample comprising nucleic acids suspected of comprising said variant sequence;
b) Providing a second sample comprising nucleic acids comprising said reference sequence, wherein the second sample is a reference sample;
c) Providing a reporter oligonucleotide;
d) Providing a set of primers consisting of a first primer and a second primer, wherein the set of primers together are capable of amplifying the target nucleic acid sequence comprising the NOI;
e) Amplifying the target nucleic acid sequence in the presence of said first sample, said first primer and said second primer, thereby obtaining a first amplicon comprising nucleic acids suspected of comprising a variant sequence; and amplifying the target nucleic acid sequence in the presence of said second sample, said first primer and said second primer, thereby obtaining a second amplicon comprising the reference sequence, wherein the second amplicon is a reference amplicon;
f) Performing melting analysis, such as high-resolution melt (HRM) analysis, of the first amplicon, thereby obtaining a first profile characterised by a first melt curve, and performing melting analysis, such as HRM analysis, of the second amplicon, thereby obtaining a second profile, characterised by a second melt curve wherein the second profile is a reference profile characterised by a reference melt curve; wherein each amplicon comprises a first strand and a second strand, wherein the melting analysis involves hybridisation of the reporter oligonucleotide to one strand of each amplicon, detection of a signal emitted by the fluorophore, and obtaining the first and the second melt curves;
wherein the reporter oligonucleotide is a sequence of in the range of 10 to 50, preferably in the range of 15 to 50 nucleotides, into which in the range of 2 to 10 hydrophobic nucleotides have been inserted,
wherein the reporter oligonucleotide comprises a first fluorophore, preferably in its 5′-end or within 4 nucleotides from the 5′-end, and a first quencher, preferably in its 3′-end or within 4 nucleotides from the 3′-end, and wherein the reporter oligonucleotide comprises a hybridization sequence H, wherein the hybridisation sequence is identical to a consecutive stretch of the sequence of a first strand of the target nucleic acid sequence, and wherein the hybridisation sequence is complementary to a consecutive stretch of the sequence of the second strand of the target nucleic acid sequence, and wherein the hybridisation sequence of the reporter oligonucleotide comprises or consists of a repetitive sequence and at least one helper sequence in its 5′-end and/or in its 3′-end, wherein said helper sequence does not comprise repeats, and can hybridise to the first and second amplicons when the hybridisation sequence is hybridized thereto; and
g) Comparing the first profile to the reference profile, wherein a difference between the first profile and the reference profile indicates that the first sample contains a variant sequence.

Herein is provided a method for detecting the presence of a variant sequence in a target nucleic acid sequence consisting of two strands and comprising nucleotide(s) of interest (NOI), wherein said target nucleic acid sequence consists of a variant sequence or of a reference sequence, said method comprising the steps of:
a) Providing a first sample comprising nucleic acids suspected of comprising said variant sequence;
b) Providing a second sample comprising nucleic acids comprising said reference sequence, wherein the second sample is a reference sample;
c) Providing a reporter oligonucleotide;
d) Providing a set of primers consisting of a first primer and a second primer, wherein the set of primers together are capable of amplifying the target nucleic acid sequence;
e) Amplifying the target nucleic acid sequence in the presence of said first sample, said first primer and said second primer, thereby obtaining a first amplicon comprising nucleic acids suspected of comprising a variant sequence; and amplifying the target nucleic acid sequence in the presence of said second sample, said first primer and said second primer, thereby obtaining a second amplicon comprising the reference sequence, wherein the second amplicon is a reference amplicon;
f) Performing high-resolution melt (HRM) analysis of the first amplicon, thereby obtaining a first HRM profile characterised by a first melt curve, and performing HRM analysis of the second amplicon, thereby obtaining a second HRM profile, characterised by a second melt curve wherein the second HRM profile is a reference profile characterised by a reference melt curve; wherein each amplicon comprises a first strand and a second strand, wherein the HRM analysis involves hybridisation of the reporter oligonucleotide to one strand of each amplicon, detection of a signal emitted by the fluorophore, and obtaining the first and the second melt curves;
wherein the reporter oligonucleotide is a sequence of in the range of 10 to 50 nucleotides into which in the range of 2 to 10 hydrophobic nucleotides have been inserted,
wherein the reporter oligonucleotide comprises a first fluorophore, preferably in its 5′-end or within 4 nucleotides from the 5′-end, and a first quencher, preferably in its 3′-end or within 4 nucleotides from the 3′-end and
wherein the reporter oligonucleotide comprises a hybridization sequence H, wherein
at least one hydrophobic nucleotide is positioned at the 5′-end or within 10 nucleotides from the 5′-end of the reporter oligonucleotide; and/or
at least one hydrophobic nucleotide is positioned at the 3′-end or within 10 nucleotides from the 3′-end of the reporter oligonucleotide; and
wherein the hydrophobic nucleotide has the structure

X—Y-Q

wherein
X is a nucleotide or nucleotide analogue or a backbone monomer unit capable of being incorporated into the backbone of a nucleic acid or nucleic acid analogue,
Q is an intercalator which is not taking part in Watson-Crick hydrogen bonding; and
Y is a linker moiety linking said nucleotide or nucleotide analogue or backbone monomer unit and said intercalator; and wherein the hybridisation sequence is identical to a consecutive stretch of the sequence of a first strand of the target nucleic acid sequence, and wherein the hybridisation sequence is complementary to a consecutive stretch of the sequence of the second strand of the target nucleic acid sequence; and
g) Comparing the first HRM profile to the reference HRM profile, wherein a difference between the first HRM profile and the reference HRM profile indicates that the first sample contains a variant sequence.

Herein is also provided a kit of parts for detecting the presence of a variant sequence in a target nucleic acid sequence consisting of two strands and comprising nucleotide(s) of interest (NOI) preferably comprising repeats, wherein said target nucleic acid sequence consists of a variant sequence or of a reference sequence, said kit of parts comprising:
a) a reporter oligonucleotide comprising a first fluorophore, preferably in its 5′-end or within 4 nucleotides from the 5′-end, and a first quencher, preferably in its 3′-end or within 4 nucleotides from the 3′-end,
   wherein the reporter oligonucleotide is a sequence of in the range of 10 to 50 nucleotides, preferably in the range of 15 to 50 nucleotides, into which in the range of 2 to 10 hydrophobic nucleotides have been inserted and
   wherein the reporter oligonucleotide comprises a hybridization sequence H, and
   wherein the hybridisation sequence is identical to a consecutive stretch of the sequence of a first strand of the target nucleic acid, and wherein the hybridisation sequence is complementary to a consecutive stretch of the sequence of a second strand of the target nucleic acid;
   and wherein the hybridisation sequence of the reporter oligonucleotide comprises or consists of a repetitive sequence and at least one helper sequence in its 5′-end and/or in its 3′-end, wherein said helper sequence does not comprise repeats, and can hybridise to the first and second amplicons when the hybridisation sequence is hybridized thereto; and
b) a set of primers consisting of a first primer and a second primer, wherein the set of primers together are capable of amplifying the target nucleic acid sequence.

Herein is also provided a kit of parts for detecting the presence of a variant sequence in a target nucleic acid sequence consisting of two strands and comprising nucleotide(s) of interest (NOI), wherein said target nucleic acid sequence consists of a variant sequence or of a reference sequence, said kit of parts comprising:
a) a reporter oligonucleotide comprising a first fluorophore, preferably in its 5′-end or within 4 nucleotides from the 5′-end, and a first quencher, preferably in its 3′-end or within 4 nucleotides from the 3′-end,
   wherein the reporter oligonucleotide is a sequence of in the range of 10 to 50 nucleotides into which in the range of 2 to 10 hydrophobic nucleotides have been inserted and wherein the reporter oligonucleotide comprises a hybridization sequence H,
wherein
at least one hydrophobic nucleotide is positioned at the 5′-end or within 10 nucleotides from the 5′-end of the reporter oligonucleotide; and/or
at least one hydrophobic nucleotide is positioned at the 3′-end or within 10 nucleotides from the 3′-end of the reporter oligonucleotide; and
wherein the hydrophobic nucleotide has the structure

X—Y-Q

wherein
X is a nucleotide or nucleotide analogue or a backbone monomer unit capable of being incorporated into the backbone of a nucleic acid or nucleic acid analogue,
Q is an intercalator which is not taking part in Watson-Crick hydrogen bonding; and
Y is a linker moiety linking said nucleotide or nucleotide analogue or backbone monomer unit and said intercalator; and
wherein the hybridisation sequence is identical to a consecutive stretch of the sequence of the first strand of the target nucleic acid comprising the reference sequence, and wherein the hybridisation sequence is complementary to a consecutive stretch of the sequence of the second strand of the target nucleic acid; and
b) a set of primers consisting of a first primer and a second primer, wherein the set of primers together are capable of amplifying the target nucleic acid.

Herein is also provided a reporter oligonucleotide which can hybridise to one strand of a target nucleic acid consisting of two strands and comprising nucleotide(s) of interest (NOI) preferably comprising repeats, said reporter oligonucleotide comprising a first fluorophore, preferably in its 5′-end or within 4 nucleotides from the 5′-end, and a first quencher, preferably in its 3′-end or within 4 nucleotides from the 3′-end, wherein the reporter oligonucleotide is a sequence of in the range of 10 to 50, preferably in the range of 15 to 50 nucleotides, into which in the range of 2 to 10 hydrophobic nucleotides have been inserted and wherein the reporter oligonucleotide comprises a hybridization sequence H,
wherein the hybridisation sequence is identical to a consecutive stretch of the sequence of a first strand of the target nucleic acid, and wherein the hybridisation sequence is complementary to a consecutive stretch of the sequence of a second strand of the target nucleic acid,
and wherein the hybridisation sequence of the reporter oligonucleotide comprises or consists of a repetitive sequence and at least one helper sequence in its 5′-end and/or in its 3′-end, wherein said helper sequence does not comprise repeats, and can hybridise to the second strand of the first and second amplicons when the hybridisation sequence is hybridized thereto.

Herein is also provided a reporter oligonucleotide comprising a first fluorophore, preferably in its 5′-end or within 4 nucleotides from the 5′-end, and a first quencher, preferably in its 3′-end or within 4 nucleotides from the 3′-end, wherein the reporter oligonucleotide is a sequence of in the range of 10 to 50 nucleotides into which in the range of 2 to 10 hydrophobic nucleotides have been inserted and wherein the reporter oligonucleotide comprises a hybridization sequence H,
wherein
at least one hydrophobic nucleotide is positioned at the 5′-end or within 10 nucleotides from the 5′-end of the reporter oligonucleotide; and/or
at least one hydrophobic nucleotide is positioned at the 3′-end or within 10 nucleotides from the 3′-end of the reporter oligonucleotide; and
wherein the hydrophobic nucleotide has the structure

X—Y-Q
wherein
   X is a nucleotide or nucleotide analogue or a backbone monomer unit capable of being incorporated into the backbone of a nucleic acid or nucleic acid analogue,
   Q is an intercalator which is not taking part in Watson-Crick hydrogen bonding; and
   Y is a linker moiety linking said nucleotide or nucleotide analogue or backbone monomer unit and said intercalator; and
      wherein the hybridisation sequence is identical to a consecutive stretch of the sequence of the first strand of the target nucleic acid, and wherein the hybridisation sequence is complementary to a consecutive stretch of the sequence of the second strand of the target nucleic acid.

Herein are also provided methods for predicting the efficacy of treatment of a clinical condition in an individual may comprise the steps of:
a. providing a sample from said individual
b. performing the method for detecting the presence of a variant sequence as described herein to determine whether the sample comprises the variant sequence
wherein the presence of said variant sequence is indicative of whether said drug is efficient in treating said clinical condition in said individual.

Herein are also provided methods useful for predicting or even diagnosing the presence of any clinical condition associated with a particular mutation in an individual, comprising the steps of:
a) Providing a sample from said individual
b) detecting the presence of a mutation associated with the clinical condition in the sample by performing the methods for detecting a variant sequence as described herein;
   wherein the presence of said variant sequence is indicative of said individual suffering from said clinical condition.

### DESCRIPTION OF THE DRAWINGS

FIG. 1 Principle of the method for detecting a variant nucleic acid sequence comprising NOI. (A) A target nucleic acid (black strands) is provided. The second strand comprises NOI (light grey). The first strand is complementary (black line). Arrows indicate primers. The first primer hybridises to the second strand and amplification of the primer takes place (dashed line). The second primer hybridises to the first strand and amplification of the primer takes place (dashed line). For ease of overview only the amplified part of the target nucleic acid is shown. The second primer is here provided in excess compared to the first primer (asymmetric PCR). Hence, more DNA is generated which comprises the NOI sequence (dashed black and dashed grey line). (B) The amplified DNA consists of a mixture of double stranded target nucleic acids and single stranded target nucleic acids comprising NOI. Here is shown the single stranded sequence, comprising the NOI (light grey). The reporter oligonucleotide (RO) hybridises to that strand. It comprises here a fluorophore F′ (circle) and quenchers Q (squares). A melt curve is obtained (x-axis: T is temperature; y-axis: F is fluorescence).

FIG. 2 Microsatellite instability does not necessarily lead to significant changes in melting temperature. BAT25 assay using asymmetric PCR. X axis shows the temperature. (A) Melt curves, where bilinear normalization and temperature shift with an intensity threshold of 0.1 RFU were applied. The difference between the curves, measured as the maximal difference in amplitude between the curves, was ˜0.07 RFU. (B) First negative derivative curves, without applying temperature shift, the normal tissue has a Tₘ of 57.31° C. and the tumour tissue a Tₘ of 57.41° C.

FIG. 3 Bilinear normalization makes the slope of the melt curves closer to 0 prior to and after the actual melt phase. The microsatellite NR22 is analysed using asymmetric PCR and a reporter oligonucleotide. X axis shows the temperature. HRM curves were obtained with: (A) standard normalisation, no temperature shift; (B) bilinear normalisation, no temperature shift.

FIG. 4 Bilinear normalization makes the slope of the melt curves closer to 0 prior to and after the actual melt. Difference HRM curves from the melt curves of FIG. 3, where the reference HRM curve is set as baseline, which is subtracted from the other (here the first) melt curve. X axis shows the temperature. (A) no bilinear normalisation, no temperature shift; (B) bilinear normalisation, no temperature shift.

FIG. 5 In theory, there should not be any differences in melting temperatures between a normal and a tumour tissue sample from a microsatellite stable person. However, differences in amounts, salt concentrations, impurities and other variables between samples can lead to small differences in melting temperatures. However, bilinear normalization and temperature shift can reduce the difference between normal and tumour tissue of a microsatellite stable patient. NR24 assay using asymmetric PCR and a reporter oligonucleotide. X axis shows the temperature. (A) HRM profiles, where standard normalization was applied. The difference between the curves, measured as the maximal difference in fluorescence at a given temperature between the normalization areas on difference graphs, was ˜0.04 RFU. X axis shows the temperature. (B) HRM profiles, where bilinear normalization was applied but temperature shift threshold was not applied. The difference between the curves was ˜0.02 RFU. (C) HRM curves, where bilinear normalization and temperature shift with an intensity threshold of 0.1 RFU were applied. The difference between the curves was ˜0.01 RFU.

FIG. 6 Temperature shift can neutralize the change in melting temperature caused by different salt concentration in DNA buffers. NR24 assay using asymmetric PCR and a reporter oligonucleotide. X axis shows the temperature. (A) HRM curves, where bilinear normalization was applied, but temperature shift was not applied. The difference between the curves, measured as the maximal difference in fluorescence at a given temperature between the normalization areas on difference graphs, was ˜0.07 RFU. (B) HRM curves, where bilinear normalization and temperature shift intensity threshold of 0.1 RFU were applied. The difference between the curves was ˜0.01 RFU.

FIG. 7 Assay for investigation of the microsatellite MONO27 using asymmetric PCR for 16 normal tissue DNA samples. HRM curves were obtained with: (A) bilinear normalisation, no temperature shift; (B) bilinear normalisation, and temperature shift with an intensity threshold of 0.1 RFU. X axis shows the temperature. Applying a temperature shift allows the use of one sample as universal reference.

FIG. 8 Difference plots of the HRM curves of FIG. 7. (A) Bilinear normalisation, no temperature shift; (B) bilinear normalisation, and temperature shift with an intensity threshold of 0.1 RFU. X axis shows the temperature.

FIG. 9 Asymmetric PCR creates more single stranded target amplicon. NR22 assays using symmetric and asymmetric PCR. X axis shows the number of PCR cycles and Y axis the fluorescence.

FIG. 10 Asymmetric PCR creates higher signal to noise and sharper melt (i.e. more narrow melt peaks). NR22 assay using symmetric and asymmetric PCR. (A) melt curves; (B): melt curves, normalised using standard normalisation. X axis shows the temperature.

FIG. 11 Asymmetric PCR makes it easier to discriminate between MSS and MSI patients. Shown is data using NR22 assay. HRM curves were obtained after applying standard normalisation with asymmetric PCR (A) or with symmetric PCR (B). X axis shows the temperature.

FIG. 12 Asymmetric PCR makes it easier to discriminate between MSS and MSI patients. Shown is data using NR22 assay. Difference plots from the HRM curves of FIG. 11. (A) asymmetric PCR; (B): symmetric PCR. X axis shows the temperature.

FIG. 13 Single-end overhang in the reporter oligonucleotide can increase melting temperature. Shown is data based on NR22 assay using asymmetric PCR using a reporter oligonucleotide with two different reporter oligonucleotides. (A) HRM curves. (B) Negative first derivative of the HRM curves. X axis shows the temperature.

FIG. 14 Double-quenching the reporter oligonucleotide increases the signal to noise ratio. Shown is data using BAT26 assay using asymmetric PCR. HRM curves (A) and negative first derivative curves (B) using a single-quenched (“Not DQ probe”) and a double-quenched (“DQ probe”) reporter oligonucleotides. X axis shows the temperature.

FIG. 15 Additional nucleotide repeats in the hybridisation sequence of the reporter oligonucleotide makes it possible to detect longer microsatellites. Shown is data using NR21 assay using asymmetric PCR. X axis shows the temperature.

FIG. 16 A single point mutation changes meting temperature with several degrees. KIT Exon 13 assay was used for the experiment. The PCR was carried out as asymmetric PCR. X axis shows the temperature. Dashed lines show the results against a wild type target nucleic acid and solid line shows results against a target nucleic acid comprising a mutation. (A) Normalized HRM curves; Y axis show fluorescence. (B) Melt peaks (negative first derivatives of melt curves). Y axis is −dF/dT.

FIG. 17 A strong helper sequence helps discriminate between wild type and mutant. NR24 assay was used for the experiment. The PCR was carried out as asymmetric PCR. X axis shows the temperature, and y axis shows fluorescence. Dashed lines show the results against a wild type target nucleic acid and solid line shows results against a target nucleic acid comprising a mutation. (A) Normalised HRM curves with a first reporter oligonucleotide comprising a first helper sequence. (B) Normalised HRM curves with a second reporter oligonucleotide comprising a second helper sequence, stronger than the first helper sequence.

FIG. 18 A strong helper sequence helps discriminate between wild type and mutant. NR24 assay was used for the experiment. The PCR was carried out as asymmetric PCR. X axis shows the temperature, and y axis shows fluorescence. Dashed lines show the results against a wild type target nucleic acid and solid line shows results against a target nucleic acid comprising a mutation. (A) Difference plots with a first reporter oligonucleotide comprising a first helper sequence. (B) Difference plots with a second reporter oligonucleotide comprising a second helper sequence, stronger than the first helper sequence.

### DETAILED DESCRIPTION

### Definitions

Amplicon

An “amplicon” refers to a molecule made by copying or transcribing another molecule. Exemplary processes in which amplicons can be produced include transcription, cloning, and/or a polymerase chain reaction (PCR) or another nucleic acid amplification technique (e.g., strand displacement PCR amplification (SDA), duplex PCR amplification, etc.). Typically, an amplicon is a copy of a selected nucleic acid (e.g., a template or target nucleic acid) or is complementary thereto.

Bilinear Normalization

The term “Bilinear normalization” is used herein to refer to a mathematical transformation applied to the curve(s) by scaling of the fluorescence data of different curves to normalized curves. This allows for the comparison of different curves removing other factors that can influence the fluorescence signal (such as different signal strengths among different positions in the instrument, different transparency of plastic and other factors introducing variables of fluorescence measurements). Bilinear normalization forces the curve(s) to have the same value (at the mean value of the normalization area) and the curve course as horizontally (flat) as possible in the selected normalization areas. Several algorithms are known in the art, which can be applied to a curve, transforming the curve(s) into curve(s) that allow for comparison of different melt curves. The normalization also reduces the influence of different optics and mechanical differences between wells.

Hydrophobic Nucleotide

The term “hydrophobic nucleotide” as used herein refers to the hydrophobic nucleotides described in detail herein below in the section “hydrophobic nucleotide”. In particular, a hydrophobic nucleotide according to the invention contains an intercalator connected to a nucleotide/nucleotide analogue/backbone monomer unit via a linker.

Melting Temperature

The term “melting temperature” as used herein denotes the temperature in degrees centigrade at which 50% helical (hybridised) versus coil (unhybridised) forms are present. Melting temperature may also be referred to as (Tₘ). Melting of nucleic acids and nucleic acid analogues refers to thermal separation of the two strands of a double-stranded nucleic acid molecule.

Microsatellite

The terms “microsatellite”, “microsatellite marker” or “microsatellite locus” refer to a region of genomic DNA that includes tandem nucleotide repeats. These repeats or “repetitive units” are typically from about one to about seven base pairs in length. Microsatellite loci typically include between about 10 to 40 of these repetitive units in a tandem arrangement. In addition to mononucleotide repeats, which are repeats of one nucleotide, other exemplary repetitive nucleotide sequences include dinucleotide repeats, for example AT repeats and GC repeats, trinucleotide repeats, for example CGG repeats, CGC repeats, TAT repeats, ATT repeats, tetranucleotide repeats, pentanucleotide repeats and/or complementary repeats thereof.

Nucleotide Analogue

The term “nucleotide analogue” comprises all nucleotide analogues capable of being incorporated into a nucleic acid backbone and capable of specific base-pairing, essentially like naturally occurring nucleotides. Nucleotide analogues according to the present invention include, but are not limited to the nucleotide analogues selected from the group consisting of PNA, HNA, MNA, ANA, LNA, XNA, INA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R₁-RNA, 2′-OR₁—RNA (R₁ being a substituent), α-L-RNA, α-D-RNA, and β-D-RNA.

Nucleotide of Interest

The term “nucleotide(s) of interest” as used herein refers to nucleotide(s) within a target nucleic acid sequence, which may be present in two different variants. “Nucleotide(s) of interest” may also be referred to as “NOI” herein. Thus, the NOI may consist of a variant sequence or it may consist of a reference sequence, also referred to as the “reference sequence” herein. In some embodiments, the reference sequence may be a wild type sequence.

Nucleotide

The term “nucleotide” as used herein refers to naturally occurring nucleotides, for example naturally occurring ribonucleotides or deoxyribonucleotides or naturally occurring derivatives of ribonucleotides or deoxyribonucleotides. Naturally occurring nucleotides include deoxyribonucleotides comprising one of the four nucleobases adenine (A), thymine (T), guanine (G) or cytosine (C), and ribonucleotides comprising one of the four nucleobases adenine (A), uracil (U), guanine (G) or cytosine (C).

Oligonucleotide

The term “oligonucleotide” as used herein refers to oligomers of nucleotides and/or nucleotide analogous and/or hydrophobic nucleotides. Preferably, and oligonucleotide is an oligomer of nucleotides optionally comprising one or more hydrophobic nucleotides.

Reference Target Sequence

The term “reference target sequence” refers to a stretch of the target nucleic acid sequence comprising the reference sequence.

Standard Normalization

The term “standard normalization” refers to the scaling of fluorescence data of different curves to normalized curves and allows for the comparison of different curves removing other factors that can influence the fluorescence signal (such as different signal strengths among different positions in the instrument, different transparency of plastic and other factors introducing variables of fluorescence measurements).

Target Nucleic Acid Sequence

The term “target nucleic acid sequence” as used herein refers to a nucleic acid sequence comprising the nucleotide(s) of interest (NOI). The target nucleic acid sequence can be amplified using a set of primers.

Temperature Shift

The term “temperature shift” as used herein refers to a mathematical tool that is applied to two or more normalized curves, shifting these in the temperature direction to have exactly the same Tₘ at a given fluorescence limit (intensity threshold). Applying such a transformation algorithm to a data set reduces the influence from for example different salt concentrations in different samples.

Variant Sequence

The term “variant sequence” as used herein refers to nucleotide(s) within a target nucleic acid sequence, which are different to a reference sequence. Thus, the target nucleic acid sequence may contain a NOI, which is the variant sequence, or the target nucleic acid sequence may contain NOI, which is the reference sequence or the NOI may even be yet another variant sequence. The variant sequence may for example be a mutation, such as a single nucleotide mutation or it may be an insertion or deletion. The variant sequence and the reference sequence may overlap so that the sequence of one comprises the entire sequence of the other. In such cases, the variant sequence and the reference sequence may differ in the number of nucleotides making up the sequence.

Methods for Detecting the Presence of a Variant Sequence in a Target Nucleic Acid Sequence

The invention relates to methods for detection of a variant sequence in a target nucleic acid sequence. Thus, the methods are in particular useful for detecting the presence of a particular sequence in a target nucleic acid sequence, which may occur with two or more different sequences. The methods can for example be used for distinguishing between a wild type and a mutant sequence and for distinguishing between different polymorphic sequences. The methods are particularly useful for detecting variant sequences resulting from microsatellite instability by detecting the presence of variant sequences having a length different from a wild type or reference sequence. The methods can thus advantageously be used for detecting a target nucleic acid sequence comprising repeats, in particular microsatellites.

Herein is provided a method for detecting the presence of a variant sequence in a target nucleic acid sequence consisting of two strands and comprising nucleotide(s) of interest (NOI) preferably comprising repeats, wherein said target nucleic acid sequence consists of a variant sequence or of a reference sequence, said method comprising the steps of:
a) Providing a first sample comprising nucleic acids suspected of comprising said variant sequence;
b) Providing a second sample comprising nucleic acids comprising said reference sequence, wherein the second sample is a reference sample;
c) Providing a reporter oligonucleotide;
d) Providing a set of primers consisting of a first primer and a second primer, wherein the set of primers together are capable of amplifying the target nucleic acid sequence comprising the NOI;
e) Amplifying the target nucleic acid sequence in the presence of said first sample, said first primer and said second primer, thereby obtaining a first amplicon comprising nucleic acids suspected of comprising a variant sequence; and amplifying the target nucleic acid sequence in the presence of said second sample, said first primer and said second primer, thereby obtaining a second amplicon comprising the reference sequence, wherein the second amplicon is a reference amplicon;
f) Performing melting analysis, such as high-resolution melt (HRM) analysis, of the first amplicon, thereby obtaining a first profile characterised by a first melt curve, and performing melting analysis, such as HRM analysis, of the second amplicon, thereby obtaining a second profile, characterised by a second melt curve wherein the second profile is a reference profile characterised by a reference melt curve; wherein each amplicon comprises a first strand and a second strand, wherein the melting analysis involves hybridisation of the reporter oligonucleotide to one strand of each amplicon, detection of a signal emitted by the fluorophore, and obtaining the first and the second melt curves;
wherein the reporter oligonucleotide is a sequence of in the range of 10 to 50, preferably in the range of 15 to 50 nucleotides, into which in the range of 2 to 10 hydrophobic nucleotides have been inserted,
wherein the reporter oligonucleotide comprises a first fluorophore, preferably in its 5′-end or within 4 nucleotides from the 5′-end, and a first quencher, preferably in its 3′-end or within 4 nucleotides from the 3′-end, and
wherein the reporter oligonucleotide comprises a hybridization sequence H, wherein the hybridisation sequence is identical to a consecutive stretch of the sequence of a first strand of the target nucleic acid sequence, and wherein the hybridisation sequence is complementary to a consecutive stretch of the sequence of the second strand of the target nucleic acid sequence,
and wherein the hybridisation sequence of the reporter oligonucleotide comprises or consists of a repetitive sequence and at least one helper sequence in its 5′-end and/or in its 3′-end, wherein said helper sequence does not comprise repeats, and can hybridise to the first and second amplicons when the hybridisation sequence is hybridized thereto; and
g) Comparing the first profile to the reference profile, wherein a difference between the first profile and the reference profile indicates that the first sample contains a variant sequence.

Herein is thus provided a method for detecting the presence of a variant sequence in a target nucleic acid sequence consisting of two strands and comprising nucleotide(s) of interest (NOI), wherein said target nucleic acid sequence consists of a variant sequence or of a reference sequence, said method comprising the steps of:
a) Providing a first sample comprising nucleic acids suspected of comprising said variant sequence;
b) Providing a second sample comprising nucleic acids comprising said reference sequence, wherein the second sample is a reference sample;
c) Providing a reporter oligonucleotide;
d) Providing a set of primers consisting of a first primer and a second primer, wherein the set of primers together are capable of amplifying the target nucleic acid sequence;
e) Amplifying the target nucleic acid sequence in the presence of said first sample, said first primer and said second primer, thereby obtaining a first amplicon comprising nucleic acids suspected of comprising a variant sequence; and amplifying the target nucleic acid sequence in the presence of said second sample, said first primer and said second primer, thereby obtaining a second amplicon comprising the reference sequence, wherein the second amplicon is a reference amplicon;
f) Performing high-resolution melt (HRM) analysis of the first amplicon, thereby obtaining a first HRM profile characterised by a first melt curve, and performing HRM analysis of the second amplicon, thereby obtaining a second HRM profile, characterised by a second melt curve wherein the second HRM profile is a reference profile characterised by a reference melt curve; wherein each amplicon comprises a first strand and a second strand, wherein the HRM analysis involves hybridisation of the reporter oligonucleotide to one strand of each amplicon, detection of a signal emitted by the fluorophore, and obtaining the first and the second melt curves;
wherein the reporter oligonucleotide is a sequence of in the range of 10 to 50 nucleotides into which in the range of 2 to 10 hydrophobic nucleotides have been inserted,
wherein the reporter oligonucleotide comprises a first fluorophore, preferably in its 5′-end or within 4 nucleotides from the 5′-end, and a first quencher, preferably in its 3′-end or within 4 nucleotides from the 3′-end and
wherein the reporter oligonucleotide comprises a hybridization sequence H, wherein
at least one hydrophobic nucleotide is positioned at the 5′-end or within 10 nucleotides from the 5′-end of the reporter oligonucleotide; and/or
at least one hydrophobic nucleotide is positioned at the 3′-end or within 10 nucleotides from the 3′-end of the reporter oligonucleotide; and
wherein the hydrophobic nucleotide has the structure

X—Y-Q
wherein
   X is a nucleotide or nucleotide analogue or a backbone monomer unit capable of being incorporated into the backbone of a nucleic acid or nucleic acid analogue,
   Q is an intercalator which is not taking part in Watson-Crick hydrogen bonding; and
   Y is a linker moiety linking said nucleotide or nucleotide analogue or backbone monomer unit and said intercalator; and
   wherein the hybridisation sequence is identical to a consecutive stretch of the sequence of a first strand of the target nucleic acid sequence, and wherein the hybridisation sequence is complementary to a consecutive stretch of the sequence of the second strand of the target nucleic acid sequence; and
g) Comparing the first HRM profile to the reference HRM profile, wherein a difference between the first HRM profile and the reference HRM profile indicates that the first sample contains a variant sequence.

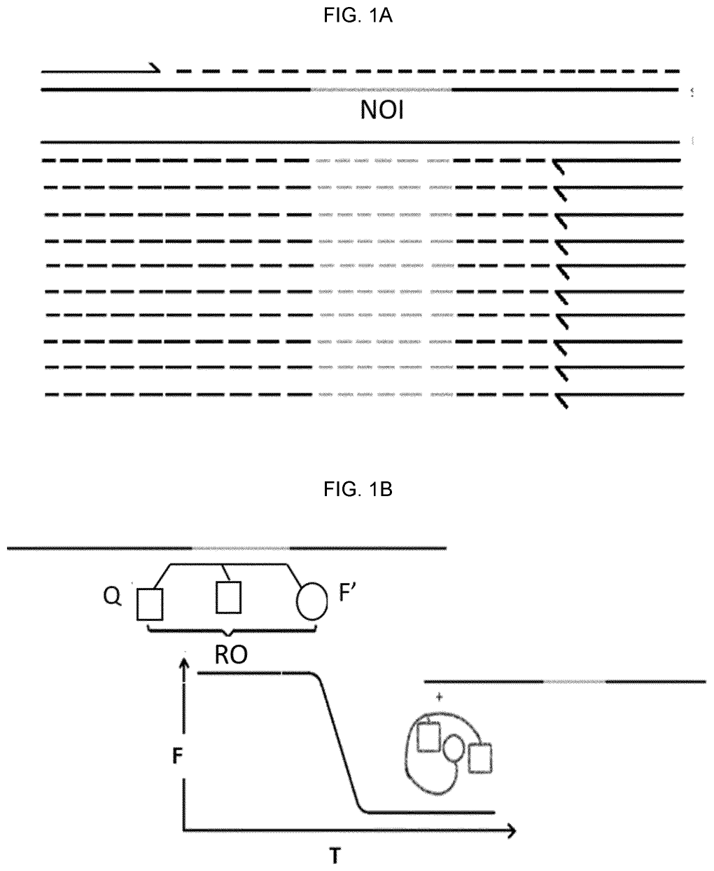

Accordingly, when two or more different target nucleic acid sequences exist, the methods are useful for distinguishing between at least two different target nucleic acid sequences, i.e. the target nucleic acid sequence comprising a variant sequence, and the target nucleic acid sequence not comprising the variant sequence. The latter may also be referred to as “reference sequence”. Optionally, the methods may be useful for distinguishing also between target nucleic acid sequences comprising other variant sequences. The general principle of the method is exemplified in FIG. 1. In short, a difference between the first and second profiles, in particular a difference between the analysed melt profile and the reference melt profile, indicates that the tested sample (the first sample) contains a variant sequence; it will be understood that throughout this disclosure a difference preferably refers to a significant difference.

In embodiments of the invention where the methods are used for distinguishing between a wild type and a mutant sequence, the mutant sequence may be the reference sequence. However, frequently, the wild type sequence will be the reference sequence. The methods of the invention may also be used for distinguishing between a wild type and several different mutant sequences, in which case the wild type sequence typically will be the reference sequence and the several different mutant sequences will be variant sequences. In some embodiments of the methods, the reference sequence is a microsatellite sequence. The variant sequence may then be a microsatellite sequence, consisting of a number of tandem repeats, having a number of tandem repeats different than the number of tandem repeats of the reference sequence. For example, the variant sequence may be the result of microsatellite instability—in these cases, the variant sequence may be a plurality of variant sequences having different numbers of tandem repeats. By comparison, the reference sequence would then have a constant number of tandem repeats.

The variant sequence may in some embodiments be a mutation indicative of a disease state or it may be predictive for the efficacy of a given treatment.

Nucleotide(s) of Interest (NOI), Variant Sequence and Reference Sequence

The variant sequence may be any sequence, which differs from another sequence, in particular a reference sequence. Frequently, the variant sequence is a mutant sequence, e.g. a sequence which differs from a wild type sequence, for example because of the presence of mutations replacing a nucleotide by another, and/or because of the insertion and/or deletion of nucleotides compared to the reference sequence. The variant sequence can however also be a polymorphic sequence or any other sequence which differs from a reference sequence. Preferably, the variant sequence is a variant of a microsatellite, having a different length than the normal length of the microsatellite. The present methods are indeed particularly useful for detecting microsatellite instability. They can be used to detect instability of short (less than 15 nucleotides) microsatellites, as well as longer microsatellites (more than 15 nucleotides). In a subject with microsatellite instability, longer microsatellites typically will mutate before shorter microsatellites—hence it may be advantageous to use a target nucleic acid which is a longer microsatellite, having a length of 15 nucleotides or more.

The variant sequence may consist of at least one, such as 1, for example 2, such as 3, for example 4, such as 5, for example 6, such as 7, for example 8, such as 9, for example 10, such as from 10 to 20, for example from 20 to 50, such as more than 50 nucleotides. Preferably, the variant sequence consists of 10 nucleotides or more, such as 15 nucleotides or more. Thus in some embodiments, the variant sequence consist of 11, 12, 13, 14, 15, 16, 17, 19, 20 nucleotides or more, such as 25, 30, 35, 40, 45 or 50 nucleotides or more. For example, the variant sequence consists of 20, 21, 22, 23, 24, 25, 26, 27, 28, 29 or 30 nucleotides or more.

In some embodiments of the invention, the variant sequence is a single nucleotide mutation or a single nucleotide polymorphism (SNP).

The variant sequence may be a change of one or more nucleotides for one or more other nucleotides compared to the reference target sequence. Furthermore, the term variant sequence may be a deletion or insertion of nucleotides within a nucleic acid, for example deletion or insertion of nucleotides compared to the reference target sequence.

The target nucleic acid sequence may comprise a polymorphic site (see details herein below) and thus the reference target sequence may comprise one polymorphism, whereas the “variant sequence” may constitute another polymorphism.

In one embodiment, the reference target sequence is a wild type sequence, i.e. the most frequently naturally occurring sequence, whereas the variant sequence comprises one or more mutations, insertions or deletions compared to said wild type sequence. Accordingly, a variant sequence according to the present invention may in one embodiment be a polymorphism, such as a single nucleotide polymorphism (SNP). For example, the polymorphism may be indicative of a specific DNA profile. Knowledge of a specific DNA profile may for example be employed to identify an individual. For example, a specific DNA profile may be employed to identify a criminal or a potentially criminal or to identify a dead body or part of a dead body. Furthermore, a specific DNA profile may be employed to determine relationship between individuals, for example parents-child relationship or more distant relationships. Relationship may also be relationship between different species or different population of a given species.

In some embodiments, the reference sequence is or comprises a microsatellite having a given number of repeats. The variant sequence may then be one or several variant sequences, having a number of repeats different from the number observed in the reference or wild type sequence.

In one embodiment, the variant sequence may be indicative of a clinical condition or the mutation may be indicative of increased risk of a clinical condition. In particular, the variant sequence may be a microsatellite and the clinical condition may be associated with microsatellite instability.

Said clinical condition may for example be selected from the group consisting of neoplastic diseases, neurodegenerative diseases, cardiovascular diseases and metabolic disorders including diabetes.

Furthermore, the variant sequence may be indicative of a specific response to a predetermined drug treatment. For example, the presence of the variant sequence may be indicative of whether an individual will respond positively to said drug treatment or whether an individual can or cannot tolerate a specific drug treatment. For example, the detection of MSI could be used in the decision process of prescribing the use of check point inhibitors like e.g. Keytruda® (pembrolizumab, Merck & Co. Inc.) or Opdivo (nivolumab, Bristol-Myers Squibb).

The variant sequence may be positioned in a particular gene, a gene segment, a microsatellite or any other DNA sequence. Furthermore, the variant sequence may be positioned in a mRNA, miRNA or any other RNA sequence. The methods described herein enable the detection of particular DNAs, which may be of eukaryotic, prokaryotic, Archae or viral origin. For example, the invention may assist in the diagnosis and/or genotyping of various infectious diseases by assaying for particular sequences known to be associated with a particular microorganism.

Target Nucleic Acid Sequence

The present methods are for detecting the presence of a variant sequence in a target nucleic acid sequence consisting of two strands and comprising nucleotide(s) of interest (NOI). While the target nucleic acid sequence consists of two strands and is typically DNA, it will be understood that the present methods can easily be adapted to detecting the presence variant sequence in a target nucleic acid by amplifying a single strand of the target nucleic acid sequence—for example if the amplification is amplification of RNA. Thus the present methods can also be used for detecting a variant sequence in a target RNA, which results for example from the transcription of the target nucleic acid sequence consisting of two strands.

The target nucleic acid is thus the sequence of interest, which is suspected of comprising a variant sequence. In other words, it often corresponds to a specific locus.

The target nucleic acid sequence may consist of at least one, such as 1, for example 2, such as 3, for example 4, such as 5, for example 6, such as 7, for example 8, such as 9, for example 10, such as from 10 to 20, for example from 20 to 50, such as more than 50 nucleotides. Preferably, the target nucleic acid sequence consists of 15 nucleotides or more. Thus in some embodiments, the target nucleic acid sequence consists of 11, 12, 13, 14, 15, 16, 17, 19, 20 nucleotides or more, such as 25, 30, 35, 40, 45 or 50 nucleotides or more. For example, the target nucleic acid sequence consists of 20, 21, 22, 23, 24, 25, 26, 27, 28, 29 or 30 nucleotides or more. Because the present methods are particularly useful for detecting variant sequences of microsatellites, particularly of microsatellites having a length of 15 nucleotides or more, where the target nucleic acid sequence is the reference sequence, its length is preferably 15 nucleotides or more.

In some embodiments, the target nucleic acid is a microsatellite, comprising a number of tandem repeats. For the reference sequence, the number of tandem repeats is M, where M is an integer. For the reference sequence, the total length is n nucleotides. The variant sequence has M′ tandem repeats and a total length of n′ nucleotides. M and M′ are different integers. n and n′ are different integers. It is however possible that some of the variant sequences have M tandem repeats and a total length of n nucleotides—however, in this case, some or most of the variant sequences will still have a different number of repeats and a different total length compared to the reference sequence, which will enable the present methods to detect the presence of variant sequences which differ from the reference sequence.

In some embodiments, n and/or n′ is 1, such as 1, for example 2, such as 3, for example 4, such as 5, for example 6, such as 7, for example 8, such as 9, for example 10, such as from 10 to 20, for example from 20 to 50, such as more than 50 nucleotides. Preferably, n and/or n′ is 15 nucleotides or more; in some embodiments, at least n is 15 nucleotides or more. Thus in some embodiments, n and/or n′ consists of 11, 12, 13, 14, 15, 16, 17, 18, 19, 20 nucleotides or more, such as 25, 30, 35, 40, 45 or 50 nucleotides or more. For example, n and/or n′ consists of 20, 21, 22, 23, 24, 25, 26, 27, 28, 29 or 30 nucleotides or more. Preferably, at least n consists of 15, 16, 17, 18, 19, 20 nucleotides or more, such as 20, 21, 22, 23, 24, 25, 26, 27, 28, 29 or 30 nucleotides or more, such as 25, 30, 35, 40, 45 or 50 nucleotides or more. In some embodiments, n′ is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotides or more, such as 25, 30, 35, 40, 45 or 50 nucleotides or more.

The present methods can be used to detect a variant sequence which may be indicative of a specific disorder. For example, the disorder is associated with microsatellite instability.

It is often advantageous to investigate a sample for the presence of several different variant sequences. Accordingly, the present methods may be adapted, so that several variant sequences are detected simultaneously. Sometimes it is sufficient just to determine if at least one of many variant sequences is present.

In some embodiments, the target nucleic acid sequence is a plurality of target nucleic acid sequences comprising one or more of BAT25, BAT26, NR21, NR22, NR24 and

MONO27, which are microsatellite markers comprising mononucleotide repeats. The methods allow detection of variant sequences having a different number of repeats than the reference sequence for these target nucleic acid sequences, and hence a different total length.

The reference or wild type sequence of human BAT25 is as set forth in SEQ ID NO: 1. The mononucleotide repeats of BAT25 correspond to position 147 to 171 of SEQ ID NO: 1. The reference sequence thus has M mononucleotide repeats, where M=25.

The reference or wild type sequence of human BAT26 is as set forth in SEQ ID NO: 2. The mononucleotide repeats of BAT26 correspond to position 222 to 248 of SEQ ID NO: 2. The reference sequence thus has M mononucleotide repeats, where M=27.

The reference or wild type sequence of human NR21 is as set forth in SEQ ID NO: 3. The mononucleotide repeats of NR21 correspond to position 189 to 209 of SEQ ID NO: 3. The reference sequence thus has M mononucleotide repeats, where M=21.

The reference or wild type sequence of human NR22 is as set forth in SEQ ID NO: 4. The mononucleotide repeats of NR22 correspond to position 152 to 172 of SEQ ID NO: 4. The reference sequence thus has M mononucleotide repeats, where M=21.

The reference or wild type sequence of human NR24 is as set forth in SEQ ID NO: 5. The mononucleotide repeats of NR24 correspond to position 165 to 187 of SEQ ID NO: 5. The reference sequence thus has M mononucleotide repeats, where M=23.

The reference or wild type sequence of human MONO27 is as set forth in SEQ ID NO: 6. The mononucleotide repeats of MONO27 correspond to position 300 to 327 of SEQ ID NO: 6. The reference sequence thus has M mononucleotide repeats, where M=28.

In some embodiments, the target nucleic acid sequence is BAT25, as set forth in SEQ ID NO: 1, and the NOI correspond to the sequence defined by positions 147 to 171 of SEQ ID NO: 1. In other embodiments, the target nucleic acid sequence is BAT26, as set forth in SEQ ID NO: 2, and the NOI correspond to the sequence defined by positions 222 to 248 of SEQ ID NO: 2. In other embodiments, the target nucleic acid sequence is NR21, as set forth in SEQ ID NO: 3, and the NOI correspond to the sequence defined by positions 189 to 209 of SEQ ID NO: 3. In other embodiments, the target nucleic acid sequence is NR22, as set forth in SEQ ID NO: 4, and the NOI correspond to the sequence defined by positions 152 to 172 of SEQ ID NO: 4. In other embodiments, the target nucleic acid sequence is NR24, as set forth in SEQ ID NO: 5, and the NOI correspond to the sequence defined by positions 165 to 187 of SEQ ID NO: 5. In other embodiments, the target nucleic acid sequence is MONO27, as set forth in SEQ ID NO: 6, and the NOI correspond to the sequence defined by positions 300 to 327 of SEQ ID NO: 6.

In other embodiments, the target nucleic acid sequence is two target nucleic acid sequences. For example, the two target nucleic acid sequences are BAT25 and BAT26; BAT25 and NR21; BAT25 and NR22; BAT25 and NR24; BAT25 and MONO27; BAT26 and NR21; BAT26 and NR22; BAT26 and NR24; BAT26 and MONO27; NR21 and NR22; NR21 and NR24; NR21 and MONO27; NR22 and NR24; NR22 and MONO27; NR24 and MONO27, where the NOIs are as defined above.

In other embodiments, the target nucleic acid sequence is three target nucleic acid sequences. For example, the three target nucleic acid sequences are BAT25, BAT26 and NR21; BAT25, BAT26 and NR22; BAT25, BAT26 and NR24; BAT25, BAT26 and MONO27; BAT25, NR21 and NR22; BAT25, NR21 and NR24; BAT25, NR21 and

MONO27; BAT25, NR22 and NR24; BAT25, NR22 and MONO27; BAT25, NR24 and MONO27; BAT26, NR21 and NR22; BAT26, NR21 and NR24; BAT26, NR21 and MONO27; BAT26, NR22 and NR24; BAT26, NR22 and MONO27; BAT26, NR24 and MONO27; NR21, NR22 and NR24; NR21, NR22 and MONO27; NR21, NR24 and MONO27; NR22, NR24 and MONO27, where the NOIs are as defined above.

In other embodiments, the target nucleic acid sequence is four target nucleic acid sequences. For example, the four target nucleic acid sequences are BAT25, BAT26, NR21 and NR22; BAT25, BAT26, NR21 and NR24; BAT25, BAT26, NR21 and MONO27; BAT25, BAT26, NR22 and NR24; BAT25, BAT26, NR22 and MONO27; BAT26, NR21, NR22 and NR24; BAT26, NR21, NR22 and MONO27; BAT26, NR21, NR24 and MONO27; NR21, NR22, NR24 and MONO27, where the NOIs are as defined above.

In other embodiments, the target nucleic acid sequence is five target nucleic acid sequences. For example, the five target nucleic acid sequences are BAT25, BAT26, NR21, NR22 and NR24; BAT25, BAT26, NR21, NR22 and MONO27; BAT25, BAT26, NR22, NR24 and MONO27; BAT26, NR21, NR22, NR24 and MONO27, where the NOIs are as defined above.

In other embodiments, the target nucleic acid sequence is six target nucleic acid sequences. For example, the six target nucleic acid sequences are BAT25, BAT26, NR21, NR22, NR24 and MONO27, where the NOIs are as defined above.

In preferred embodiments, the target nucleic acid is five target nucleic acids, preferably BAT25, BAT26, NR21, NR22 and NR24; or BAT25, BAT26, NR22, NR24 and MONO27, where the NOIs are as defined above. In other preferred embodiments, the target nucleic acid sequence is six target nucleic acid sequences, preferably BAT25, BAT26, NR21, NR22, NR24 and MONO27, where the NOIs are as defined above.

Samples

In a first step of the method, a first sample is provided, which comprises nucleic acids suspected of comprising a variant sequence to be detected. In a second step of the method, a second sample is provided, which comprises the reference sequence. The second sample is thus a reference sample, and the terms will be used herein interchangeably.

The sample may comprise cells comprising said nucleic acids. The cells may for example be prokaryotic cells or eukaryotic cells, such as plant cells or mammalian cells.

The sample may for example be a synthetically prepared sample, which may or may not have been further processed in vitro, however most frequently, the sample is a sample obtained from an individual.

In some embodiments, the first sample is a sample obtained from an individual suffering from or suspected of suffering from a disease or disorder characterised by the presence of a variant sequence, and the second sample is a sample obtained from a healthy individual. It is also possible to use a first sample comprising cells e.g. from a diseased tissue, i.e. cells suspected of containing the variant sequence characteristic of the disease, from one individual suspected of suffering from the disease, and a second sample comprising cells from a healthy tissue, i.e. cells which do not contain the variant sequence, from the same individual. The present methods in some embodiments allow a universal reference sample to be used.

Thus, frequently, it is desirable to test the DNA or RNA of an individual, such as a mammal, for example a human being. In that case the sample is a sample derived from said individual. Thus, the sample may for example comprise nucleic acids selected from the group consisting of DNA, mRNA, miRNA or any other RNA sequence. The sample may be derived from a body fluid sample for example a blood sample, a biopsy, a sample of hair, nails or the like or any other suitable sample. In embodiments of the invention, wherein the individual is suffering from cancer, the sample may be a sample of a cancer tumour removed from the individual by surgery, or a biopsy of said tumour. However, in embodiments of the invention wherein the individual is suffering from cancer, the sample may also be a blood sample, which typically may contain CTCs and cfDNA.

The sample may be processed in vitro prior to detection of the presence of the variant sequence. For example the sample may be subjected to one or more purification steps that may purify nucleic acids from the sample completely or partially. Furthermore, the sample may have been subjected to reverse transcription.

The sample may comprise a complex biological mixture of nucleic acid (RNA and DNA) and non-nucleic acids, for example an intact cell or a crude cell extract.

If the target DNA is double-stranded or otherwise has secondary and/or tertiary structure which may interfere with its detection, it may need to be heated prior to performing the methods of the invention. It may also be desirable in some cases to extract the nucleic acids from the complex biological samples prior to performing the amplification by any methods known in the art.

The sample may comprise a wide range of eukaryotic and prokaryotic cells, including protoplasts; or other biological materials that may harbour target deoxyribonucleic acids. The methods are thus applicable to tissue culture animal cells, animal cells (e.g., blood, serum, plasma, reticulocytes, lymphocytes, urine, bone marrow tissue, cerebrospinal fluid or any product prepared from blood or lymph) or any type of tissue biopsy (e.g. a muscle biopsy, a liver biopsy, a kidney biopsy, a bladder biopsy, a bone biopsy, a cartilage biopsy, a skin biopsy, a pancreas biopsy, a biopsy of the intestinal tract, a thymus biopsy, a mammal biopsy, an uterus biopsy, a testicular biopsy, an eye biopsy or a brain biopsy, homogenized in lysis buffer), plant cells or other cells sensitive to osmotic shock and cells of bacteria, yeasts, viruses, mycoplasmas, protozoa, rickettsia, fungi and other small microbial cells and the like. The assay and isolation procedures of the present invention are useful, for instance, for detecting non-pathogenic or pathogenic microorganisms of interest. By detecting the presence of a variant sequence in a biological sample, the presence of the microorganisms may be established.

In some embodiments, at least the first sample is obtained from an individual suffering from or suspected from a disease or a disorder which is characterised by the presence of a variant sequence as detailed above, in particular the presence of a variant microsatellite sequence or the presence of a mutant sequence. In some embodiments, the disease is a cancer or a genetic disorder, for example a pancreatic carcinoma, a gastric carcinoma, a bladder cancer, a prostate carcinoma, a lung cancer, a uterine carcinoma, a breast cancer, a hereditary non-polyposis colorectal cancer. In some embodiments, the disease is a genetic disorder such as Lynch syndrome, Huntington's disease (HD), dentatorubral and palidoluysian atrophy (DRPLA), spinobulbar and muscular atrophy (SBMA), myotonic dystrophy (DM), fragile X syndrome, FRAXE mental retardation and spinocerebellar ataxias (SCA) Bruton X-linked agammaglobulinemia (XLA), Bloom syndrome (BS), craniofrontonasal syndrome (CFNS) and idiopathic pulmonary fibrosis (IPF).

Amplification

The present methods require amplification of the target nucleic acid sequence comprising the NOI from the first sample and the second sample. This requires that a set of primers are provided. The set of primers consists of a first primer and a second primer, which together are capable of amplifying the target nucleic acid sequence comprising the NOI as is known in the art.

The set of primers is capable of priming amplification of the target nucleic acid sequence when used in e.g. a PCR, such as a real-time PCR, in the presence of said target nucleic acid sequence, and with reagents as otherwise known in the art. Such reagents are well known to the skilled person, and are for example described in Sambrook J et al. 2000. Molecular Cloning: A Laboratory Manual (Third Edition), Cold Spring Habor Laboratory Press. The reaction then generates an amplicon. The amplicon generated when the amplification is performed in the presence of the first sample is termed the first amplicon. The amplicon generated when the amplification is performed in the presence of the second (or reference) sample is termed the second amplicon or reference amplicon.

In order to render the set of primers specific for the target nucleic acid sequence, the primers comprise a sequence, which is identical to a stretch of the target nucleic acid sequence. In particular the 3′-end of the first and second primers may comprise a sequence of at least 15 nucleotides, which is identical to a stretch of the target nucleic acid sequence comprising the reference or variant sequence except for up to one mismatch.

The exact length of the sequence of the primers identical to a stretch of the target nucleic acid sequence may be adjusted in order to arrive at a primer having a melting temperature useful for PCR amplification. The melting temperature of a primer is dependent on several factors, but in particular on the GC content and the length. Because the primers should preferably be identical to a stretch of the target nucleic acid sequence comprising the variant sequence (or a sequence complementary to the variant sequence) there are restrictions to the specific sequence of the primer. Accordingly, the melting temperature may in particular be adjusted by adjusting the length of the primer. The skilled person is well capable of designing a primer with a suitable melting temperature and useful software to this end is publicly available.

Thus, the 3′-end of the primers may comprise a sequence of at least 15 nucleotides, for example at least 20 nucleotides, such as at least 25 nucleotides, for example in the range of 15 to 50 nucleotides, such as in the range of 20 to 40 nucleotides, which is identical to the target nucleic acid sequence comprising the variant sequence except for up to one mismatch.

In one embodiment of the invention, the first and/or the second primers consist of a sequence of at least 15 nucleotides, for example at least 20 nucleotides, such as at least 25 nucleotides, for example in the range of 15 to 50 nucleotides, such as in the range of 20 to 40 nucleotides, which is identical to the target nucleic acid sequence comprising the variant sequence except for up to one mismatch.

As mentioned above the sequence of the primers comprises or consists of a sequence identical (or complementary) to the target nucleic acid sequence comprising the variant sequence except for up to one mismatch. In some embodiments, there is one mismatch in one or both of the first and second primers, as this may even further improve the specificity of the assay. In particular said mismatch may be positioned at position 2, 3 or 4 from the 3′-end of the primers.

In some embodiments, it may be useful to design primers which hybridise to the target nucleic acid sequence outside the region corresponding to the variant sequence. For example, if the target nucleic acid sequence is a microsatellite, and the variant sequence differs from the reference sequence by the number of nucleotide repeats, the primers preferably hybridise upstream and downstream of the region consisting of tandem repeats, as the amplification otherwise may be unspecific and result in a variety of amplicons of different lengths.

The primers may comprise at least one hydrophobic nucleotide as described herein.

The step of amplification may be a PCR, such as a real-time PCR. In some embodiments, the amplification (e.g. PCR or real-time PCR) reaction is an asymmetric reaction. Asymmetric means that the reaction is directed towards amplifying more of one strand of the template than the other. It is performed by providing different amounts of the primers of a primer pair.

The first primer hybridises to the first strand of the target nucleic acid sequence, which comprises the NOI sequence. The second primer hybridises to the second strand of the target nucleic acid sequence, which is complementary to the NOI sequence. In a later step, the presence of variant sequence is detected by analysing the melting profile of the amplicons, which comprises a sequence identical to the NOI sequence, in the presence of a reporter probe, as described below. As is shown in the examples, analysis of the melting profiles is facilitated when the strand of an amplicon to which the reporter oligonucleotide can hybridise is present in greater quantity than the strand of the amplicon which is complementary to the reporter oligonucleotide. Thus, in preferred embodiments, the amplification step is asymmetric, i.e. it is performed with greater amount of the second primer than of the first primer. This directs the amplification towards generating more of the strand of the amplicon to which the reporter oligonucleotide hybridises.

Melting Analysis (Including HRM Analysis)

Melting analysis, in particular HRM (high-resolution melting) analysis, is based on the analysis of melting properties (transition profile from double- to single-stranded phase in particular) of formed heteroduplexed amplicons. The melting profile of the amplicons depends on their guanine-cytosine content, length, sequence, and heterozygosity. Changes in the nucleotide sequence give rise to the formation of the heteroduplexes that change the shape of the melting curve compared with the wild-type melting profile.

Once a first amplicon and a second amplicon have been obtained, e.g. through the use of an asymmetric amplification reaction, the method comprises the step of performing melting analysis, in particular HRM analysis, to obtain melt profiles (for example HRM profiles) for each amplicon. A first melt profile (or a first HRM profile) is obtained for the first amplicon, and a second melt profile (or a second HRM profile), also termed reference profile (or reference HRM profile) is obtained for the second amplicon. The melt profiles (or the HRM profiles) are then compared as described in detail below.

Each melt (e.g. HRM) profile is characterised by a melt curve. The first melt (e.g. HRM) profile of the first amplicon is characterised by a first melt curve, and the second (or reference) melt (e.g. HRM) profile of the second amplicon is characterised by a second (or reference) melt curve.

The melt (e.g. HRM) analysis of each amplicon involves hybridisation of a reporter oligonucleotide comprising at least one fluorophore and one quencher to one strand of each amplicon (having a sequence identical to the NOI sequence), e.g. the second strand. The reporter oligonucleotide is described in further detail below. After hybridisation, the signal emitted by the fluorophore is detected to obtain the first and second melt curves, as is otherwise known in the art. The method may further comprise the step of transforming the melt curves prior to further analysis. For example, the melt curves may be transformed to negative first derivative curves, which are then compared.

The first melt (e.g. HRM) profile and the second melt (e.g. HRM) profile are then compared. If the first sample contains a variant sequence, then the first melt curve (and optionally difference curves, and derivatives from the melt curves) will be different from the second melt curve (and optionally derivatives therefrom) corresponding to the reference sample. The detection of a difference between the first and the second melt (e.g. HRM) profiles thus indicates that the first sample contains a variant sequence.

A difference between melt (e.g. HRM) profiles may be a difference in the shape of melt curves or of difference curves, or in the shape of derivatives of the melt curves. For example, in some embodiments the first melt curve has a different shape than the reference melt curve. Typically, derivatives of melt curves are normal curves (or bell-shaped curves) with a maximum value. The slope of the first melt curve may be less “sharp” than the slope of the second curve, which causes a change in the shape of the first melt curve compared to the second melt curve. The slope of the derivative of the first melt curve may be “sharper” than the slope of the derivative of the second curve, for example the absolute value of the slope is greater for the derivative of the first melt curve than the absolute value of the slope of the derivative for the second melt curve, at least in its inflection point(s). In order to determine a difference between melt (e.g. HRM) profiles, the analysis may comprise the step of obtaining a difference curve, where one of the melt curves is set as reference and subtracted from the other melt curve, preferably wherein the melt curve of the reference sample is set as reference and subtracted from the melt curve of the variant sample.

The two curves (or difference curves or derivatives) may at each temperature T be distant from one another by a distance D_{T}. The distance D_{T} is determined for each temperature, and the absolute value of the greatest distance maxD_{T} indicates a difference between two curves if it is greater than a pre-determined threshold.

The skilled person will know how to determine a suitable value for the threshold. The threshold is a D_{T} value which allows to discriminate between subjects displaying microsatellite instability and normal subjects. The D_{T} value is a difference, for example a negative difference, or a positive difference, between the melt profile obtained for the reference (or wild type) and the melt profile obtained for the variant sequence (s). In order to determine a suitable threshold value, the melt profiles of a number of subjects (typically 100-500 patients), for which it is known whether they have microsatellite instability or not, are established. Using these melt profiles, the threshold is established as the value which allows for the desired or suitable discrimination between wild type and variant sequences.

The difference between the analysed melt profiles is thus calculated and compared to the threshold; this difference can thus be a numerical difference, which can be compared to a numerical threshold. If this difference between the analysed melt profiles, e.g. the HRM profiles, is greater than the threshold, the sample from which the first melt profile is determined is classified as comprising the variant sequence. When the target nucleic acid sequence is a microsatellite, the subject from which the sample was obtained is thus considered as having microsatellite instability. If the difference between the analysed melt profiles, e.g. the HRM profiles, is lower than the threshold, the sample is classified as comprising the wild type sequence. When the target nucleic acid sequence is a microsatellite, the subject from which the sample was obtained is thus considered as not having microsatellite instability.

An alternative is to determine the area between the curves. If the area is greater than a predetermined threshold, the first and the second melt curves are considered different. Here too, the skilled person will know how to set a value for the threshold.

Other ways of characterising a difference between two melt curves or their difference curves or their derivatives will be apparent to the skilled person.

Any of the above differences between the first and second melt curves or between their difference curves or derivatives of the first and second melt curves indicates a difference between the first and second HRM profiles, and thus indicates the presence of a variant sequence in the first sample.

In some embodiments, the first and second melt curves and/or their derivatives and/or difference curves are normalised. For example, close to or at the lowest temperature for which melting is measured, such as within 0.5 to 20° C. from the lowest temperature, the curve is normalised to a first value, and close to or at the highest temperature for which melting is measured, such as within 0.5 to 20° C. from the highest temperature, the curve is normalised to a second value. For instance, the first value is 1 and the second value is 0.

In order to render the differences between curves more visible, the melt analysis, e.g. the HRM analysis, may comprise a step of standard normalisation, which corresponds to aligning the curves in the y axis based on the mean dye intensity in the initial and final normalisation areas.

One can also apply bilinear normalisation to each curve (melt curve, difference graph or derivative). A first linear function is fitted to initial dye intensity, and used as the top end of the final corrected scale. A second linear function is fitted to final dye intensity, and used as the bottom end of the corrected scale.

The melt analysis, e.g. the HRM analysis, may also, or alternatively, comprise a step of applying a temperature adjustment (temperature shift) at a relative fluorescent unit (RFU) to the curves (melt curves or difference curves). Such steps may also reduce the risk of false positives. In preferred embodiments, the melt analysis comprises at least the step of applying a temperature adjustment at a relative fluorescent unit to the melt curves.

Accordingly, in some embodiments, the step of comparing the first profile to the reference profile comprises or consists of the steps of:
i) aligning the first and the second melt curves at a given fluorescent intensity along the temperature axis, thereby nullifying differences in melting temperatures between the first and the second melt curves at said fluorescent intensity;
ii) determining the difference in the signal emitted by the fluorophore between the first and the second melt curves; and
iii) comparing the difference determined in ii) to a threshold value, wherein a difference greater than the threshold indicates that the first sample comprises a variant sequence and a difference smaller than the threshold indicates that the first sample comprises the reference sequence.

The threshold value is determined as explained herein above. The difference is a numerical difference, which is compared to the numerical threshold value as explained above. Thus in step iii), if the threshold is a positive threshold, a difference greater than said threshold indicates that the first sample comprises a variant sequence. If the threshold is a negative threshold, a difference smaller than said threshold indicates that the first sample comprises a variant sequence.

In some embodiments, the methods disclosed herein do not comprise a step of determining the exact length and/or sequence of the variant sequence. This is because it often is not required to determine the exact length and/or sequence of variant sequences to determine that the individual in which the variant sequence is found suffers from a disease or disorder as described herein. Rather, it often suffices to establish that there is a difference—the exact nature of the difference is not always relevant. In preferred embodiments, the methods disclosed herein do not comprise a step of determining the exact length and/or sequence of the variant sequence. This is because detection of a variant sequence can be sufficient to select samples for further analysis; it is typically not necessary to determine the exact length and/or sequence of the variant sequences.

### Reporter Oligonucleotide

The present methods require a reporter oligonucleotide to perform the HRM analysis. The reporter oligonucleotide comprises at least a first fluorophore and at least a first quencher. These are helpful for melt analysis such as HRM analysis. Preferably, the reporter oligonucleotide comprises a first fluorophore at least in its 5′-end or at least in its 3′-end or within 4 nucleotides from the 5′-end or the 3′-end. The reporter oligonucleotide preferably comprises a first quencher at least in its 5′-end or at least in its 3′-end or within 4 nucleotides from the 5′-end or the 3′-end. Thus, the fluorophore and quencher may be located in the 5′-end or 3′-end or within 4 nucleotides from the 5′-end or the 3′-end but are not necessarily located at the last nucleotide of the reporter oligonucleotide. Preferably, if the first fluorophore is located at the 5′-end or within 4 nucleotides from the 5′-end, the first quencher is not located at the 5′-end or within 4 nucleotides from the 5′-end. Instead, it is located at the 3′-end or within 4 nucleotides from the 3′-end or in an internal region of the reporter. Conversely, if the first fluorophore is located at the 3′-end or within 4 nucleotides from the 3′-end, the first quencher is not located at the 3′-end or within 4 nucleotides from the 3′-end. Instead, it is located at the 5′-end or within 4 nucleotides from the 5′-end or in an internal region of the reporter. The term internal region here is the region of the reporter oligonucleotide which does not include the 5 terminal nucleotides in each end. Preferably, the first fluorophore and the first quencher are not in close vicinity to each other.

Useful fluorophores and quenchers are readily available to the skilled person, who will have no difficulty selecting them to perform the present methods.

The reporter oligonucleotide is used for the melt analysis (or HRM analysis). As such, the sequence of the reporter oligonucleotide is submitted to some constraints, which depend on the sequence to which it is to hybridise and detect. The reporter oligonucleotide comprises a hybridisation sequence H, which is for example identical to the NOI, and which hybridises to the strand complementary to the NOI. In other words, the reporter oligonucleotide hybridises to the strand of the first and second amplicons, which comprises the NOI. In embodiments where the NOI comprises repeats, the reporter oligonucleotide thus also comprises repeats, i.e. the hybridisation sequence H also comprises repeats, as further detailed below.

The reporter oligonucleotides may, in addition to the sequence which is identical to the NOI, further comprise additional nucleotides in the hybridisation sequence. For example, as shown in the examples below, this may be particularly relevant in the cases where the target nucleic acid is a microsatellite or if the variant sequence is expected to comprise an insertion. If the microsatellite has M tandem repeats having a total length of n nucleotides in the reference sequence, the hybridisation sequence of the reporter oligonucleotide preferably has M″ tandem repeats, wherein M″≥M+1, preferably M″≥M+1 or M″≥M+2. If the tandem repeat is a mononucleotide repeat, the hybridisation sequence then has a length of n″ nucleotides, where n″≥n+1, preferably n″≥n+1 or n″≥n+2. In other words, the hybridisation sequence may comprise at least one or two additional nucleotides. This allows more sensitive discrimination between the longer variant sequence and the reference sequence.

In some embodiments, in particular where the NOI is a microsatellite, the hybridisation sequence of the reporter oligonucleotide preferably comprises a sequence which consists of repeats identical or complementary to the repeats of the NOI, and may advantageously also comprise a terminal sequence, herein termed helper sequence, which hybridises to the first and second amplicons just upstream or just downstream to the repeats when the hybridisation sequence is hybridised thereto. In other words, the hybridisation sequence of the reporter oligonucleotide preferably comprises repeats, and additionally comprises 1 to 10 nucleotides, such as 1, 2, 3, 4, 5, 6, 7, 8, 9 or 10 nucleotides, which hybridise to the NOI or its complementary strand just upstream or downstream of the repetitive sequence. The helper sequence, when hybridised to the first and second amplicons, thus allows the hybridisation sequence to hybridise to said repetitive sequence. The helper sequence will often have a higher affinity for its complementary sequence than the hybridisation sequence for its complementary sequence. In other words, the helper sequence will often hybridise faster to its complementary sequence than the hybridisation sequence will hybridise to its complementary sequence.

The helper sequence may be 3′-terminal or 5′-terminal. Preferably, the reporter oligonucleotide comprises a 5′-terminal helper sequence which hybridises to the first and second amplicons just upstream or just downstream of the repeats when the hybridisation sequence is hybridised thereto. The helper sequence facilitates discrimination between the melting profiles, as shown in example 13. A helper sequence is termed strong if it leads to an increase in the Tm of the hybridisation sequence, when the helper sequence is hybridised to the first or second amplicon, which is high. The skilled person knows well that hybridisation between two sequences is stronger if the sequences comprise a higher proportion of G/C compared to two hybridised sequences comprising a higher proportion of A/T; hence, a helper sequence comprising a higher proportion of G/C than A/T will be stronger than a helper sequence comprising a lower proportion of G/C than A/T. It is also known that longer sequences give a stronger hybridisation than shorter sequences, hence the strength of a helper sequence may be adjusted by increasing the length that hybridises to the first and/or second amplicons. Increasing the strength of a helper sequence may be a good way of facilitating discrimination between a variant sequence and a reference sequence. Preferably, the helper sequence does not comprise repeats, even in embodiments where the NOI comprises repeats.

In some embodiments, particularly where the target nucleic acid sequence is a microsatellite, the helper sequence increases the Tm of the hybridisation sequence by at least 5° C., such as at least 6° C., such as at least 7° C., such as at least 8° C., such as at least 9° C., such as at least 10° C., such as at least 11° C., such as at least 12° C., such as at least 13° C., such as at least 14° C., such as at least 15° C., or more, compared to the Tm of the same hybridisation sequence without the helper sequence. The Tm of the hybridisation sequence comprising the helper sequence may thus be between 5 and 25° C. higher, for example between 10 and 20° C. higher, such as between 12.5 and 17.5° C. higher than the Tm of the hybridisation sequence without the helper sequence.

The reporter oligonucleotide may comprise a second quencher. Preferably, the second quencher is in a non-terminal region, i.e. located in the internal region of the reporter oligonucleotide. In other words, the second quencher is not located at the 5′-end or at the 3′-end or within 4 nucleotides from the 5′- or 3′-end. The inclusion of a second quencher may in some embodiments facilitate discrimination between variant sequence and reference sequence.

While any reporter oligonucleotide may be used which can enable melting analysis, in particular HRM analysis, i.e. any reporter oligonucleotide with the features described above, some reporter oligonucleotides are particularly advantageous. The inclusion of hydrophobic nucleotides at particular positions of the reporter oligonucleotide are of particular interest. As can be seen from the examples, reporter oligonucleotides comprising such hydrophobic nucleotides increase sensitivity of the method.

Thus, in some embodiments, in addition to the features described above, the reporter oligonucleotide comprises at least one hydrophobic nucleotide positioned at its 5′-end or within 10 nucleotides from the 5′-end, and/or the reporter oligonucleotide comprises at least one hydrophobic nucleotide positioned at its 3′-end or within 10 nucleotides from the 3′-end.

The hydrophobic nucleotide has the structure

X—Y-Q

wherein

X is a nucleotide or nucleotide analogue or a backbone monomer unit capable of being incorporated into the backbone of a nucleic acid,

Q is an intercalator, which is not taking part in Watson-Crick hydrogen bonding; and

Y is a linker moiety linking said nucleotide or nucleotide analogue or backbone monomer unit and said intercalator.

The backbone monomer unit X may be any of the backbone monomer units described herein below in the section “Backbone monomer unit”.

The intercalator Q may be any of the intercalators described herein below in the section “Intercalator”.

Hydrophobic nucleotides useful in the context of the present disclosure are described in detail in international patent application WO 2017/045689, in particular in the section entitled “Hydrophobic nucleotides” on p. 30 I. 2 to I. 25.

In some embodiments, the reporter oligonucleotide has the following general structure

5′-(N)ₐ—Z—(N)_{d}—Z—(N)ₑ—Z—(N)_{b}-3′
wherein
N is any nucleotide or nucleotide analogue; and
Z is a hydrophobic nucleotide as defined in item 1; and
the total number of nucleotides or nucleotide analogues is at least 10; and
a and b individually are integers in the range of 0 to 4; and
d and e individually are integers in the range of 1 to 19; and
a+b+d+e at least 10; and
   (N)ₐ—(N)_{d}—(N)ₑ—(N)_{b} is identical to the reference sequence.

In other embodiments, the reporter oligonucleotide has the following general structure

5′-(N)ₐ—Z—(N)_{f}—Z—(N)_{g}—Z—(N)ₕ—Z—(N)_{b}-3′
wherein
N is any nucleotide or nucleotide analogue; and
Z is a hydrophobic nucleotide as defined in claim 1; and
a and b individually are integers in the range of 0 to 4; and
f, g and h individually are integers in the range of 1 to 18; and
a+b+f+g+h is at least 10 and at the most 50; and
(N)ₐ—(N)_{f}—(N)_{g}—(N)ₕ—(N)_{b} is identical to a stretch of the target nucleic acid sequence comprising the reference sequence; or
   has the following general structure

5′-(N)ₐ—Z—(N)ᵢ—Z—(N)ⱼ—Z—(N)ₖ—Z—(N)ₗ—Z—(N)_{b}-3′
wherein
N is any nucleotide or nucleotide analogue; and
Z is a hydrophobic nucleotide as defined in claim 1; and
a and b individually are integers in the range from 0 to 4; and
j, k and I individually are integers in the range from 1 to 17; and
a+b+i+j+k+l is at least 10 and at the most 50; and
(N)ₐ—(N)ᵢ—(N)ⱼ—(N)ₖ—(N)ᵢ—(N)_{b} is identical to a stretch of the target nucleic acid sequence comprising the reference sequence; or
   has the following general structure

5′-(N)ₐ—Z—(N)ₘ—Z—(N)ₙ—Z—(N)ₒ—Z—(N)ₚ—Z—(N)_{q}—Z—(N)_{b}-3′
wherein

N is any nucleotide or nucleotide analogue; and

Z is a hydrophobic nucleotide as defined in claim 1; and

a and b individually are integers in the range of 0 to 4; and

m, n, o, p and q individually are integers in the range of 1 to 16; and

a+b+m+n+o+p+q is at least 10 and at the most 50; and

(N)ₐ—(N)ₘ—(N)ₙ—(N)ₒ—(N)ₚ—Z—(N)_{q}—(N)_{b} is identical to a stretch of the target nucleic acid sequence comprising the reference sequence.

In some embodiments, the reporter oligonucleotide is for detection of a microsatellite. In such embodiments, the hybridization sequence encompasses at least the tandem repeats. Preferably, the hybridization sequence also comprises a helper sequence consisting of between 1 and 20 nucleotides immediately upstream or downstream of the tandem repeats, such as 1, 2, 3, 4, 5, 6, 7, 8, 9, 10, 11, 12, 13, 14, 15, 16, 17, 18, 19 or 20 nucleotides immediately upstream or downstream of the tandem repeats, preferably wherein at least one hydrophobic nucleotide as described herein has been inserted. The helper sequence may consist of at least one, such as 1, for example 2, such as 3, for example 4, such as 5, for example 6, such as 7, for example 8, such as 9, for example 10, such as from 10 to 20, for example from 20 to 50, such as more than 50 nucleotides. For example, the helper sequence consists of 15 nucleotides or more. Thus in some embodiments, the helper sequence consists of 5, 6, 7, 8, 9, 10, 11, 12, 13, 14, 15, 16, 17, 19, 20 nucleotides or more, such as 25, 30, 35, 40, 45 or 50 nucleotides or more. For example, the helper sequence consists of 20, 21, 22, 23, 24, 25, 26, 27, 28, 29 or 30 nucleotides or more. For example, the helper sequence consists of 7, 8, 9, 10, 11, 12, 13, 14 or 15 nucleotides or more, for example 9 nucleotides.

In embodiments where the reporter oligonucleotide is to be used for detection of a microsatellite, such as BAT25, BAT26, NR21, NR22, NR24 or MONO27 as described above.

Intercalator

The term intercalator in the context of the present methods refers to any molecular moiety comprising at least one essentially flat conjugated system, which is capable of co-stacking with nucleobases of a nucleic acid. Preferably an intercalator essentially consists of at least one essentially flat conjugated system, which is capable of co-stacking with nucleobases of a nucleic acid.

An intercalator comprises at least one π (phi) electron system, which according to the present invention can interact with other molecules comprising a π electron system. These interactions can contribute in a positive or negative manner to the hydrophobic interactions of said intercalators. Hunter and Sanders (1990) J. Am Chem. Soc. 112: 5525-5534, have proposed a range of different orientations and conditions where two π electron systems can interact positively with each other.

Preferably, the intercalator comprises a chemical group selected from the group consisting of polyaromates and heteropolyaromates an even more preferably the intercalator essentially consists of a polyaromate or a heteropolyaromate. Most preferably the intercalator is selected from the group consisting of polyaromates and heteropolyaromates.

Polyaromates or heteropolyaromates according to the present invention may consist of any suitable number of rings, such as 1, for example 2, such as 3, for example 4, such as 5, for example 6, such as 7, for example 8, such as more than 8. Furthermore polyaromates or heteropolyaromates may be substituted with one or more selected from the group consisting of hydroxyl, bromo, fluoro, chloro, iodo, mercapto, thio, cyano, alkylthio, heterocycle, aryl, heteroaryl, carboxyl, carboalkoyl, alkyl, alkenyl, alkynyl, nitro, amino, alkoxyl, carbonyl and amido.

Accordingly, an intercalator Q may for example be an intercalator selected from the group consisting of phenanthroline, phenazine, phenanthridine, anthraquinone, pyrene, anthracene, napthene, phenanthrene, picene, chrysene, naphtacene, acridones, benzanthracenes, stilbenes, oxalo-pyridocarbazoles, azidobenzenes, porphyrins, psoralens and any of the aforementioned intercalators substituted with one or more selected from the group consisting of hydroxyl, bromo, fluoro, chloro, iodo, mercapto, thio, cyano, alkylthio, heterocycle, aryl, heteroaryl, carboxyl, carboalkoyl, alkyl, alkenyl, alkynyl, nitro, amino, alkoxyl and/or amido.

Preferably, the intercalator is selected from the group consisting of phenanthroline, phenazine, phenanthridine, anthraquinone, pyrene, anthracene, napthene, phenanthrene, picene, chrysene, naphtacene, acridones, benzanthracenes, stilbenes, oxalo-pyridocarbazoles, azidobenzenes, porphyrins and psoralens.

In a preferred embodiment, the intercalator is selected from the group consisting of phenanthroline, phenazine, phenanthridine, anthraquinone, pyrene, anthracene, phenanthrene, chrysene, naphtacene, benzanthracenes, stilbenes and porphyrins

In another preferred embodiment the intercalator comprises pyrene or pyrido[3′,2′:4,5]thieno[3,2-d]pyrimidin-4(1H)-one or 7,9-dimethyl-pyrido[3′,2′,4,5]thieno[3,2-d]pyrimidin-4(3H)-one. The intercalator may also consist of pyrene or pyrido[3′,2′:4,5]thieno[3,2-d]pyrimidin-4(1H)-one, or 7,9-dimethyl-pyrido[3′,2′,4,5]thieno[3,2-d]pyrimidin-4(3H)-one.

The hydrophobic nucleotide may comprise other intercalators, in particular the intercalators described in WO 2017/045689 in the section entitled “Intercalator” on p. 30 I. 26 to p. 40 I. 4, or in international patent application WO 03/052132 in the section “intercalator” on p. 46, I. 10 to p. 54, I. 13.

Backbone Monomer Unit

X may also be a backbone monomer unit capable of being incorporated into the backbone of a nucleic acid or nucleic acid analogue. The backbone monomer unit of a nucleotide or a nucleotide analogue herein refers to the part of the nucleotide, which is involved in incorporation into the backbone of a nucleic acid or a nucleic acid analogue. The backbone monomer unit (X) is preferably covalently linked to a linker (Y), which is covalently linked to the intercalator. Any suitable backbone monomer unit may be employed for incorporating intercalator into the oligonucleotide analogues. Any sort of linker linking said backbone monomer unit and said intercalator could also be employed. In addition, the backbone monomer unit may comprise one or more leaving groups, protecting groups and/or reactive groups, which may be removed or changed in any way during synthesis or subsequent to synthesis of an oligonucleotide or oligonucleotide analogue comprising said backbone monomer unit.

The backbone monomer unit may be any suitable backbone monomer unit. In one embodiment, the backbone monomer unit may for example be selected from the group consisting of the backbone monomer units of DNA, RNA, PNA, HNA, XNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, α-L-RNA or α-D-RNA, β-D-RNA and mixtures thereof and hybrids thereof, as well as phosphorous atom modifications thereof, such as but not limited to phosphorothioates, methyl phospholates, phosphoramidiates, phosphorodithiates, phosphoroselenoates, phosphotriesters and phosphoboranoates. In addition non-phosphorous containing compounds may be used for linking to nucleotides such as but not limited to methyliminomethyl, formacetate, thioformacetate and linking groups comprising amides.

A range of backbone monomer units are described in international patent application WO 2017/045689, in the section entitled “Backbone monomer unit” on p. 40 I. 5 to p. 56 I. 3 and in international patent application WO03/052132 in the section “Backbone monomer unit” on p. 24, I. 27 to p. 43, I. 14. These also describe a range of different backbone monomer units of nucleotides and nucleotide analogues useful with the present invention, and how they are connected to the nucleobases via linkers that are attached at one or two positions of the backbone monomer unit.

Linker

The linker of an intercalator nucleotide is a moiety connecting the intercalator and the backbone monomer of said hydrophobic nucleotide, preferably covalently linking said intercalator and the backbone monomer unit. The linker may comprise one or more atom(s) or bond(s) between atoms.

By the definitions of backbone and intercalator given herein above, the linker is the shortest path linking the backbone and the intercalators. If the intercalator is linked directly to the backbone, the linker is a bond.

The linker usually consists of a chain of atoms or a branched chain of atoms. Chains can be saturated as well as unsaturated. The linker may also be a ring structure with or without conjugated bonds.

Useful linkers are described in detail in in international patent application WO 2017/045689, in particular in the section entitled “Linker” on p. 56 I. 5 to p. 59 I. 10 and in WO03/052132 in the section “Linker” on p. 54, I. 15 to p. 58, I. 7.

Kit of Parts

Herein is also provided a kit of parts for detecting the presence of a variant sequence in a target nucleic acid sequence consisting of two strands and comprising nucleotide(s) of interest (NOI) preferably comprising repeats, wherein said target nucleic acid sequence consists of a variant sequence or of a reference sequence, said kit of parts comprising:
a) a reporter oligonucleotide comprising a first fluorophore, preferably in its 5′-end or within 4 nucleotides from the 5′-end, and a first quencher, preferably in its 3′-end or within 4 nucleotides from the 3′-end,
   wherein the reporter oligonucleotide is a sequence of in the range of 10 to 50 nucleotides, preferably in the range of 15 to 50 nucleotides, into which in the range of 2 to 10 hydrophobic nucleotides have been inserted and
   wherein the reporter oligonucleotide comprises a hybridization sequence H, and
   wherein the hybridisation sequence is identical to a consecutive stretch of the sequence of a first strand of the target nucleic acid, and wherein the hybridisation sequence is complementary to a consecutive stretch of the sequence of a second strand of the target nucleic acid;
   and wherein the hybridisation sequence of the reporter oligonucleotide comprises or consists of a repetitive sequence and at least one helper sequence in its 5′-end and/or in its 3′-end, wherein said helper sequence does not comprise repeats, and can hybridise to the first and second amplicons when the hybridisation sequence is hybridized thereto; and
b) a set of primers consisting of a first primer and a second primer, wherein the set of primers together are capable of amplifying the target nucleic acid sequence.

Herein is also provided a kit of parts comprising:
a) a reporter oligonucleotide, which may be any of the reporter oligonucleotides described herein below in the section “Reporter oligonucleotide”
b) a set of primers consisting of a first primer and a second primer, which may be any of the sets of primers described herein above in the section “Amplification”.

The kit-of-parts is in particular useful for performing the present methods.

Thus is provided herein a kit of parts for detecting the presence of a variant sequence in a target nucleic acid sequence consisting of two strands and comprising nucleotide(s) of interest (NOI), wherein said target nucleic acid sequence consists of a variant sequence or of a reference sequence, said kit of parts comprising:
a) a reporter oligonucleotide comprising a first fluorophore, preferably in its 5′-end or within 4 nucleotides from the 5′-end, and a first quencher, preferably in its 3′-end or within 4 nucleotides from the 3′-end,
   wherein the reporter oligonucleotide is a sequence of in the range of 10 to 50 nucleotides into which in the range of 2 to 10 hydrophobic nucleotides have been inserted and wherein the reporter oligonucleotide comprises a hybridization sequence H,
wherein
at least one hydrophobic nucleotide is positioned at the 5′-end or within 10 nucleotides from the 5′-end of the reporter oligonucleotide; and/or
at least one hydrophobic nucleotide is positioned at the 3′-end or within 10 nucleotides from the 3′-end of the reporter oligonucleotide; and
wherein the hydrophobic nucleotide has the structure

X—Y-Q
wherein
   X is a nucleotide or nucleotide analogue or a backbone monomer unit capable of being incorporated into the backbone of a nucleic acid or nucleic acid analogue,
   Q is an intercalator which is not taking part in Watson-Crick hydrogen bonding; and
   Y is a linker moiety linking said nucleotide or nucleotide analogue or backbone monomer unit and said intercalator; and
   wherein the hybridisation sequence is identical to a consecutive stretch of the sequence of the first strand of the target nucleic acid comprising the reference sequence, and wherein the hybridisation sequence is complementary to a consecutive stretch of the sequence of the second strand of the target nucleic acid; and
b) a set of primers consisting of a first primer and a second primer, wherein the set of primers together are capable of amplifying the target nucleic acid.

In addition to said reporter oligonucleotide and said set of primers, the kit-of-parts may also comprise additional components. For example, the kit-of-parts may further comprise PCR reagents. The kit-of-parts may also comprise a detection probe, such as a probe allowing for real time detection of the generation of a PCR product.

The above kits are particularly useful for performing the methods described herein, in particular for detecting variant sequences of a target nucleic acid sequence preferably comprising repeats, for example microsatellites. In particular, microsatellites where the wild type or reference sequence is 15 nucleotides or more, as detailed herein above.

Reporter Oligonucleotide

As detailed herein above, the present methods require a reporter oligonucleotide to perform the melting analysis, for example to perform HRM analysis. Without being bound by theory, the inventors have found that reporter oligonucleotides comprising hydrophobic nucleotides are particularly useful for performing the present methods, as they may increase sensitivity of the assay. Such reporter oligonucleotides are thus also disclosed herein.

Herein is also provided a reporter oligonucleotide which can hybridise to one strand of a target nucleic acid consisting of two strands and comprising nucleotide(s) of interest (NOI) preferably comprising repeats, said reporter oligonucleotide comprising a first fluorophore, preferably in its 5′-end or within 4 nucleotides from the 5′-end, and a first quencher, preferably in its 3′-end or within 4 nucleotides from the 3′-end, wherein the reporter oligonucleotide is a sequence of in the range of 10 to 50, preferably in the range of 15 to 50 nucleotides, into which in the range of 2 to 10 hydrophobic nucleotides have been inserted and wherein the reporter oligonucleotide comprises a hybridization sequence H,

wherein the hybridisation sequence is identical to a consecutive stretch of the sequence of a first strand of the target nucleic acid, and wherein the hybridisation sequence is complementary to a consecutive stretch of the sequence of a second strand of the target nucleic acid,
and wherein the hybridisation sequence of the reporter oligonucleotide comprises or consists of a repetitive sequence and at least one helper sequence in its 5′-end and/or in its 3′-end, wherein said helper sequence does not comprise repeats, and can hybridise to the second strand of the first and second amplicons when the hybridisation sequence is hybridized thereto.

Herein is also provided a reporter oligonucleotide comprising a first fluorophore, preferably in its 5′-end or within 4 nucleotides from the 5′-end, and a first quencher, preferably in its 3′-end or within 4 nucleotides from the 3′-end, wherein the reporter oligonucleotide is a sequence of in the range of 10 to 50 nucleotides into which in the range of 2 to 10 hydrophobic nucleotides have been inserted and wherein the reporter oligonucleotide comprises a hybridization sequence H,
wherein
at least one hydrophobic nucleotide is positioned at the 5′-end or within 10 nucleotides from the 5′-end of the reporter oligonucleotide; and/or
at least one hydrophobic nucleotide is positioned at the 3′-end or within 10 nucleotides from the 3′-end of the reporter oligonucleotide; and
wherein the hydrophobic nucleotide has the structure

X—Y-Q
wherein
   X is a nucleotide or nucleotide analogue or a backbone monomer unit capable of being incorporated into the backbone of a nucleic acid or nucleic acid analogue,
   Q is an intercalator which is not taking part in Watson-Crick hydrogen bonding; and
   Y is a linker moiety linking said nucleotide or nucleotide analogue or backbone monomer unit and said intercalator; and
      wherein the hybridisation sequence is identical to a consecutive stretch of the sequence of a first strand of a target nucleic acid, and wherein the hybridisation sequence is complementary to a consecutive stretch of the sequence of a second strand of the target nucleic acid.

The reporter oligonucleotide may comprise a second quencher. Preferably, the second quencher is in a non-terminal region, i.e. located in the internal region of the reporter oligonucleotide. In other words, the second quencher is not located at the 5′-end or at the 3′-end or within 4 nucleotides from the 5′- or 3′-end.

The backbone monomer unit X may be any of the backbone monomer units described herein above in the section “Backbone monomer unit”.

The intercalator Q may be any of the intercalators described herein above in the section “Intercalator”.

Hydrophobic nucleotides useful in the context of the present disclosure are described in detail in international patent application WO 2017/045689, in particular in the section entitled “Hydrophobic nucleotides” on p. 30 I. 2 to I. 25.

In some embodiments, the reporter oligonucleotide has the following general structure

5′-(N)ₐ—Z—(N)_{d}—Z—(N)ₑ—Z—(N)_{b}-3′
wherein
N is any nucleotide or nucleotide analogue; and
Z is a hydrophobic nucleotide as defined in item 1; and
the total number of nucleotides or nucleotide analogues is at least 10; and
a and b individually are integers in the range of 0 to 4; and
d and e individually are integers in the range of 1 to 19; and
a+b+d+e at least 10; and
   (N)ₐ—(N)_{d}—(N)ₑ—(N)_{b} is identical to the reference sequence.

In other embodiments, the reporter oligonucleotide has the following general structure

5′-(N)ₐ—Z—(N)_{f}Z—(N)_{g}—Z—(N)ₕ—Z—(N)_{b}-3′
wherein
N is any nucleotide or nucleotide analogue; and
Z is a hydrophobic nucleotide as defined in claim 1; and
a and b individually are integers in the range of 0 to 4; and
f, g and h individually are integers in the range of 1 to 18; and
a+b+f+g+h is at least 10 and at the most 50; and
(N)ₐ—(N)_{f}—(N)_{g}—(N)ₕ—(N)_{b} is identical to a stretch of the target nucleic acid sequence comprising the reference sequence; or
   has the following general structure

5′-(N)ₐ—Z—(N)ᵢ—Z—(N)ⱼ—Z—(N)ₖ—Z—(N)ₗ—Z—(N)_{b}-3′
wherein
N is any nucleotide or nucleotide analogue; and
Z is a hydrophobic nucleotide as defined in claim 1; and
a and b individually are integers in the range from 0 to 4; and
j, k and I individually are integers in the range from 1 to 17; and
a+b+i+j+k+l is at least 10 and at the most 50; and
(N)ₐ—(N)ᵢ—(N)ⱼ—(N)ₖ—(N)ₗ—(N)_{b} is identical to a stretch of the target nucleic acid sequence comprising the reference sequence; or
   has the following general structure

5′-(N)ₐ—Z—(N)ₘ—Z—(N)ₙ—Z—(N)ₒ—Z—(N)ₚ—Z—(N)_{q}—Z—(N)_{b}-3′
wherein
   N is any nucleotide or nucleotide analogue; and
   Z is a hydrophobic nucleotide as defined in claim 1; and
   a and b individually are integers in the range of 0 to 4; and
   m, n, o, p and q individually are integers in the range of 1 to 16; and
   a+b+m+n+o+p+q is at least 10 and at the most 50; and
   (N)ₐ—(N)ₘ—(N)ₙ—(N)ₒ—(N)ₚ—Z—(N)_{q}—(N)_{b} is identical to a stretch of the target nucleic acid sequence comprising the reference sequence.

The reporter oligonucleotide may comprise an overhang, i.e. it may comprise nucleotides in one of its ends which do not hybridise to the target nucleic acid sequence. The overhang may be 3′-terminal or 5′-terminal. Preferably, the reporter oligonucleotide comprises a 5′-terminal sequence which forms a 5′-overhang relative to the strand of the first and second amplicons to which the hybridisation sequence can hybridise.

The reporter oligonucleotide comprises at least a first fluorophore and at least a first quencher. These are helpful for melting analysis and/or HRM analysis. Preferably, the reporter oligonucleotide comprises a first fluorophore at least in its 5′-end or at least in its 3′-end or within 4 nucleotides from the 5′-end or the 3′-end. The reporter oligonucleotide preferably comprises a first quencher at least in its 5′-end or at least in its 3′-end or within 4 nucleotides from the 5′-end or the 3′-end. Thus, the fluorophore and quencher may be located in the 5′-end or 3′-end or within 4 nucleotides from the 5′-end or the 3′-end but are not necessarily located at the last nucleotide of the reporter oligonucleotide. Preferably, if the first fluorophore is located at the 5′-end or within 4 nucleotides from the 5′-end, the first quencher is not located at the 5′-end or within 4 nucleotides from the 5′-end. Instead, it is located at the 3′-end or within 4 nucleotides from the 3′-end or in an internal region of the reporter. Conversely, if the first fluorophore is located at the 3′-end or within 4 nucleotides from the 3′-end, the first quencher is not located at the 3′-end or within 4 nucleotides from the 3′-end. Instead, it is located at the 5′-end or within 4 nucleotides from the 5′-end or in an internal region of the reporter. The term internal region here is the region of the reporter oligonucleotide which does not include the 5 terminal nucleotides in each end. Preferably, the first fluorophore and the first quencher are not in close vicinity to each other.

Useful fluorophores and quenchers are readily available to the skilled person, who will have no difficulty selecting them to perform the present methods.

The reporter oligonucleotide is used for performing melting analysis, for example HRM analysis. As such, the sequence of the reporter oligonucleotide is submitted to some constraints, which depend on the sequence to which it is to hybridise and detect. The reporter oligonucleotide comprises a hybridisation sequence H, which is e.g. identical to the NOI, and which hybridises to the strand complementary to the NOI. In embodiments where the NOI comprises repeats, the reporter oligonucleotide thus also comprises repeats, i.e. the hybridisation sequence H also comprises repeats, as further detailed below.

The reporter oligonucleotides may, in addition to the sequence which is identical to the NOI, further comprise additional nucleotides in the hybridisation sequence. For example, as shown in the examples below, this may be particularly relevant in the cases where the target nucleic acid is a microsatellite. If the microsatellite has M tandem repeats having a total length of n nucleotides in the reference sequence, the hybridisation sequence of the reporter oligonucleotide preferably has M″ tandem repeats, wherein M″≥M+1, preferably M″≥M+1 or M″≥M+2. If the tandem repeat is a mononucleotide repeat, the hybridisation sequence then has a length of n″ nucleotides, where n″≥n+1, preferably n″≥n+1 or n″≥n+2. In other words, the hybridisation sequence may comprise at least one or two additional nucleotides. This allows more sensitive discrimination between the variant sequence and the reference sequence, especially when the variant sequence comprises an insertion.

In some embodiments, in particular where the NOI is a microsatellite, the hybridisation sequence of the reporter oligonucleotide preferably comprises a sequence which consists of repeats identical or complementary to the repeats of the NOI, and may advantageously also comprise a terminal sequence, such as one or two terminal sequences, herein termed helper sequence(s), which hybridise(s) to a strand of the first and second amplicons just upstream or just downstream to the repeats when the hybridisation sequence is hybridised thereto. In other words, the hybridisation sequence of the reporter oligonucleotide preferably comprises repeats, and additionally comprises 1 to 20 nucleotides, such as 1, 2, 3, 4, 5, 6, 7, 8, 9, 10, 11, 12, 13, 14, 15, 16, 17, 18, 19 or 20 nucleotides, which hybridise to the NOI or its complementary strand just upstream or downstream of the repetitive sequence.

The helper sequence may be 3′-terminal or 5′-terminal. Preferably, the reporter oligonucleotide comprises a 5′-terminal helper sequence which hybridises to a strand of the first and second amplicons just upstream or just downstream of the repeats when the hybridisation sequence is hybridised thereto. The helper sequence facilitates discrimination between the melting profiles, as shown in example 13. A helper sequence is termed strong if it has a Tm, when hybridised to the first or second amplicon, which is high. The skilled person knows well that hybridisation between two sequences is stronger if the sequences comprise a higher proportion of G/C compared to two hybridised sequences comprising a higher proportion of A/T; hence, a helper sequence comprising a higher proportion of G/C than A/T will be stronger than a helper sequence comprising a lower proportion of G/C than A/T. It is also known that longer sequences give a stronger hybridisation than shorter sequences, hence the strength of a helper sequence may be adjusted by increasing the length that hybridises to the first and/or second amplicons.

In some embodiments, the reporter oligonucleotide is useful for detecting a variant sequence indicating microsatellite instability, i.e. the reference sequence comprises a microsatellite sequence. When the reference sequence comprises a microsatellite sequence of M tandem repeats having a total length of n nucleotides, the length of the hybridisation sequence H of the reporter oligonucleotide is n″, wherein n″≥n+1, preferably n″≥n+1 or n″≥n+2.

The above reporter oligonucleotides may comprise a second quencher. Preferably, the second quencher is in a non-terminal region, i.e. located in the internal region of the reporter oligonucleotide. In other words, the second quencher is not located at the 5′-end or at the 3′-end or within 4 nucleotides from the 5′- or 3′-end.

Useful fluorophores and quenchers are known in the art and are readily available to the skilled person, who will have no difficulty selecting suitable fluorophores and quenchers.

Herein is also provided the use of such reporter oligonucleotides comprising hydrophobic nucleotides in methods for detecting a variant nucleic acid, in particular their use in the methods described herein. The reporter oligonucleotides may also be used in methods for predicting efficacy of treatment of a clinical condition, and in methods for predicting the presence of a clinical condition, in particular as described herein below.

Predicting Efficacy of Treatment of a Clinical Condition

The present disclosure also relates to methods for predicting the efficacy of treatment of a clinical condition in an individual in need thereof with a predetermined drug, wherein the efficacy of treatment of said clinical condition with said drug is associated with the presence of a variant sequence.

Thus, some mutations may be indicative of whether or not a certain drug may be efficient for treatment of an individual. In particular, specific mutations may be indicative of a specific response to a predetermined drug treatment. For example, the mutation may be indicative of whether an individual will respond positively to said drug treatment, whether the disease of an individual is resistant toward the given drug or whether an individual cannot tolerate a specific drug treatment.

Methods for predicting the efficacy of treatment of a clinical condition in an individual may comprise the steps of
a. providing a sample from said individual
b. performing the method for detecting the presence of a variant sequence as described herein to determine whether the sample comprises the variant sequence
wherein the presence of said variant sequence is indicative of whether said drug is efficient in treating said clinical condition in said individual.

The clinical condition may for example be cancer, or any of the clinical conditions, diseases or disorders described herein. Many mutations have been identified, which are indicative of whether a given cancer drug or combination of drugs is effective in treating a particular cancer.

In some embodiments, the variant sequence is a variant of a microsatellite, as described in detail herein above.

Predicting the Presence of a Clinical Condition

The present methods are also useful for predicting or even diagnosing the presence of any clinical condition associated with a particular mutation in an individual.

Such methods may comprise the steps of
a) Providing a sample from said individual
b) detecting the presence of a mutation associated with the clinical condition in the sample by performing the methods for detecting a variant sequence as described herein;
   wherein the presence of said variant sequence is indicative of said individual suffering from said clinical condition.

Numerous clinical conditions are known to be associated with particular mutations, and reporter oligonucleotides and sets of primers for detecting any such mutation may be designed. The present methods are particularly useful for detecting conditions associated with microsatellite instability, when used to detect a variant sequence of a microsatellite, preferably wherein the corresponding wild type or reference sequence is at least 15 nucleotides in length, as detailed above.

In one embodiment, the clinical condition is cancer, such as hereditary non-polyposis colorectal cancer. In another embodiment, the clinical condition is Lynch's syndrome.

The method may further comprise a step of treating said clinical condition. If the variant sequence indicating the presence of the clinical condition is found, the individual is classified as suffering from said clinical condition, and the method may further comprise the step of administering a therapeutic agent in an effective amount to said individual.

### EXAMPLES

### Example 1: Microsatellite Instability does not Necessarily Lead to Changes in Melting Temperature

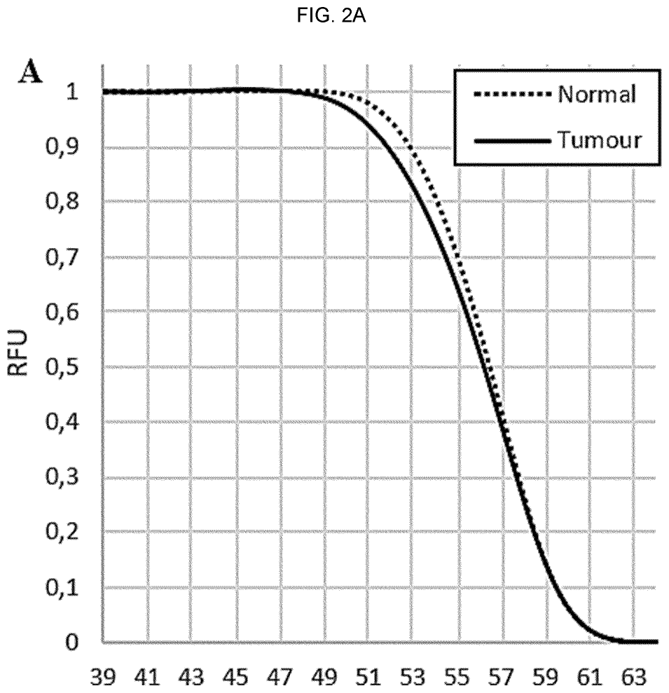

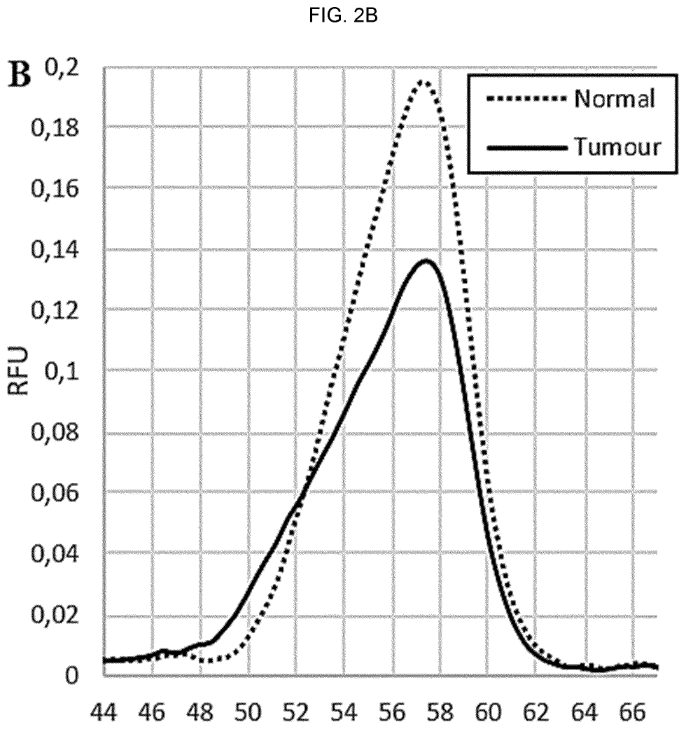

BAT25 assay using asymmetric PCR with primers hybridizing upstream and downstream of the region comprising the mononucleotide repeats. The assay is for detecting a variant sequence in the BAT25 microsatellite. The reporter oligonucleotide used in this example carried a FAM fluorophore at the 5′-end and a BHQ-1 quencher at the 3′-end, as well as an internal quencher. The reporter oligonucleotide hybridises to positions 142 to 171 of SEQ ID NO: 1, and has an overhang in its 3′-end and a helper sequence in its 5′-end, which hybridises upstream of the mononucleotide repeats. In addition, it comprises a hydrophobic nucleotide within 10 nucleotides from the 5′-end and a hydrophobic nucleotide within 10 nucleotides from the 3′-end. 1-2 ng/μL normal tissue DNA and tumour tissue DNA from CRC or endometrial samples from a microsatellite instable patient were tested. Initial normalization interval was 39-40° C., and the final normalization interval was 63-64° C. Two melt curves were obtained (FIG. 2A), which were transformed to negative first derivative curves (FIG. 2B). The two curves were not identical (maximal difference between the amplitudes of the two curves was ˜0.07 RFU (absolute value), not shown) when applying bilinear normalization and adjusting the temperature by applying a temperature shift intensity threshold of 0.1 RFU, which is above the 0.05 RFU threshold. A difference of 0.07 is above the 0.05 RFU threshold set for this particular assay and hence the sample is classified as unstable for BAT25. The negative first derivative curves are shown in FIG. 2B. As can be seen, the normal tissue had a Tₘ of 57.31° C. and the tumour tissue a Tₘ of 57.41° C. Such a small difference is usually not significant and could be due to different salt concentrations in the samples, or instrument variance.

From this example it can be concluded that the melting temperature is not necessarily changed significantly from a normal to a mutated tumour sample, but the shape of the melt curve is changed, which is even more easily visualised when applying bilinear normalisation and temperature shift. The normal and the tumor samples can thus be discriminated using the shape difference between the HRM curves, even with very low differences in Tₘ.

### Example 2: Bilinear Normalisation

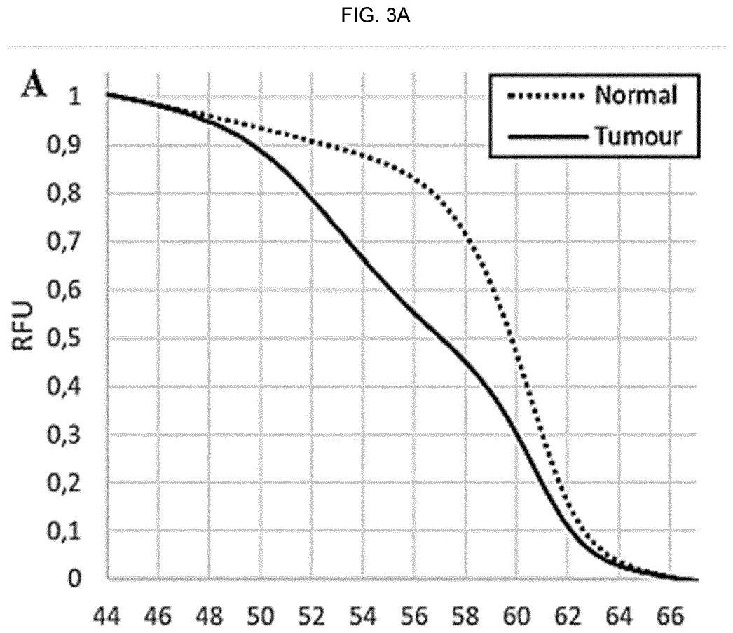

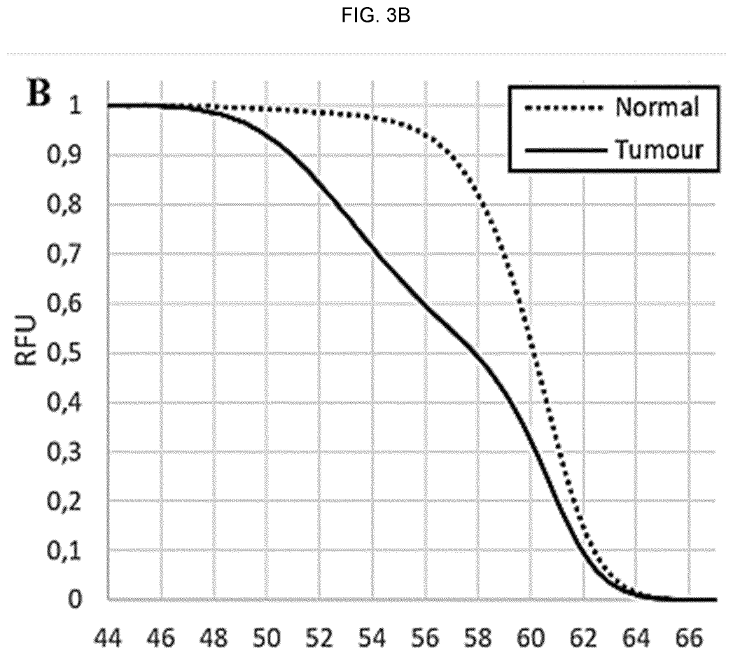

NR22 assay using asymmetric PCR with primers hybridizing upstream and downstream of the region comprising the mononucleotide repeats. The assay is for detecting a variant sequence in the NR22 microsatellite. The reporter oligonucleotide used in this example carried a FAM fluorophore at the 5′-end and a BHQ-1 quencher at the 3′-end, as well as an internal BHQ-1 quencher. The reporter oligonucleotide hybridises to positions 143 to 172 of SEQ ID NO: 4 and has an overhang (2 nucleotides) in its 3′-end and a helper sequence (9 nucleotides; leading to an increase in Tm of 14.2° C.) in its 5′-end, which hybridises upstream of the mononucleotide repeats. In addition, it comprises a hydrophobic nucleotide within 10 nucleotides from the 5′-end and a hydrophobic nucleotide within 10 nucleotides from the 3′-end. 1-2 ng/μL FFPE purified normal and tumour tissue DNA from a microsatellite instable patient was tested. Two melt curves were obtained (FIG. 3). FIG. 3A shows the melt curve without applying bilinear normalisation or temperature shift. FIG. 3B shows the melt curve after applying bilinear normalisation (no temperature shift).

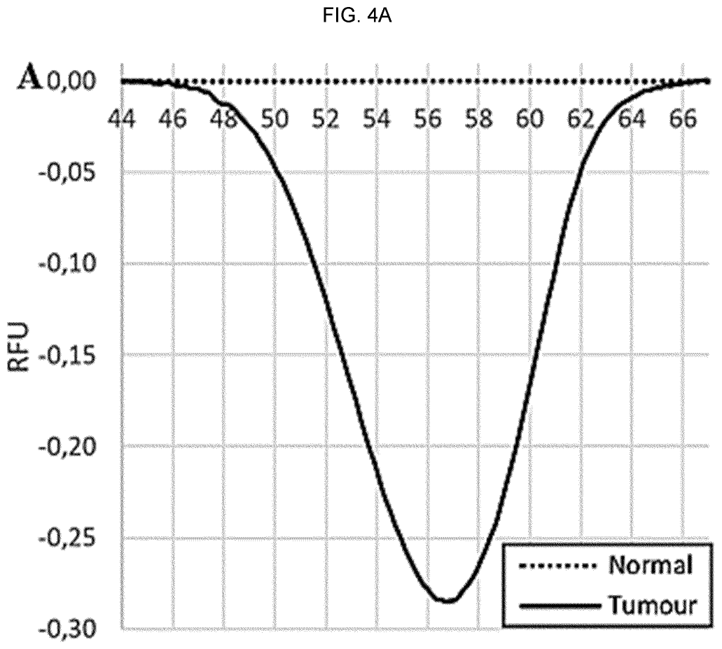

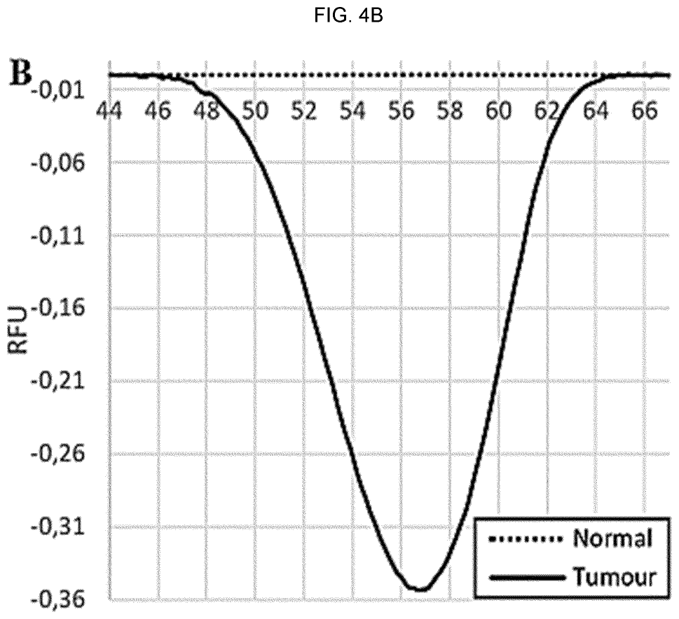

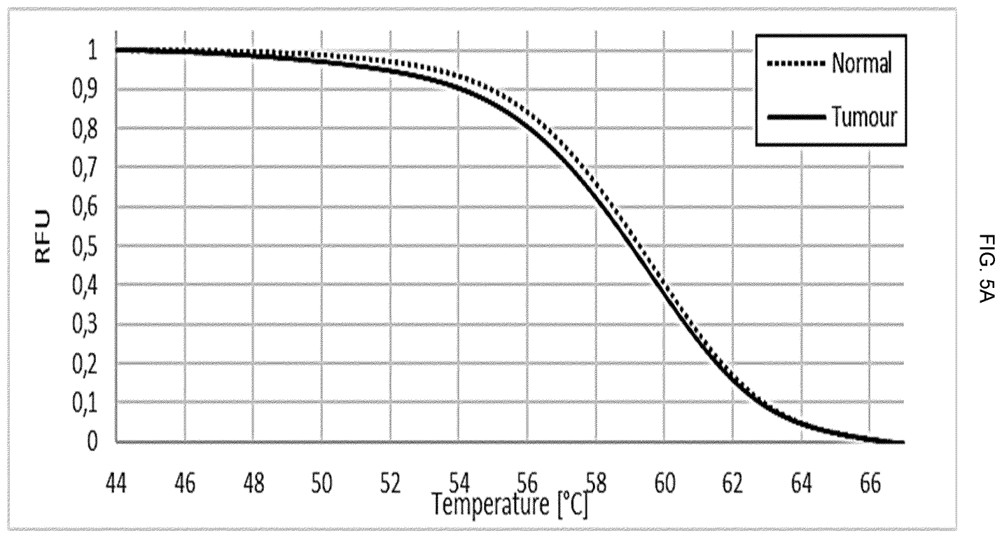

FIG. 4 shows the difference, D_{T}, in fluorescence between the reference (fluorescence set to be zero at any given temperature; the sample is from healthy cells of the patient) and the tumour sample as a function of temperature. This is also called a difference plot, a difference curve or a difference graph. The maximum difference, maxD_{T}, is measured as the absolute value of the maximum amplitude of the difference curve. Data shown after applying standard normalisation (FIG. 4A) or after applying bilinear normalisation (no temperature shift) (FIG. 4B).

This example shows that bilinear normalisation can compensate for the different decreases in fluorescence seen before and after the melt of different samples, and helps discriminate HRM profiles for a normal (healthy) sample and a tumor sample.

### Example 3: Bilinear Normalization and Temperature Shift can Reduce the Difference Between Melt Curves

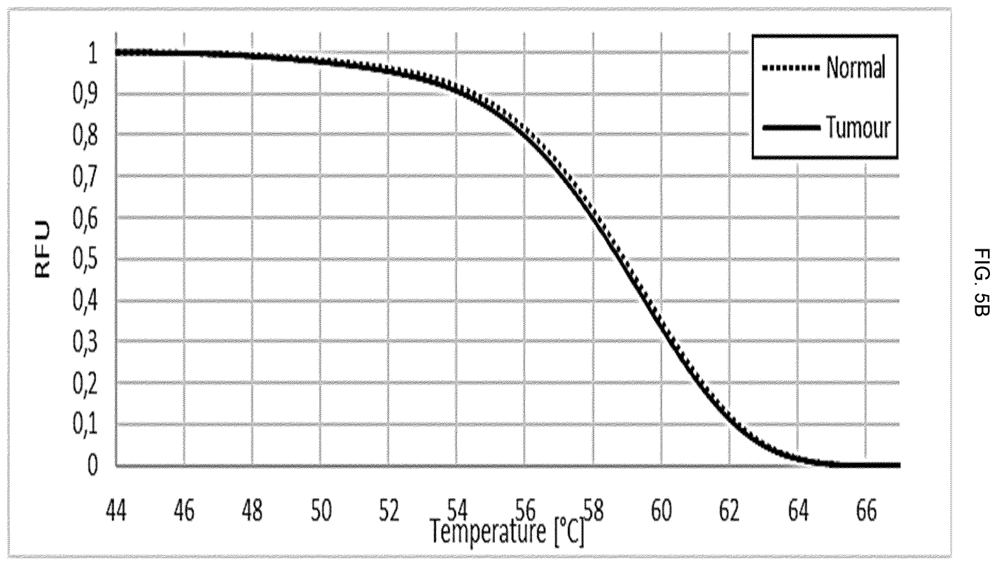

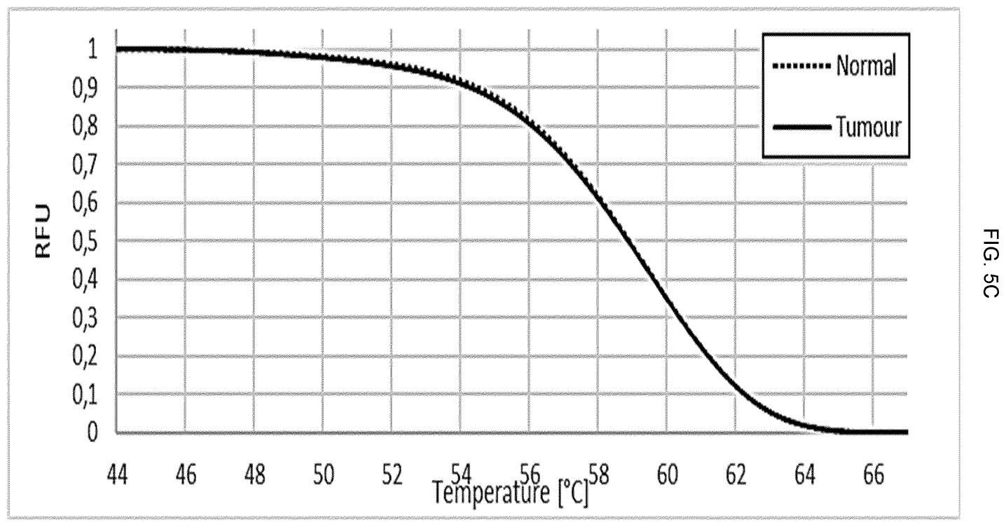

NR24 assay using asymmetric PCR. The assay is for detecting a variant sequence in the NR24 microsatellite. The reporter oligonucleotide used in this example carried a FAM fluorophore at the 5′-end and a BHQ-1 quencher at the 3′-end, as well as an internal quencher. The reporter oligonucleotide hybridises to positions 158 to 187 of SEQ ID NO: 5, and has an overhang in its 3′-end and a helper sequence (leading to an increase in Tm of 14.8° C.) in its 5′-end, which hybridises upstream of the mononucleotide repeats. In addition, it comprises a hydrophobic nucleotide within 10 nucleotides from the 5′-end and a hydrophobic nucleotide within 10 nucleotides from the 3′-end. 1-2 ng/μL normal tissue DNA and tumour tissue DNA from the same microsatellite stable patient were tested. Standard normalisation with initial interval 44-45° C. (set to value 1) and final interval 66-67° C. (set to value 0) was applied. Results are shown in FIG. 5.

When bilinear normalization and temperature shift intensity threshold were not applied, the difference between the curves was ˜0.04 RFU (FIG. 5A). When applying bilinear normalization but no temperature shift intensity threshold, the difference between the curves was ˜0.02 RFU (FIG. 5B). When bilinear normalization and temperature shift intensity threshold of 0.1 RFU were applied, the difference between the curves was ˜0.01 RFU (FIG. 5C).

From this example it can be concluded that the bilinear normalization and temperature shift can reduce the difference between the melting curves of healthy and normal samples from microsatellite stable patients. This reduces the risk of false positives.

### Example 4: Temperature Shift Intensity Threshold can Neutralize the Change in Melting Temperature Caused by Different Salt Concentration in DNA Buffers

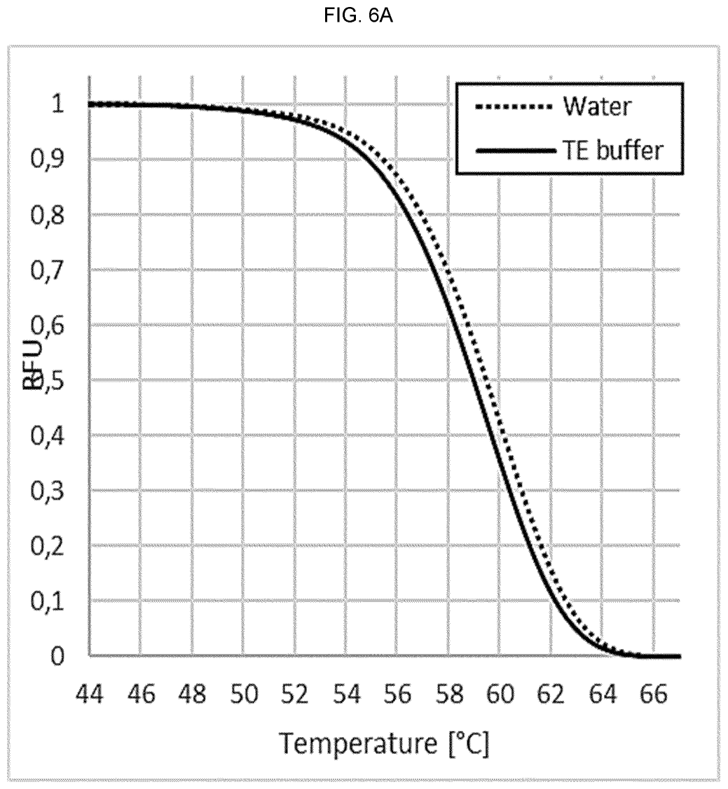

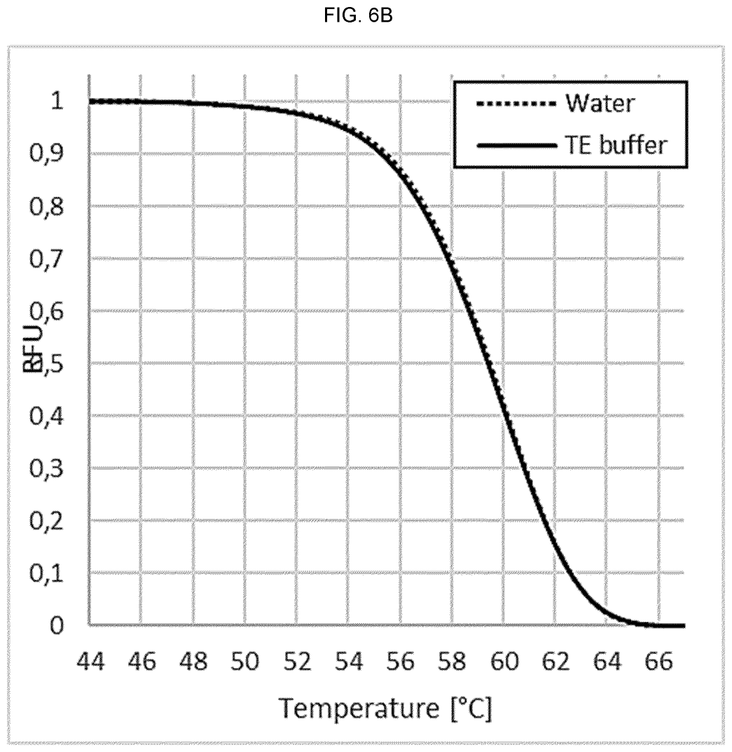

NR24 assay using asymmetric PCR. The assay is for detecting a variant sequence in the NR24 microsatellite. The reporter oligonucleotide used in this example carried a FAM fluorophore at the 5′-end and a BHQ-1 quencher at the 3′-end, as well as an internal BHQ-1 quencher. The reporter oligonucleotide hybridises to positions 158 to 187 of SEQ ID NO: 5, and has an overhang in its 3′-end (2 nucleotides) and a helper sequence in its 5′-end (leading to an increase in Tm of 14.8° C.), which hybridises upstream of the mononucleotide repeats. In addition, it comprises a hydrophobic nucleotide within 10 nucleotides from the 5′-end and a hydrophobic nucleotide within 10 nucleotides from the 3′-end. Normal tissue DNA was diluted from 40 ng/μL to 2 ng/μL in water and TE buffer respectively. Initial normalization was done in the area 44-45° C., and the final normalization was done in the area 66-67° C. Results are shown in FIG. 6.

When applying bilinear normalization but no temperature shift, the maximum difference, maxD_{T}, between the curves was ˜0.07 RFU (FIG. 6A). When bilinear normalization and temperature shift with an intensity threshold of 0.1 RFU were applied, maxD_{T} was ˜0.01 RFU (FIG. 6B).

From this example it can be concluded that applying temperature shift can reduce the differences between two samples, when that difference is caused by different salt concentration in the DNA buffers. Thus, after application of bilinear normalisation and temperature shift, aliquots of one sample can yield identical HRM profiles even if the aliquots were obtained with different buffers. This greatly reduces the risk of false positive callings, as the assay is less vulnerable for variations of salt and buffer concentrations between the samples compared.

### Example 5: Temperature Shift Creates a Possibility to Use One Universal Reference Sample

MONO27 assay using asymmetric PCR with primers hybridizing upstream and downstream of the region comprising the mononucleotide repeats. The assay is for detecting a variant sequence in the MON27 microsatellite. The reporter oligonucleotide used in this example carried a FAM fluorophore at the 5′-end and a BHQ-1 quencher at the 3′-end, as well as an internal BHQ-1 quencher. The reporter oligonucleotide hybridises to positions 300 to 333 of SEQ ID NO:6, and has an overhang in its 3′-end and a helper sequence (leading to an increase in Tm of 10.8° C.) in its 5′-end, which hybridises upstream of the mononucleotide repeats. In addition, it comprises a hydrophobic nucleotide within 10 nucleotides from the 5′-end and a hydrophobic nucleotide within 10 nucleotides from the 3′-end. 1-2 ng/μL FFPE purified normal tissue DNA from 16 different patients were tested. Initial normalization interval was 44-45° C., and the final normalization interval was 66-67° C.

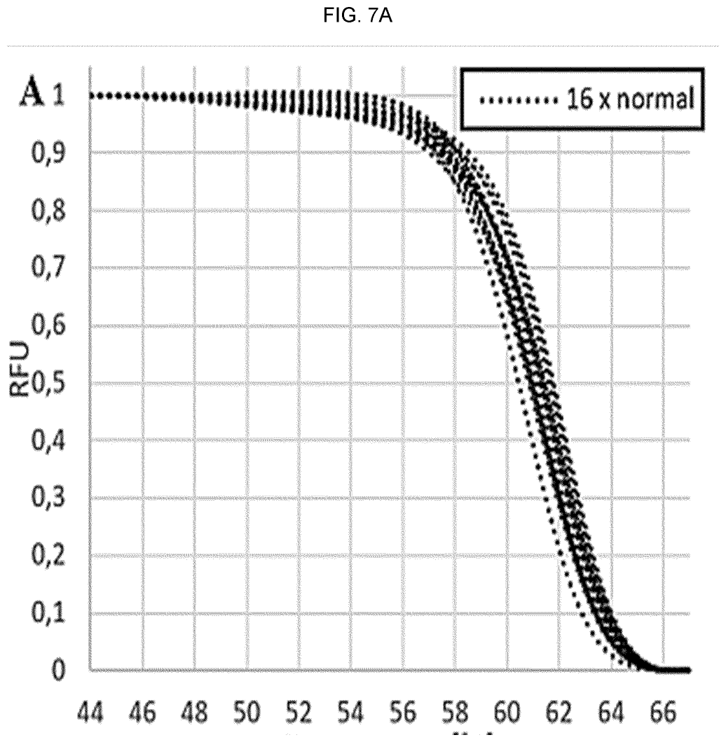

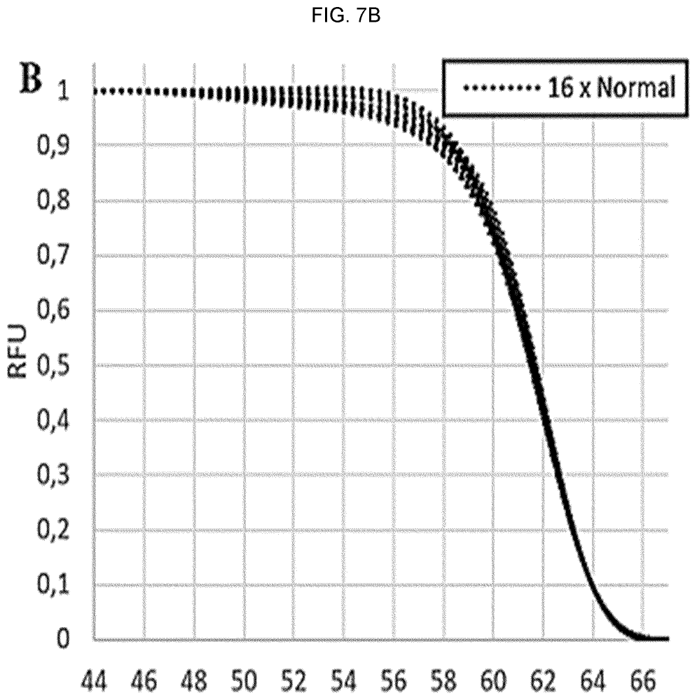

FIG. 7A shows HRM curves, where bilinear normalization was applied but temperature shift was not applied. The maximum difference, maxD_{T}, between the curves was ˜0.25 RFU. FIG. 7B shows HRM curves, where bilinear normalization and temperature shift with an intensity threshold of 0.1 RFU were applied, the maxD_{T}, was reduced to ˜0.06 RFU.

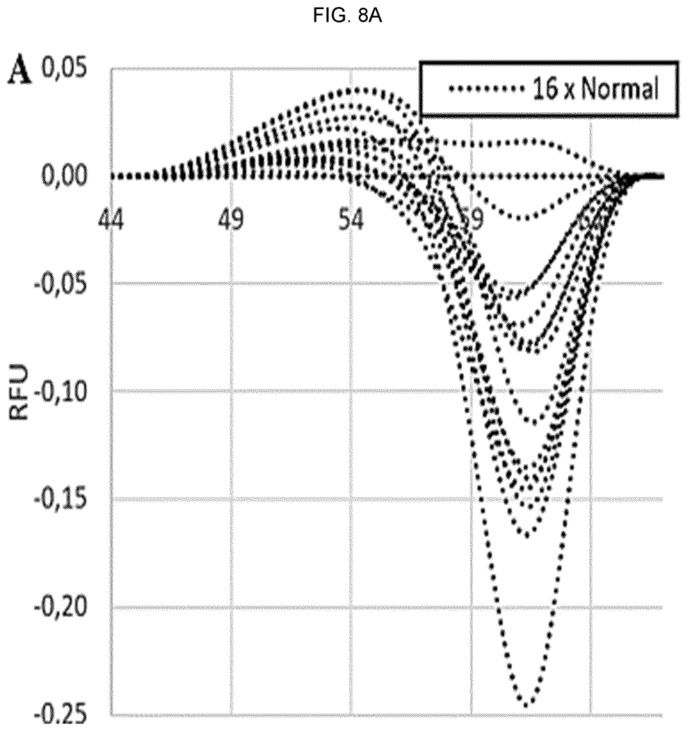

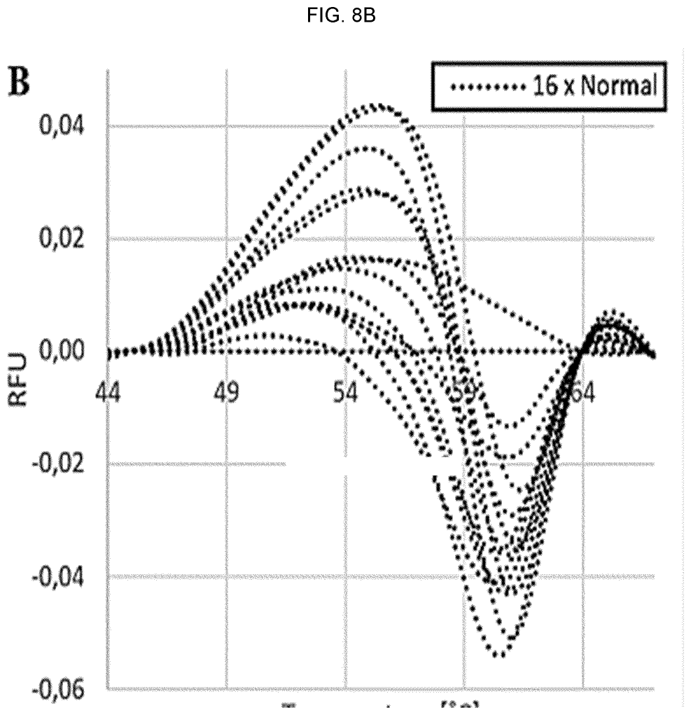

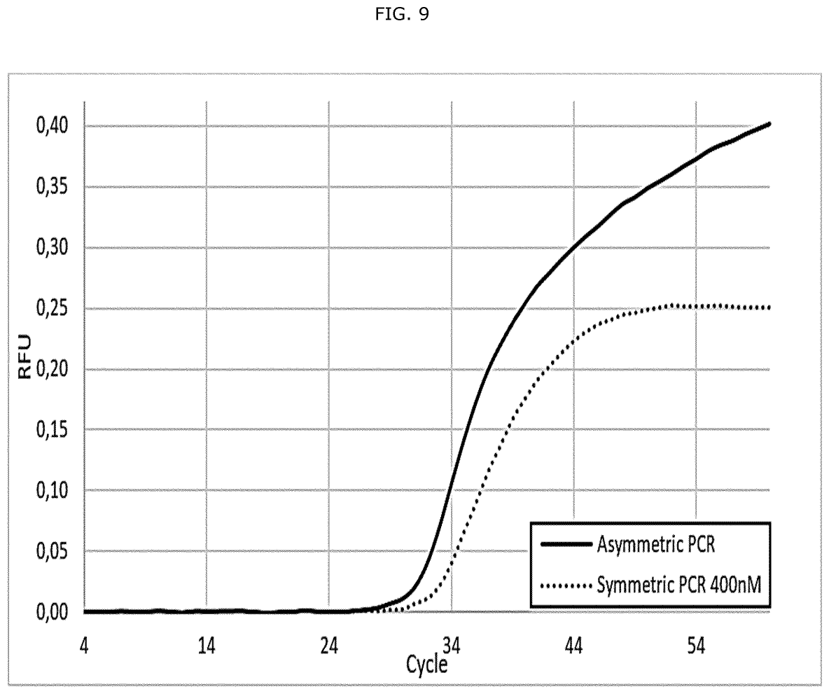

FIG. 8A and FIG. 8B show the difference plots of the HRM curves of FIG. 7A and FIG. 7B, respectively.

From this experiment it can be concluded that applying temperature shift reduces the difference between healthy samples from different patients. This creates the possibility to use one universal reference sample, instead of paired normal and tumour samples for each patient.

### Example 6: Asymmetric PCR Creates More Single Stranded Amplicon

NR22 assay using asymmetric PCR with primers hybridizing upstream and downstream of the region comprising the mononucleotide repeats. The assay is for detecting a variant sequence in the NR22 microsatellite. The reporter oligonucleotide used in this example carried a FAM fluorophore at the 5′-end and a BHQ-1 quencher at the 3′-end, as well as an internal BHQ-1 quencher. The reporter oligonucleotide hybridises to positions 142 to 172 of SEQ ID NO: 4 and has an overhang in its 3′-end and a helper sequence (leading to an increase in Tm of 14.2° C.) in its 5′-end, which hybridises upstream of the mononucleotide repeats. In addition, it comprises a hydrophobic nucleotide within 10 nucleotides from the 5′-end and a hydrophobic nucleotide within 10 nucleotides from the 3′-end.

1-2 ng/μL blood purified normal tissue DNA was tested. On the figure, the real-time PCR curves are seen. The symmetric PCR curve stops increasing in RFU after cycle 45, and for asymmetric PCR the amplification continues after the exponential phase with linear amplification.

From this experiment it can be seen that asymmetric PCR creates more of the single DNA strand comprising the sequence to which the hybridization sequence of the reporter oligonucleotide can bind.

### Example 7: Asymmetric PCR Creates Higher Signal to Noise and Sharper Melt Curves

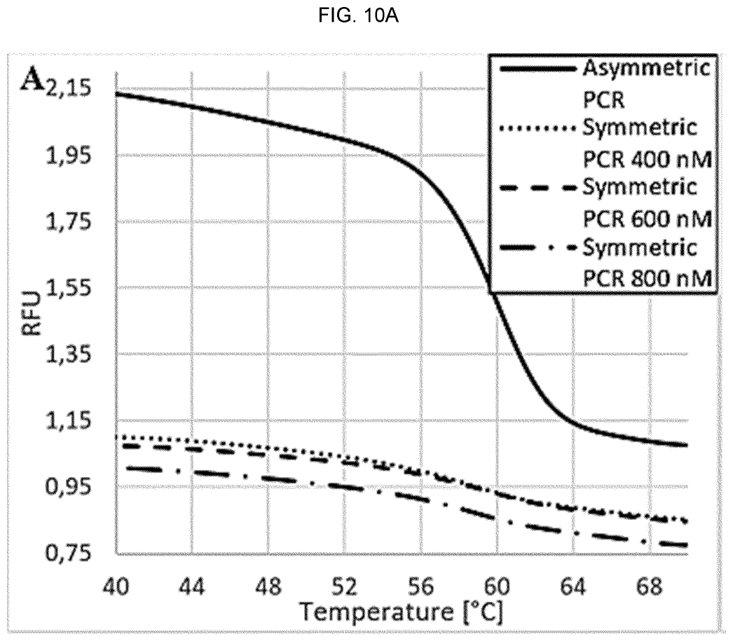

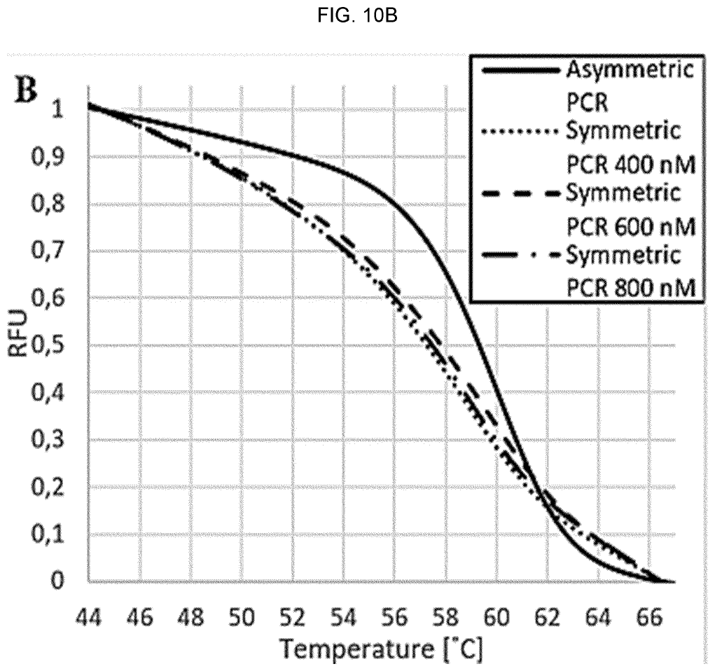

The assay was done as described in example 6. The normalised HRM curves were obtained and are shown in FIG. 10.

From this experiment it can be concluded that asymmetric PCR creates higher signal to noise ratio and creates a “sharper” melt profile.

### Example 8: Asymmetric PCR Makes it Easier to Discriminate Between MSS and MSI Patients

NR22 assay was performed as described in example 6. 1-2 ng/μL FFPE purified normal and tumour tissue DNA from a microsatellite unstable patient was tested.

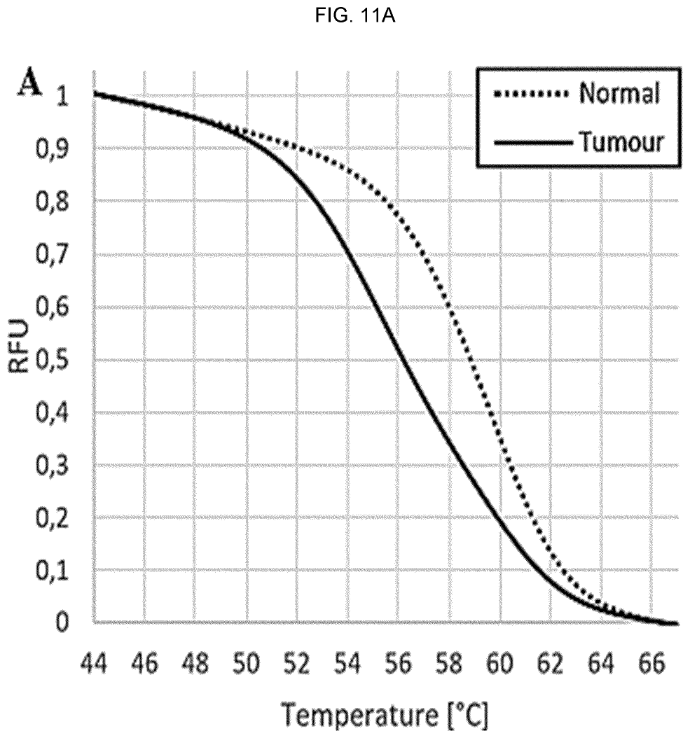

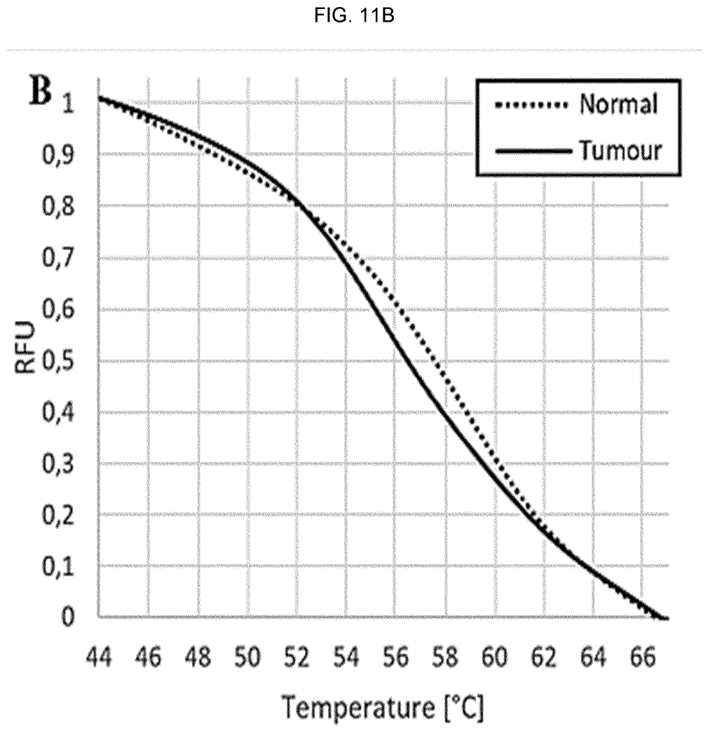

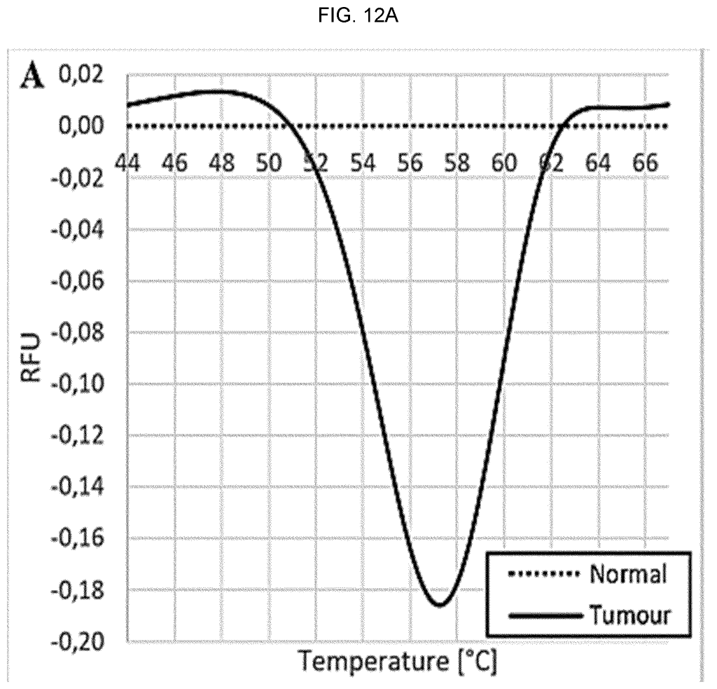

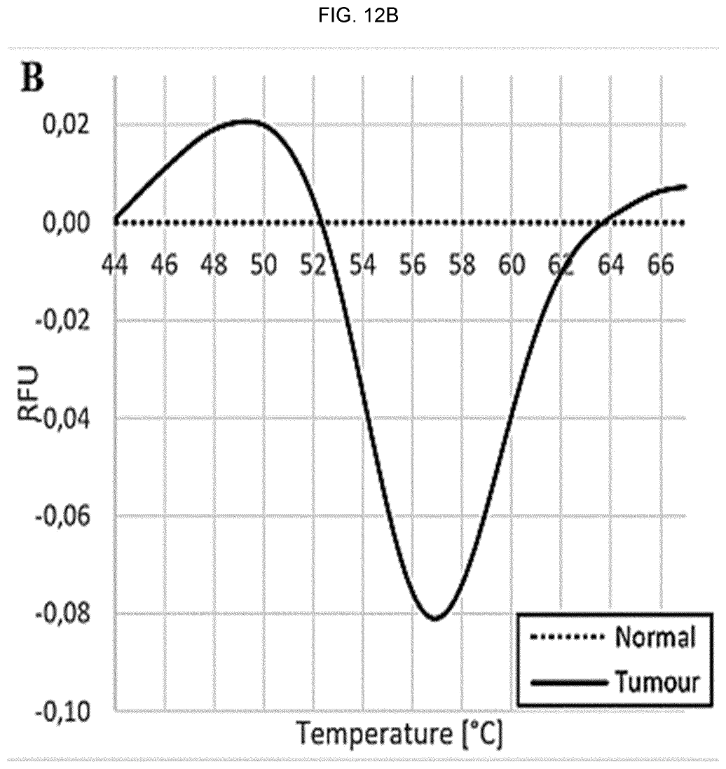

FIG. 11 shows the HRM curves obtained after applying standard normalisation with asymmetric PCR (FIG. 11A) or with symmetric PCR (FIG. 11B). The corresponding difference curves are shown in FIG. 12A and FIG. 12B, respectively.

From this example it can be concluded that amplification with asymmetric PCR creates larger difference between the normal and tumour tissue curve for a microsatellite unstable patient compared to symmetric PCR. Thus asymmetric PCR makes it easier to discriminate between MSS and MSI patients.

### Example 9: Single-End Overhang in the Reporter Oligonucleotide can Increase Melting Temperature

NR22 assay using asymmetric PCR with primers hybridizing upstream and downstream of the region comprising the mononucleotide repeats. The assay is for detecting a variant sequence in the NR22 microsatellite.

Two different reporter oligonucleotides were used in this experiment. The first reporter oligonucleotide used in this example carried a FAM fluorophore at the 5′-end and a BHQ-1 quencher at the 3′-end. The first reporter oligonucleotide hybridises to positions 148 to 177 of SEQ ID NO: 4. The hybridisation sequence also comprises a 4-nucleotide long helper sequence (leading to an increase in Tm of 7.5° C.) at the 5′-end and a 5-nucleotide long helper sequence (leading to an increase in Tm of 3.6° C.) at the 3′-end, which are complementary to the regions of NR22 just upstream and downstream of the repeats. The second reporter oligonucleotide carried a FAM fluorophore at the 5′-end and a BHQ-1 quencher at the 3′-end as well as an internal quencher. The second reporter oligonucleotide hybridises to positions 143 to 172 of SEQ ID NO: 4 and has an overhang in its 3′-end and a 9-nucleotide long helper sequence only in its 5′-end (leading to an increase in Tm of 14.2° C.), which hybridises upstream of the mononucleotide repeats. In addition, both reporter oligonucleotides comprise a hydrophobic nucleotide within 10 nucleotides from the 5′-end and a hydrophobic nucleotide within 10 nucleotides from the 3′-end. 1-2 ng/μL blood purified normal tissue DNA was tested.

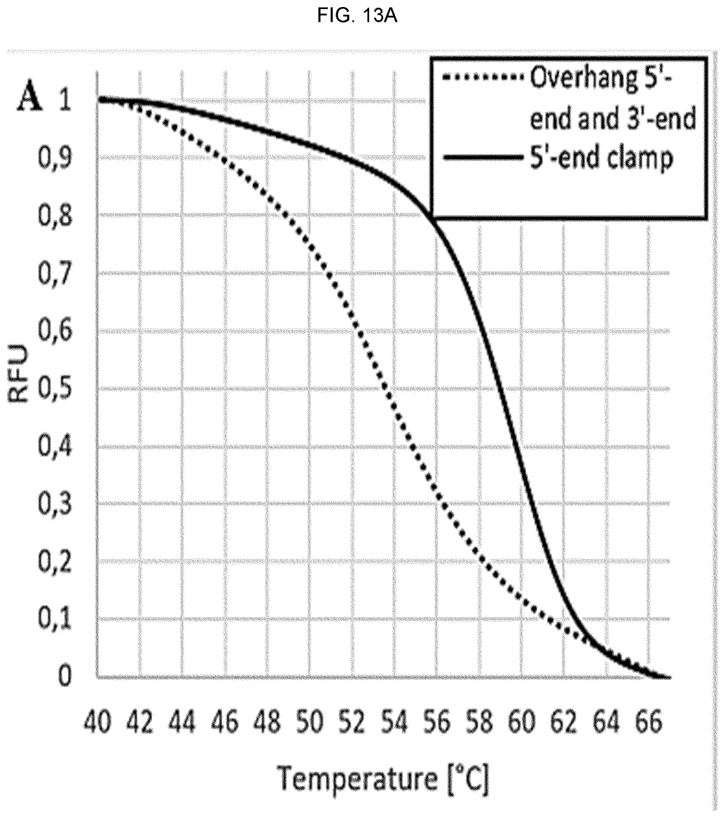

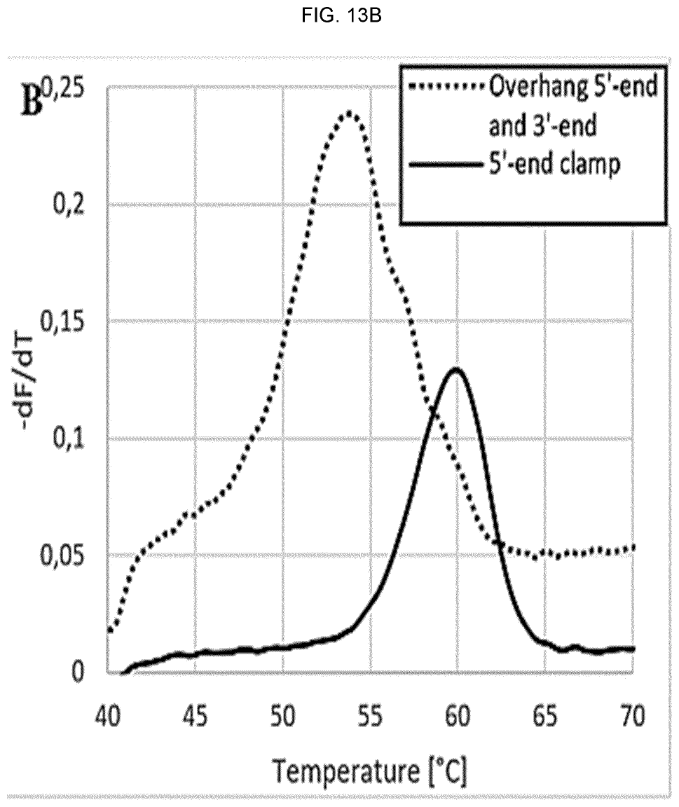

Results for the assay using the first reporter oligonucleotide are shown as dashed lines in FIG. 13; results using the second reporter oligonucleotide are shown as full lines. The melt curves are shown in FIG. 13A and the derivative curves in FIG. 13B. Bilinear normalization and temperature shift were not applied.

It is seen that the melting temperature for the second reporter oligonucleotide is higher than for the first reporter oligonucleotide. The lower background fluorescence for the second reporter oligonucleotide is likely due to the presence of the additional, internal quencher.

From this experiment it can be seen that having one longer helper sequence in one end instead of two smaller helper sequences in each end can increase the melting temperature of the probe, and can be an advantage when investigating mononucleotide repetitive microsatellites.

### Example 10: Double-Quenching the Reporter Oligonucleotide Increases the Signal to Noise Ratio

BAT26 assay using asymmetric PCR with primers hybridizing upstream and downstream of the region comprising the mononucleotide repeats. The assay is for detecting a variant sequence in the BAT26 microsatellite. 1-2 ng/μL blood purified normal tissue DNA was tested. A: HRM curves, with standard normalization applied. Two reporter oligonucleotides were used in this example, both carrying a FAM fluorophore at the 5′-end and a BHQ-1 quencher at the 3′-end. As an example of a reporter oligonucleotide that was only single quenched, not DQ probe, BAT26 reporter oligonucleotide FAM 5′-CZCCTTTTTTTTTTTTTTTTTTTTTTTTTTTTAZC-3′ BHQ-1 (SEQ ID NO: 7) was used. BAT26 reporter oligonucleotide FAM 5′-CZCCTTTTTTTTTTXTTTTTTTTTTTTTTTTTTAZC-3′ BHQ-1 (SEQ ID NO: 8) was used to illustrate a double quenched, DQ, reporter oligonucleotide; X indicates the internal quencher.

The reporter oligonucleotides hybridise to positions 220 to 251 of SEQ ID NO: 2 and has an overhang in its 3′-end and a helper sequence in both its 5′-end (leading to an increase in Tm of 5.2° C.) and its 3′-end (leading to an increase in Tm of 1.9° C.), which hybridise upstream and downstream of the mononucleotide repeats. In addition, it comprises a hydrophobic nucleotide within 10 nucleotides from the 5′-end and a hydrophobic nucleotide within 10 nucleotides from the 3′-end.

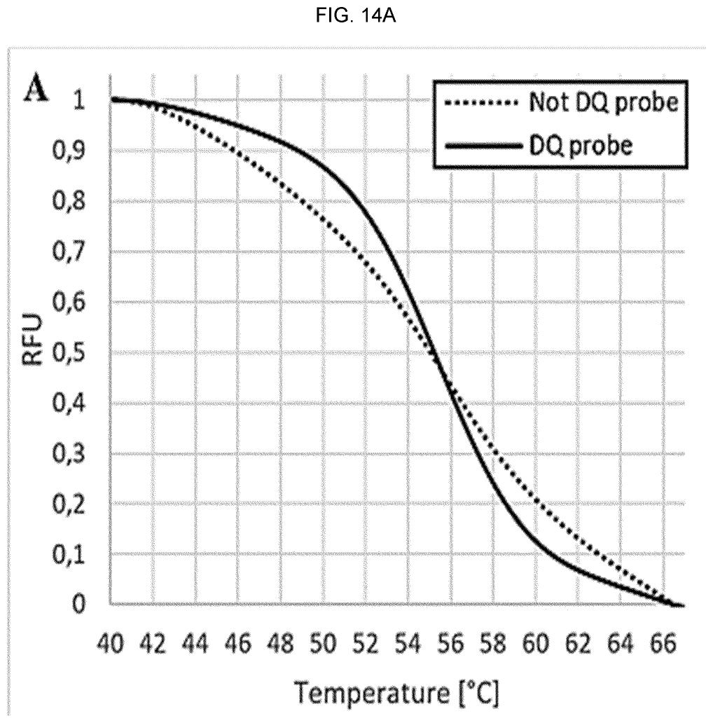

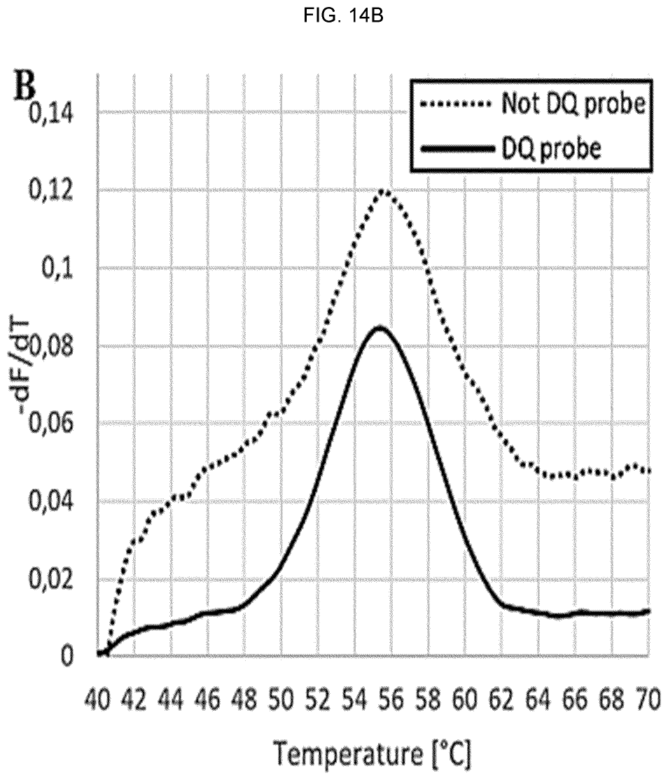

HRM curves and negative first derivative curves are shown in FIGS. 14A and B, respectively. It is seen that the background fluorescence of the single quenched reporter oligonucleotide (not DQ probe) is higher compared to the double-quenched reporter oligonucleotide (DQ probe). From this experiment it can be concluded that DQ reporter oligonucleotides reduce the fluorescence noise and make the melt sharper compared to single-quenched reporter oligonucleotides. The use of double-quenched reporter oligonucleotides can thus facilitate discrimination between normal and tumor samples.

### Example 11: Additional Nucleotide Repeats in the Hybridisation Sequence of the Reporter Oligonucleotide Makes it Possible to Detect Longer Microsatellites

NR21 assay was performed using asymmetric PCR. The assay is for detecting a variant sequence in the NR21 microsatellite. The reporter oligonucleotide used in this example carried a FAM fluorophore at the 5′-end and a BHQ-1 quencher at the 3′-end, as well as an internal quencher. The reporter oligonucleotide hybridises to positions 189 to 214 of SEQ ID NO: 2, and has an overhang in its 3′-end and a helper sequence in its 5′-end (leading to an increase in Tm of 15.2° C.), which hybridises downstream of the mononucleotide repeats. In addition, the reporter oligonucleotide comprises 2 hydrophobic nucleotides within 5 nucleotides from the 5′-end and one hydrophobic nucleotide within 5 nucleotides from the 3′-end. The hybridisation sequence of the reporter oligonucleotide comprises 22 T nucleotide repeats; the NR21 microsatellite consists of 21 repeats in most normal cells. The reporter oligonucleotide thus has one additional T compared to the reference sequence.

The reporter oligonucleotide was tested against artificial targets of 21, 22 or 23 adenine repeats.

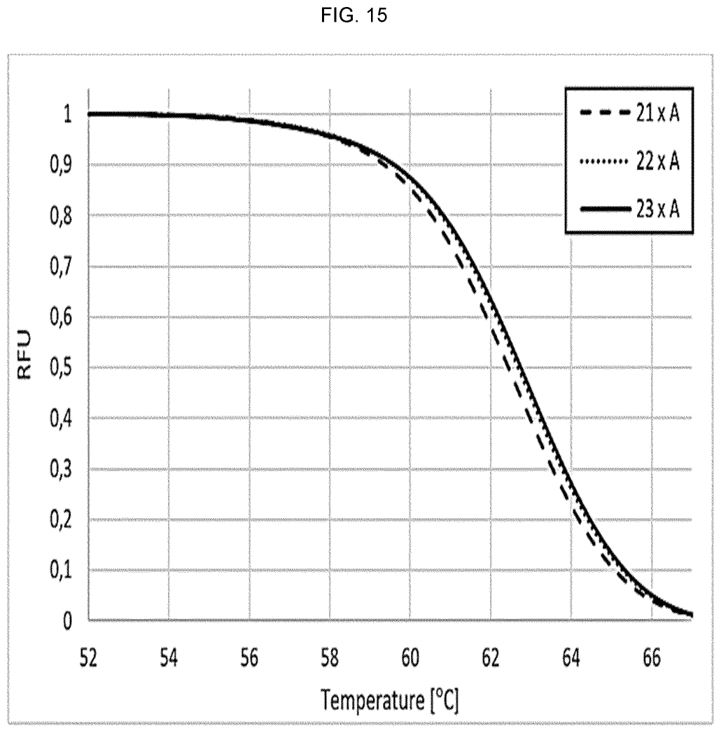

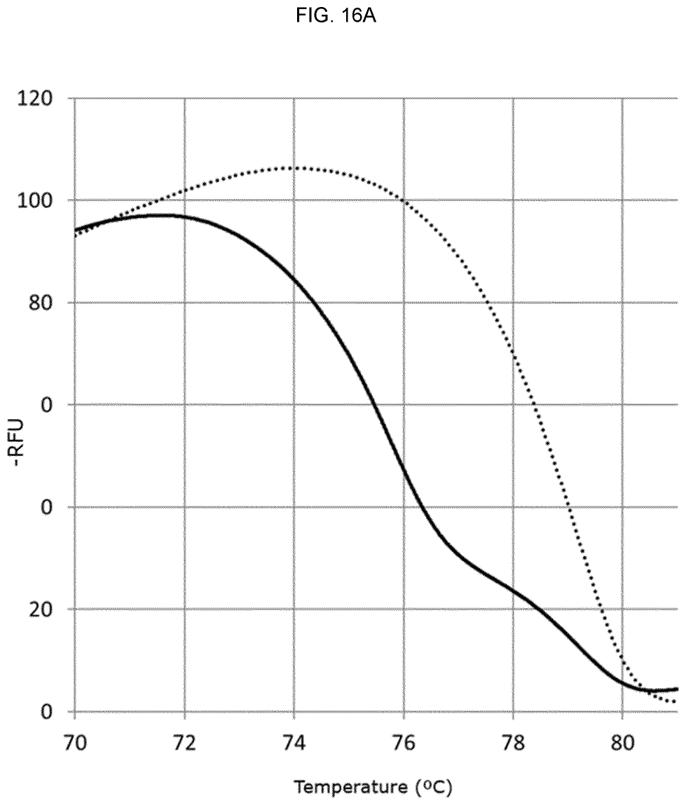

HRM curves are shown in FIG. 15 after applying bilinear normalisation but no temperature shift. From this experiment it can be concluded that adding one extra repeat in the hybridisation sequence of the reporter oligonucleotide allows detection of microsatellites, which are longer than the 21 repeats of the reference.

### Example 12: A Single Point Mutation Changes Melting Temperature with Several Degrees

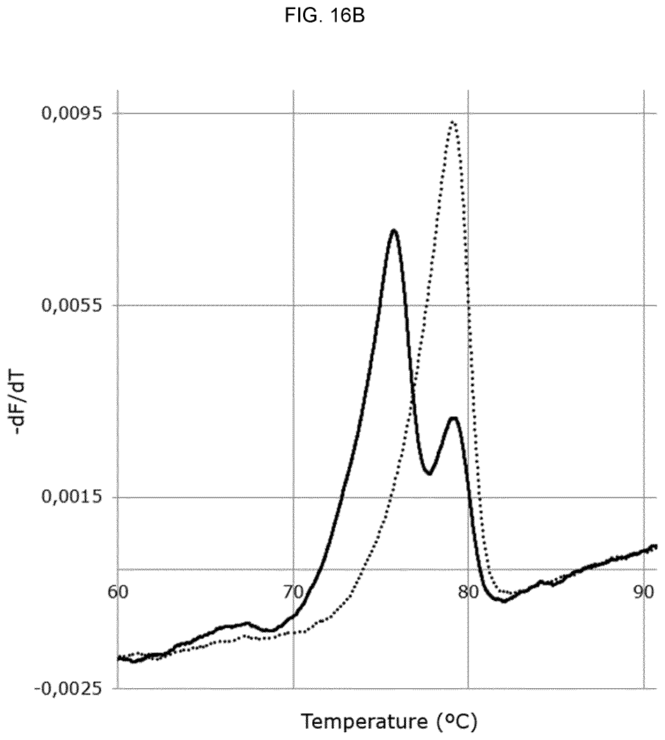

KIT Exon 13 assay to detect variant sequence in KIT exon 13. The PCR was carried out as asymmetric PCR. The reporter oligonucleotide carried a FAM fluorophore in its 5′-end and a BHQ1 quencher in its 3′-end. 1 ng/ul FFPE purified tissue DNA from a wild-type sample and FFPE purified tumor tissue from a patient sample were examined. Initial normalization interval was 70-71° C. and final normalization interval was 80-81° C. Results are shown in FIG. 16. Wild-type tissue gives a Tₘ of 79.1° C. and tumor tissue gives two Tₘ's; 75.7° C. and 79.1° C.

From this experiment it can be concluded that melting temperature of the probe is altered with up to 3.4° C. and that shape of the melt curve is significantly changed in case of a single point mutation. Healthy and tumor samples can thus be discriminated.

### Example 13: Stronger Single-End Helper Sequence Increases Difference and Facilitates Discrimination Between Mutant and Wild-Type

NR24 assay using asymmetric PCR with primers hybridizing upstream and downstream of the region comprising the mononucleotide repeats. The assay is for detecting a variant sequence in the NR24 microsatellite.

Two different reporter oligonucleotides were used in this experiment. The first reporter oligonucleotide carries a FAM fluorophore at the 5′-end and a BHQ-1 quencher at the 3′-end as well as an internal BHQ-1 quencher. The first reporter oligonucleotide hybridizes to positions 159 to 187 of SEQ ID NO: 5, and has an overhang in its 3′-end (2 nucleotides) and a 5-nucleotide long helper sequence (leading to an increase in Tm of 10.5° C.) in its 5′-end, which hybridizes upstream of the mononucleotide repeats. In addition, it comprises two hydrophobic nucleotides within 10 nucleotides from the 5′-end and a hydrophobic nucleotide within 10 nucleotides from the 3′-end. The second reporter is described in example 4. 1-2 ng/μL FFPE purified normal and tumour tissue DNA from a microsatellite unstable patient was tested.

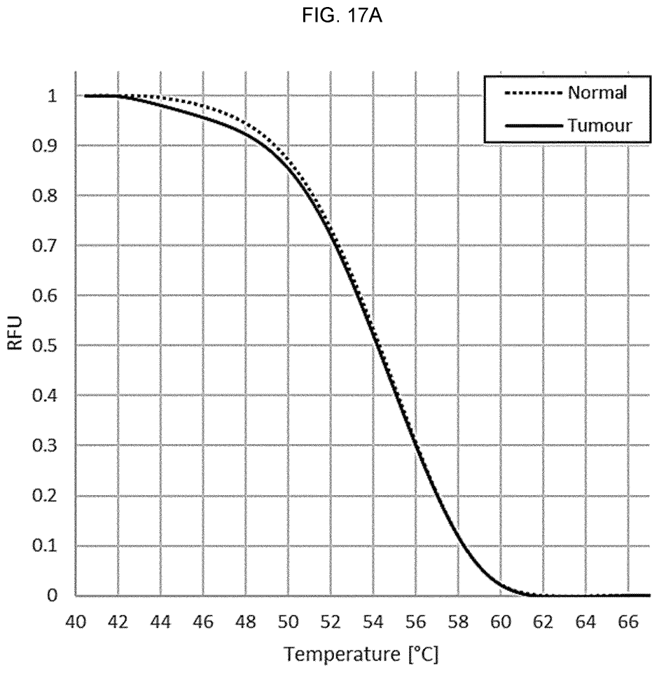

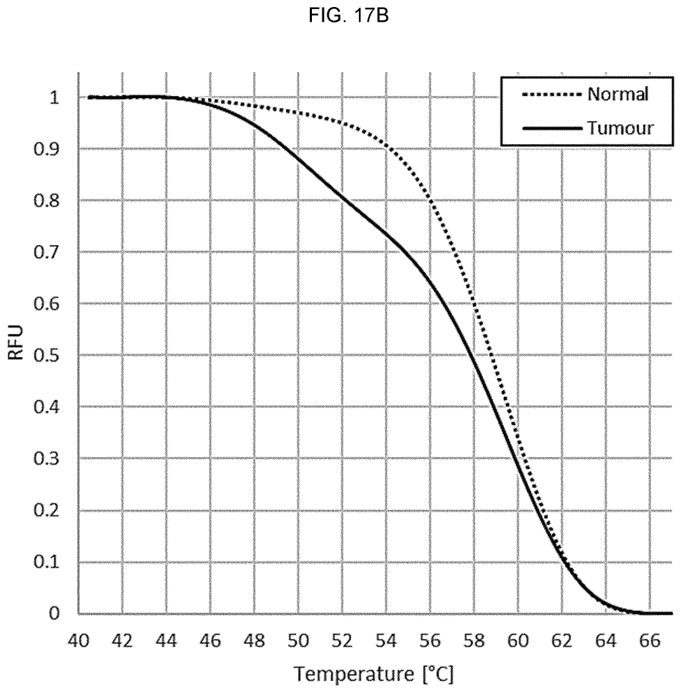

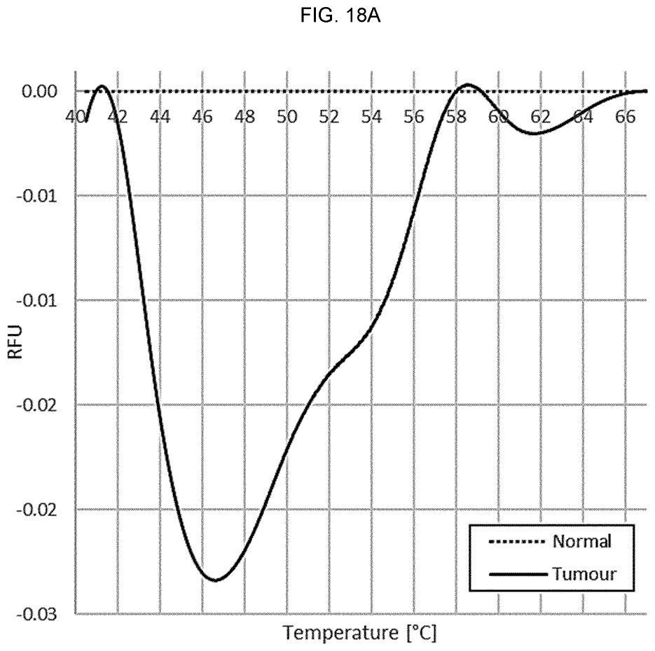

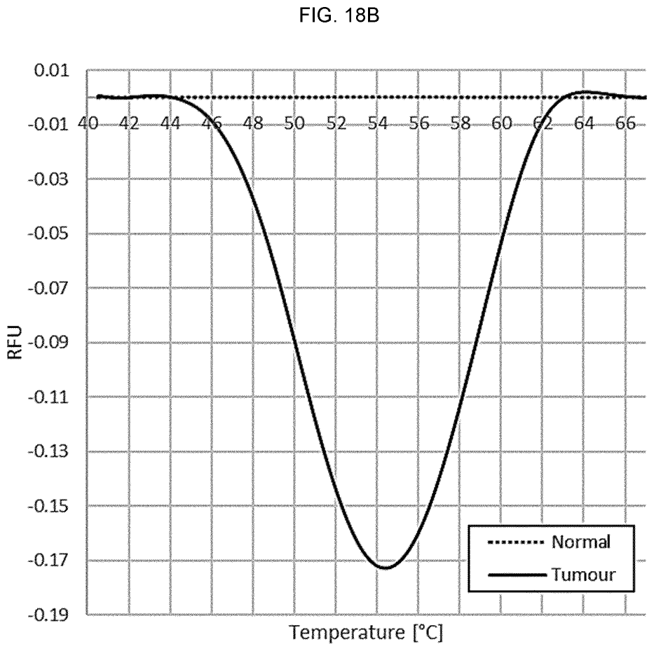

Initial normalization was done in the area 40.5-41.5 and ° C., and the final normalization was done in the area 66-67° C. Bilinear normalization and temperature shift at 0.05 RFU was applied. Results are shown in FIGS. 17 and 18; compare FIGS. 17A and 18A (results with the first reporter oligonucleotide) and FIGS. 17B and 18B (results with the second reporter oligonucleotide).

The second helper sequence when hybridised results in an increase in Tm higher (14.8° C., i.e. 4.3° C. higher) than the first helper sequence, which helps discriminating the two melt profiles.

It is seen that a stronger helper sequence increases the maxD_{T} of the mutant. The first reporter oligonucleotide with the weak helper sequence yielded a maxD_{T}˜0.03 RFU between normal and tumour tissue. This is below the threshold set for NR24 marker, and the mutant would therefore incorrectly be categorised as wild type (FIG. 18A). The second reporter oligonucleotide with stronger helper sequence yielded a maxD_{T}˜0.17, and thus the sample is correctly categorised as a mutant (FIG. 18B).

From this experiment, it can be concluded that a stronger helper sequence can increase the difference between wild-type and mutant, which can be an advantage when investigating mononucleotide repetitive microsatellites.

Sequences

| Sequence | | |
|---|---|---|
| ID NO: | Description | Sequence |
| 1 | BAT25 | GCCATCATGGAGGATGACGAG |
| | | TTGGCCCTAGACTTAGAAGAC |
| | | TTGCTGAGCTTTTCTTACCA |
| | | GGTGGCAAAGGGCATGGCTT |
| | | TCCTCGCCTCCAAGAATGTA |
| | | AGTGGGAGTGATTCTCTAAA |
| | | GAGTTTTGTGTTTTGTTTTT |
| | | TTGATTTTTTTTTTTTTTTT |
| | | TTTTTTTTTGAGAACAGAGC |
| | | ATTTTAGAGCCATAGTTAAA |
| | | ATGCAGAATGTCATTTTGAA |
| | | GTGTGGTAACCAAAAGCAGA |
| | | GGAAATTTAGTTTCTTCATG |
| | | TTCCAACTGCTGTCTCTTTG |
| | | GAATTCCTGTTCTAATTTA |
| 2 | BAT26 | GTTTTTTAAAATCTTTAGAA |
| | | CTGGATCCAGTGGTATAGAA |
| | | ATCTTCGATTTTTAAATTCT |
| | | TAATTTTAGGTTGCAGTTTC |
| | | ATCACTGTCTGCGGTAATCA |
| | | AGTTTTTAGAACTCTTATCA |
| | | GATGATTCCAACTTTGGACA |
| | | GTTTGAACTGACTACTTTTG |
| | | ACTTCAGCCAGTATATGAAA |
| | | TTGGATATTGCAGCAGTCAG |
| | | AGCCCTTAACCTTTTTCAGG |
| | | TAAAAAAAAAAAAAAAAAAA |
| | | AAAAAAAAGGGTTAAAAATG |
| | | TTGAATGGTTAAAAAATGTT |
| | | TTCATTGACATATACTGAAG |
| | | AAGCTTATAAAGGAGCTAAA |
| | | ATATTTTGAAATATTATTAT |
| | | ACTTGGATTAGATAACTAGC |
| | | TTTAAATGGCTGTATTTTTC |
| | | TCTCCCCTCCTCCACTCCAC |
| | | T |
| 3 | NR21 | CCCTTTCTAAATGCGTATTC |
| | | GTGTAAATATATTGGGAGAG |
| | | AGCTTTGAATTAGAACGTCC |
| | | TTTTCCGAAATAGGAACCAC |
| | | TGCTACTCTCTAAAAAAGGC |
| | | AAGCAGATAAAAGAGAACAC |
| | | GAAAAATATTCCTACTCCGC |
| | | ATTCACACTTTCTGGTCACT |
| | | CGCGTTTACAAACAAGAAAA |
| | | GTGTTGCTAAAAAAAAAAAA |
| | | AAAAAAAAAGGCCAGGGGAG |
| | | ACATACATTTAAATATAAAA |
| | | ATAGAACTGTGCCAGCGACT |
| | | CCGGCTGGAATTCTGCTGAA |
| | | AGGGATGTGTCTTCAGAAAC |
| | | C |
| 4 | NR22 | TCTCCAAAGTTGATCTGATT |
| | | GTAAATATTAAACTGACATC |
| | | TTTATGTTGCAGGTAAAGGA |
| | | CCTGGATAATCGAGGCTTGT |
| | | CAAGGACATAAATGTCACGT |
| | | CCAGCTCTGATATGCTTCGC |
| | | ACTGAGCACATCACATTTAG |
| | | GACGTTGAAGATTTTTTTTT |
| | | TTTTTTTTTTTTAATATGCA |
| | | GTTTGTAAGAACAAAACTGG |
| | | ATGGCATCAGAATTGTCTGG |
| | | AAGTTTTGTCTTGGGCAGTA |
| | | TGGGCTGGGCCAAATGAAAT |
| | | GATTTTTATAATTCTAAACA |
| | | GGTTACCAAAT |
| 5 | NR24 | CAAATGACCCCTTCCTGCCC |
| | | ATCACTGCCTTCCTCAAGAC |
| | | CTAAAATAGCTCCCTATTTA |
| | | GTGAAAAATTATCTGAATAT |
| | | TTAAGGTCTGCCTTAACGTG |
| | | ATCCCCATTGCTGAATTTTA |
| | | CCTCCTGACTCCAAAAACTC |
| | | TTCTCTTCCCTGGGCCCAGT |
| | | CCTATTTTTTTTTTTTTTTT |
| | | TTTTTTTGTGAGACAGAGTC |
| | | TCACTCTGTCACCCAGGTTG |
| | | GAATGCAATGGCACAATCTC |
| | | CGCTCACTGCAAGCTCCGCC |
| | | TCCCGGGTTCACGCCATTCT |
| | | CCTGCCTCAGCCTCCCGAAT |
| | | A |
| 6 | MONO27 | TTAAAAAGCAAAAAATTGGC |
| | | TGGGCACAGTGGCTCACACC |
| | | TGTAATTCCAGCACTTCAGG |
| | | AGGCTGAGGCAGGAAGATTG |
| | | CTTGAGCCCAGAAATTCAAT |
| | | ACCAGCCTGGGCAAGATAAT |
| | | GAGACCCCATCTCTGCAAAA |
| | | AATGAAAAATAAACTAGCCA |
| | | GGTGTGGTGGCATGCACCTA |
| | | TGCCCCCAGCCACTCAAGAG |
| | | GCTGAGGTGGGAGGATCACT |
| | | TGAGCCCAGGAGGTCGAGGC |
| | | TGCAGTGAGCTGTGATTGCA |
| | | CTACACTCCAGCCTGGGTGA |
| | | CATAATGAGACCCTGTCTCA |
| | | AAAAAAAAAAAAAAAAAAAA |
| | | AAAAAAACTGGAGCCAGGCA |
| | | CAGTAGGACAGTAGTAATTC |
| | | ATGCCTGTAGTCCTAGCTTA |
| | | CATTTCAGTAGAATTGTTTA |
| | | GGGATATTAAGTCTGTTGCT |
| | | TAAACTTGTAAAACTTTATT |
| | | ATATATTGAAAAACATGCGT |
| | | TGACTAATTTTATGAGTATT |
| | | AATTGTCTTCTTTTACAGTA |
| | | ACTGGACCTCTGTCAGAACT |
| | | GCAAATTGCATATGTTAGCA |
| | | GAGAAACACTGCAGGTAAAT |
| | | CAAGTGCTAATTCAAAAATA |
| | | ACATTTTTCATTAAACTAAG |
| | | G |
| 7 | Reporter | CZCCTTTTTTTTTTTTTTTT |
| | oligonucleotide | TTTTTTTTTTTTAZC |
| 8 | Reporter | CZCCTTTTTTTTTTXTTTTT |
| | oligonucleotide | TTTTTTTTTTTTTAZC |

### REFERENCES

Boland et al. (1998) “A National Cancer Institute workshop on microsatellite instability for cancer detection and familial predisposition: development of international criteria for the determination of microsatellite instability in colorectal cancer,” Cancer Res 58:5248-5257

Rodriguez-Bigas et al. (1997) “A National Cancer Institute workshop on hereditary nonpolyposis colorectal cancer syndrome: meeting highlights and Bethesda guidelines,” J Natl Cancer Inst 89:1758-1762 Hunter and Sanders (1990) J. Am Chem. Soc. 112: 5525-5534

### WO 2017/045689

### WO 03/052132

### Items

1. A method for detecting the presence of a variant sequence in a target nucleic acid sequence consisting of two strands and comprising nucleotide(s) of interest (NOI), wherein said target nucleic acid sequence consists of a variant sequence or of a reference sequence, said method comprising the steps of:
   a) Providing a first sample comprising nucleic acids suspected of comprising said variant sequence;
   b) Providing a second sample comprising nucleic acids comprising said reference sequence, wherein the second sample is a reference sample;
   c) Providing a reporter oligonucleotide;
   d) Providing a set of primers consisting of a first primer and a second primer, wherein the set of primers together are capable of amplifying the target nucleic acid sequence;
   e) Amplifying the target nucleic acid sequence in the presence of said first sample, said first primer and said second primer, thereby obtaining a first amplicon comprising nucleic acids suspected of comprising a variant sequence; and amplifying the target nucleic acid sequence in the presence of said second sample, said first primer and said second primer, thereby obtaining a second amplicon comprising the reference sequence, wherein the second amplicon is a reference amplicon;
   f) Performing high-resolution melt (HRM) analysis of the first amplicon, thereby obtaining a first HRM profile characterised by a first melt curve, and performing HRM analysis of the second amplicon, thereby obtaining a second HRM profile, characterised by a second melt curve wherein the second HRM profile is a reference profile characterised by a reference melt curve; wherein each amplicon comprises a first strand and a second strand, wherein the HRM analysis involves hybridisation of the reporter oligonucleotide to one strand of each amplicon, detection of a signal emitted by the fluorophore, and obtaining the first and the second melt curves;
   wherein the reporter oligonucleotide is a sequence of in the range of 10 to 50 nucleotides into which in the range of 2 to 10 hydrophobic nucleotides have been inserted,
   wherein the reporter oligonucleotide comprises a first fluorophore, preferably in its 5′-end or within 4 nucleotides from the 5′-end, and a first quencher, preferably in its 3′-end or within 4 nucleotides from the 3′-end and
   wherein the reporter oligonucleotide comprises a hybridization sequence H, wherein
   at least one hydrophobic nucleotide is positioned at the 5′-end or within 10 nucleotides from the 5′-end of the reporter oligonucleotide; and/or
   at least one hydrophobic nucleotide is positioned at the 3′-end or within 10 nucleotides from the 3′-end of the reporter oligonucleotide; and
   wherein the hydrophobic nucleotide has the structure

X—Y-Q
wherein
   X is a nucleotide or nucleotide analogue or a backbone monomer unit capable of being incorporated into the backbone of a nucleic acid or nucleic acid analogue,
   Q is an intercalator which is not taking part in Watson-Crick hydrogen bonding; and
   Y is a linker moiety linking said nucleotide or nucleotide analogue or backbone monomer unit and said intercalator; and
   wherein the hybridisation sequence is identical to a consecutive stretch of the sequence of a first strand of the target nucleic acid sequence, and
   wherein the hybridisation sequence is complementary to a consecutive stretch of the sequence of the second strand of the target nucleic acid sequence; and
   g) Comparing the first HRM profile to the reference HRM profile, wherein a difference between the first HRM profile and the reference HRM profile indicates that the first sample contains a variant sequence.
2. The method according to item 1, wherein the NOI comprises repeats, and wherein the hybridisation sequence of the reporter oligonucleotide consists of a repetitive sequence and a helper sequence in its 5′-end and/or in its 3′-end, wherein said helper sequence does not comprise repeats, and can hybridise to the first or the second strand of the first and second amplicons when the hybridisation sequence is hybridized thereto, preferably wherein the reporter oligonucleotide consists of a repetitive sequence and only one helper sequence in its 5′-end or its 3′-end.
3. The method according to item 2, wherein the helper sequence comprises or consists of 1 to 20 nucleotides, such as 1, 2, 3, 4, 5, 6, 7, 8, 9, 10, 11, 12, 13, 14, 15, 16, 17, 18, 19 or 20 nucleotides, preferably wherein the helper sequence comprises at least one hydrophobic oligonucleotide as defined in item 1.
4. The method according to any one of the preceding items, wherein at least one hydrophobic nucleotide as defined in item 1, such as 1, 2, or 3 hydrophobic nucleotides, is inserted within 1 to 10 nucleotides from the 3′-end of the reporter oligonucleotide, such as within 1, 2, 3, 4, 5, 6, 7, 8, 9 or 10 nucleotides from the 3′-end.
5. The method according to any one of the preceding items, wherein at least one hydrophobic nucleotide as defined in item 1, such as 1, 2, or 3 hydrophobic nucleotides, is inserted within 1 to 10 nucleotides from the 5′-end of the reporter oligonucleotide, such as within 1, 2, 3, 4, 5, 6, 7, 8, 9 or 10 nucleotides from the 5′-end.
6. The method according to any one of the preceding items, wherein the plurality of target nucleic acid sequences consists of BAT25 and BAT26, preferably the plurality consists of BAT25 as set forth in SEQ ID NO:1 and BAT26 as set forth in SEQ ID NO: 2.
7. The method according to any one of the preceding items, wherein the target nucleic acid sequence is one or more nucleic acid sequences comprising one or more of BAT25, BAT26, NR21, NR22, NR24 and MONO27, preferably BAT25 as set forth in SEQ ID NO:1, BAT26 as set forth in SEQ ID NO: 2, NR21 as set forth in SEQ ID NO: 3, NR22 as set forth in SEQ ID NO: 4, NR24 as set forth in SEQ ID NO: 5 and MONO27 as set forth in SEQ ID NO: 6.
8. The method according to any one of the preceding items, wherein the target nucleic acids are a plurality of target nucleic acid sequences consisting of BAT25, BAT26, NR21, NR22 and NR24, preferably the plurality consists of BAT25 as set forth in SEQ ID NO:1, BAT26 as set forth in SEQ ID NO: 2, NR21 as set forth in SEQ ID NO: 3, NR22 as set forth in SEQ ID NO: 4, and NR24 as set forth in SEQ ID NO: 5.
9. The method according to any one of the preceding items, wherein the target nucleic acids are a plurality of target nucleic acid sequences consisting of BAT25, BAT26, NR22, NR24 and MONO27, preferably the plurality consists of BAT25 as set forth in SEQ ID NO:1, BAT26 as set forth in SEQ ID NO: 2, NR22 as set forth in SEQ ID NO: 4, NR24 as set forth in SEQ ID NO: 5 and MONO27 as set forth in SEQ ID NO: 6.
10. The method according to any one of the preceding items, wherein the target nucleic acids are a plurality of target nucleic acid sequences consisting of BAT25, BAT26, NR21, NR22, NR24 and MONO27, preferably the plurality of target nucleic acid sequences consists of BAT25 as set forth in SEQ ID NO:1, BAT26 as set forth in SEQ ID NO: 2, NR21 as set forth in SEQ ID NO: 3, NR22 as set forth in SEQ ID NO: 4, NR24 as set forth in SEQ ID NO: 5 and MONO27 as set forth in SEQ ID NO: 6.
11. The method according to any one of the preceding items, wherein amplifying in step e) is performed by polymerase chain reaction (PCR), preferably by asymmetric PCR wherein the first and second primers are provided in different amounts, thereby directing the PCR towards amplifying more of the second strand of each amplicon than of the first strand of each amplicon.
12. The method according to any one of the preceding items, wherein the first sample has been isolated from an individual suffering from or suspected of suffering from a disease, such as a cancer, preferably the cancer is hereditary non-polyposis colorectal cancer.
13. The method according to any one of the preceding items, wherein the NOI is a microsatellite.
14. The method according to any one of the preceding items, wherein the reference sequence comprises a microsatellite sequence of M tandem repeats having a total length of n nucleotides, and wherein the variant sequence has M′ tandem repeats having a total length of n′ nucleotides, wherein M and M′ are different integers.
15. The method according to any one of the preceding items, wherein the length of the hybridisation sequence H of the reporter oligonucleotide is n″, wherein n″≥n+1, preferably n″≥n+1 or n″≥n+2.
16. The method according to any one of the preceding items, wherein the reporter oligonucleotide comprises a second quencher positioned at a non-terminal position of the reporter oligonucleotide.
17. The method according to any one of the preceding items, wherein the first sample is a sample of a tissue comprising or suspected of comprising cells with mutations characteristic of said disease.
18. The method according to any one of the preceding items, wherein the reference sample is isolated from the same individual as the first sample, optionally from a non-diseased tissue, or wherein the reference sample is isolated from a healthy individual.
19. The method according to any one of the preceding items, wherein step f) comprises a step of transforming the first and the second HRM melt curves to obtain a negative first derivative of the first and second melt curves, wherein a difference between the first HRM profile and the reference HRM profile is a difference between the negative first derivatives of the first and second melt curves.
20. The method according to any one of the preceding items, wherein the fluorescence of the first melt curve and the fluorescence of the second melt curve are normalized and wherein the difference between the HRM profiles is a difference between the fluorescence of said first and second melt curves as a function of temperature.
21. The method according to any one of the preceding items, wherein the difference between the first HRM profile and the reference HRM profile is measured as an absolute maximal difference in relative fluorescence units within the boundaries of upper and lower normalisation areas between the first and the second HRM curves.
22. The method according to any one of the preceding items, wherein the HRM analysis comprises a step of bilinear normalisation of the first and second melt curves.
23. The method according to any one of the preceding items, wherein the HRM analysis comprises a step of applying a temperature adjustment at a relative fluorescent unit (RFU) to the first and second melt curves and/or to the negative first derivatives of the first and second melt curves.
24. The method according to any one of the preceding items, wherein the second primer comprises a sequence of at least 15 nucleotides, which is complementary to a consecutive sequence of the target nucleic acid sequence.
25. The method according to any one of the preceding items, wherein the variant sequence comprises or consists of insertion of one or more nucleotides compared to the reference sequence.
26. The method according to any one of the preceding items, wherein the variant sequence comprises or consists of deletion of one or more nucleotides compared to the reference sequence.
27. The method according to any one of the preceding items, wherein the reporter oligonucleotide has the following general structure

5′-(N)ₐ—Z—(N)_{d}Z—(N)ₑ—Z—(N)_{b}-3′
wherein
N is any nucleotide or nucleotide analogue; and
Z is a hydrophobic nucleotide as defined in item 1; and
the total number of nucleotides or nucleotide analogues is at least 10; and
a and b individually are integers in the range of 0 to 4; and
d and e individually are integers in the range of 1 to 19; and
a+b+d+e at least 10; and
(N)ₐ—(N)_{d}—(N)ₑ—(N)_{b} is identical to the reference sequence.
28. The method according to any one of the preceding items, wherein the reporter oligonucleotide has the following general structure:

5′-(N)ₐ—Z—(N)_{f}Z—(N)_{g}—Z—(N)ₕ—Z—(N)_{b}-3′
wherein
N is any nucleotide or nucleotide analogue; and
Z is a hydrophobic nucleotide as defined in item 1; and
a and b individually are integers in the range of 0 to 4; and
f, g and h individually are integers in the range of 1 to 18; and
a+b+f+g+h is at least 10 and at the most 50; and
(N)ₐ—(N)_{f}—(N)_{g}—(N)ₕ—(N)_{b} is identical to a stretch of the target nucleic acid sequence comprising the reference sequence; or
   has the following general structure

5′-(N)ₐ—Z—(N)ᵢ—Z—(N)ⱼ—Z—(N)ₖ—Z—(N)ₗ—Z—(N)_{b}-3′
wherein
N is any nucleotide or nucleotide analogue; and
Z is a hydrophobic nucleotide as defined in item 1; and
a and b individually are integers in the range from 0 to 4; and
j, k and l individually are integers in the range from 1 to 17; and
a+b+i+j+k+l is at least 10 and at the most 50; and
(N)ₐ—(N)ᵢ—(N)ⱼ—(N)ₖ—(N)ᵢ—(N)_{b} is identical to a stretch of the target nucleic acid sequence comprising the reference sequence; or
   has the following general structure

5′-(N)ₐ—Z—(N)ₘ—Z—(N)ₙ—Z—(N)ₒ—Z—(N)ₚ—Z—(N)_{q}—Z—(N)_{b}-3′
wherein
   N is any nucleotide or nucleotide analogue; and
   Z is a hydrophobic nucleotide as defined in item 1; and
   a and b individually are integers in the range of 0 to 4; and
   m, n, o, p and q individually are integers in the range of 1 to 16; and
   a+b+m+n+o+p+q is at least 10 and at the most 50; and
   (N)ₐ—(N)ₘ—(N)ₙ—(N)ₒ—(N)ₚ—Z—(N)_{q}—(N)_{b} is identical to a stretch of the target nucleic acid sequence comprising the reference sequence.
29. The method according to any one of the preceding items, wherein at least one intercalator, Q, is selected from the group consisting of polyaromates and heteropolyaromates optionally substituted with one or more selected from the group consisting of hydroxyl, bromo, fluoro, chloro, iodo, mercapto, thio, cyano, alkylthio, heterocycle, aryl, heteroaryl, carboxyl, carboalkoyl, alkyl, alkenyl, alkynyl, nitro, amino, alkoxyl and amido.
30. The method according to any one of the preceding items, wherein the intercalator is selected from the group consisting of benzene, pentalene, indene, naphthalene, azulene, as-indacene, s-indacene, biphenylene, acenaphthylene, phenalene, heptalene, phenanthrane, fluoranthene, phenanthroline, phenazine, phenanthridine, anthraquinone, pyrene, anthracene, napthene, phenanthrene, flurene, picene, chrysene, naphtacene, acridones, benzanthracenes, stilbenes, oxalo-pyridocarbazoles, azidobenzenes, porphyrins and psoralens and derivatives thereof.
31. The method according to any one of the preceding items, wherein at least one, for example all backbone monomer unit(s) X is a phosphoramidite.
32. The method according to any one of the preceding items, wherein at least one linker, Y, comprises a chain of x atoms selected from the group consisting of C, O, S, N and P, optionally wherein said chain is substituted with one or more selected from the group consisting of C, H, O, S, N and P.
33. The method according to any one of the preceding items, wherein the target nucleic acid sequence is a plurality of target nucleic acid sequences.
34. A kit of parts for detecting the presence of a variant sequence in a target nucleic acid sequence consisting of two strands and comprising nucleotide(s) of interest (NOI), wherein said target nucleic acid sequence consists of a variant sequence or of a reference sequence, said kit of parts comprising:
   a) a reporter oligonucleotide comprising a first fluorophore, preferably in its 5′-end or within 4 nucleotides from the 5′-end, and a first quencher, preferably in its 3′-end or within 4 nucleotides from the 3′-end, wherein the reporter oligonucleotide is a sequence of in the range of 10 to 50 nucleotides into which in the range of 2 to 10 hydrophobic nucleotides have been inserted and wherein the reporter oligonucleotide comprises a hybridization sequence H,
   wherein
   at least one hydrophobic nucleotide is positioned at the 5′-end or within 10 nucleotides from the 5′-end of the reporter oligonucleotide; and/or
   at least one hydrophobic nucleotide is positioned at the 3′-end or within 10 nucleotides from the 3′-end of the reporter oligonucleotide; and
   wherein the hydrophobic nucleotide has the structure

X—Y-Q
wherein
   X is a nucleotide or nucleotide analogue or a backbone monomer unit capable of being incorporated into the backbone of a nucleic acid or nucleic acid analogue,
   Q is an intercalator which is not taking part in Watson-Crick hydrogen bonding; and
   Y is a linker moiety linking said nucleotide or nucleotide analogue or backbone monomer unit and said intercalator; and
   wherein the hybridisation sequence is identical to a consecutive stretch of the sequence of the first strand of the target nucleic acid, and wherein the hybridisation sequence is complementary to a consecutive stretch of the sequence of the second strand of the target nucleic acid; and
   b) a set of primers consisting of a first primer and a second primer, wherein the set of primers together are capable of amplifying the target nucleic acid.
35. The kit according to item 34, wherein the reporter oligonucleotide and the first primer and the second primer are as defined in any one of the preceding items.
36. A reporter oligonucleotide which can hybridise to one strand of a target nucleic acid, said reporter oligonucleotide comprising a first fluorophore, preferably in its 5′-end or within 4 nucleotides from the 5′-end, and a first quencher, preferably in its 3′-end or within 4 nucleotides from the 3′-end, wherein the reporter oligonucleotide is a sequence of in the range of 10 to 50 nucleotides into which in the range of 2 to 10 hydrophobic nucleotides have been inserted and wherein the reporter oligonucleotide comprises a hybridization sequence H, wherein
   at least one hydrophobic nucleotide is positioned at the 5′-end or within 10 nucleotides from the 5′-end of the reporter oligonucleotide; and/or
   at least one hydrophobic nucleotide is positioned at the 3′-end or within 10 nucleotides from the 3′-end of the reporter oligonucleotide; and
   wherein the hydrophobic nucleotide has the structure

X—Y-Q
wherein
   X is a nucleotide or nucleotide analogue or a backbone monomer unit capable of being incorporated into the backbone of a nucleic acid or nucleic acid analogue,
   Q is an intercalator which is not taking part in Watson-Crick hydrogen bonding; and
   Y is a linker moiety linking said nucleotide or nucleotide analogue or backbone monomer unit and said intercalator; and
   wherein the hybridisation sequence is identical to a consecutive stretch of the sequence of a first strand of the target nucleic acid, and wherein the hybridisation sequence is complementary to a consecutive stretch of the sequence of a second strand of the target nucleic acid.
37. The reporter oligonucleotide according to item 36, wherein the NOI comprises repeats, and wherein the hybridisation sequence of the reporter oligonucleotide consists of a repetitive sequence and a helper sequence in its 5′-end and/or in its 3′-end, wherein said helper sequence does not comprise repeats, and can hybridise to a second strand of the target nucleic acid when the hybridisation sequence is hybridized thereto, preferably wherein the reporter oligonucleotide consists of a repetitive sequence and only one helper sequence in its 5′-end or its 3′-end.
38. The reporter oligonucleotide according to any one of items 36 to 37, wherein the helper sequence comprises or consists of 1 to 20 nucleotides, such as 1, 2, 3, 4, 5, 6, 7, 8, 9, 10, 11, 12, 13, 14, 15, 16, 17, 18, 19 or 20 nucleotides, preferably wherein the helper sequence comprises at least one hydrophobic oligonucleotide as defined in item 1.
39. The reporter oligonucleotide according to any one of items 36 to 38, wherein the reference sequence comprises a microsatellite sequence of M tandem repeats having a total length of n nucleotides, and wherein the length of the hybridisation sequence H of the reporter oligonucleotide is n″, wherein n″≥n+1, preferably n″≥n+1 or n″≥n+2.
40. The reporter oligonucleotide according to any one of items 36 to 39, wherein the reporter oligonucleotide comprises a second quencher positioned at a non-terminal position of the reporter oligonucleotide.
41. A method of predicting the efficacy of treatment of a clinical condition in an individual in need thereof with a predetermined drug, wherein the efficacy of treatment of said clinical condition with said drug is associated with the presence of a variant sequence, said method comprising the steps of
   a. providing a sample from said individual
   b. performing the method according to any one of items 1 to 33 to determine the presence of said variant sequence;
   wherein the presence of said variant sequence is indicative of whether said drug is efficient in treating said clinical condition in said individual.
42. A method of predicting the presence of a clinical condition in an individual in need thereof, wherein said clinical condition is associated with the presence of a target nucleic acid sequence comprising a variant sequence, said method comprising the steps of
   a. providing a sample from said individual
   b. performing the method according to any one of items 1 to 33 to determine the presence of a variant sequence;
   wherein the presence of a variant sequence is indicative of said individual suffering from said clinical condition.
43. The method according to any one of items 41 to 42, wherein the clinical condition is cancer, preferably hereditary non-polyposis colorectal cancer.
44. The method according to any one of items 41 to 43, further comprising a step of administering a therapeutic agent in an effective amount to said individual.

1. A method for detecting the presence of a variant sequence in a target nucleic acid sequence consisting of two strands and comprising nucleotide(s) of interest (NOI) preferably comprising repeats, wherein said target nucleic acid sequence consists of a variant sequence or of a reference sequence, said method comprising the steps of:
a) Providing a first sample comprising nucleic acids suspected of comprising said variant sequence;
b) Providing a second sample comprising nucleic acids comprising said reference sequence, wherein the second sample is a reference sample;
c) Providing a reporter oligonucleotide;
d) Providing a set of primers consisting of a first primer and a second primer, wherein the set of primers together are capable of amplifying the target nucleic acid sequence comprising the NOI;
e) Amplifying the target nucleic acid sequence in the presence of said first sample, said first primer and said second primer, thereby obtaining a first amplicon comprising nucleic acids suspected of comprising a variant sequence; and amplifying the target nucleic acid sequence in the presence of said second sample, said first primer and said second primer, thereby obtaining a second amplicon comprising the reference sequence, wherein the second amplicon is a reference amplicon;
f) Performing melting analysis, such as high-resolution melt (HRM) analysis, of the first amplicon, thereby obtaining a first profile characterised by a first melt curve, and performing melting analysis, such as HRM analysis, of the second amplicon, thereby obtaining a second profile, characterised by a second melt curve wherein the second profile is a reference profile characterised by a reference melt curve; wherein each amplicon comprises a first strand and a second strand, wherein the melting analysis involves hybridisation of the reporter oligonucleotide to one strand of each amplicon, detection of a signal emitted by the fluorophore, and obtaining the first and the second melt curves;
wherein the reporter oligonucleotide is a sequence of in the range of 10 to 50, preferably in the range of 15 to 50 nucleotides, into which in the range of 2 to 10 hydrophobic nucleotides have been inserted,
wherein the reporter oligonucleotide comprises a first fluorophore, preferably in its 5′-end or within 4 nucleotides from the 5′-end, and a first quencher, preferably in its 3′-end or within 4 nucleotides from the 3′-end, and
wherein the reporter oligonucleotide comprises a hybridization sequence H,
wherein the hybridisation sequence is identical to a consecutive stretch of the sequence of a first strand of the target nucleic acid sequence, and wherein the hybridisation sequence is complementary to a consecutive stretch of the sequence of the second strand of the target nucleic acid sequence,
and wherein the hybridisation sequence of the reporter oligonucleotide comprises or consists of a repetitive sequence and at least one helper sequence in its 5′-end and/or in its 3′-end, wherein said helper sequence does not comprise repeats, and can hybridise to the first and second amplicons when the hybridisation sequence is hybridized thereto; and
g) Comparing the first profile to the reference profile, wherein a difference between the first profile and the reference profile indicates that the first sample contains a variant sequence.

2. The method according to claim 1, wherein step g) comprises or consists of the steps of:
i) aligning the first and the second melt curves at a given fluorescent intensity along the temperature axis, thereby nullifying differences in melting temperatures between the first and the second melt curves;
ii) determining the difference in the signal emitted by the fluorophore between the first and the second melt curves, preferably wherein the difference is a numerical difference; and
iii) comparing the difference determined in ii) to a threshold value, wherein a difference greater than the threshold indicates that the first sample comprises a variant sequence and a difference smaller than the threshold indicates that the first sample comprises the reference sequence.

3. The method according to any one of the preceding claims, wherein the reference sequence has a length of 15 nucleotides or more, such as 16, 17, 18, 19, 20, 21, 22, 23, 24, 25, 26, 27, 28, 29 or 30 nucleotides or more.

4. The method according to any one of the preceding claims, wherein the reporter oligonucleotide consists of a repetitive sequence and only one helper sequence in its 5′-end or its 3′-end or wherein the reporter oligonucleotide consists of a repetitive sequence and two helper sequences, such as one helper sequence in both the 5′-end and the 3′-end of the reporter oligonucleotide.

5. The method according to any one of the preceding claims, wherein
at least one hydrophobic nucleotide is positioned at the 5′-end or within 10 nucleotides from the 5′-end of the reporter oligonucleotide; and/or
at least one hydrophobic nucleotide is positioned at the 3′-end or within 10 nucleotides from the 3′-end of the reporter oligonucleotide; and
wherein the hydrophobic nucleotide has the structure
X—Y-Q
wherein
X is a nucleotide or nucleotide analogue or a backbone monomer unit capable of being incorporated into the backbone of a nucleic acid or nucleic acid analogue,
Q is an intercalator which is not taking part in Watson-Crick hydrogen bonding; and
Y is a linker moiety linking said nucleotide or nucleotide analogue or backbone monomer unit and said intercalator.

6. The method according to any one of the preceding claims, wherein the helper sequence comprises or consists of 1 to 20 nucleotides, such as 1, 2, 3, 4, 5, 6, 7, 8, 9, 10, 11, 12, 13, 14, 15, 16, 17, 18, 19 or 20 nucleotides.

7. The method according to any one of the preceding claims, wherein the helper sequence comprises at least one hydrophobic oligonucleotide as defined in claim 3.

8. The method according to any one of the preceding claims, wherein at least one hydrophobic nucleotide as defined in claim 3, such as 1, 2, or 3 hydrophobic nucleotides, is inserted within 1 to 10 nucleotides from the 3′-end of the reporter oligonucleotide, such as within 1, 2, 3, 4, 5, 6, 7, 8, 9 or 10 nucleotides from the 3′-end.

9. The method according to any one of the preceding claims, wherein at least one hydrophobic nucleotide as defined in claim 3, such as 1, 2, or 3 hydrophobic nucleotides, is inserted within 1 to 10 nucleotides from the 5′-end of the reporter oligonucleotide, such as within 1, 2, 3, 4, 5, 6, 7, 8, 9 or 10 nucleotides from the 5′-end.

10. The method according to any one of the preceding claims, wherein amplifying in step e) is performed by polymerase chain reaction (PCR), preferably by asymmetric PCR wherein the first and second primers are provided in different amounts, thereby directing the PCR towards amplifying more of one strand of each amplicon than of the other strand of each amplicon.

11. The method according to any one of the preceding claims, wherein the first sample has been isolated from an individual suffering from or suspected of suffering from a disease, such as a cancer, preferably the cancer is hereditary non-polyposis colorectal cancer, or from a disorder, preferably a disorder associated with microsatellite instability.

12. The method according to any one of the preceding claims, wherein the NOI is a microsatellite, such as a microsatellite sequence of M tandem repeats having a total length of n nucleotides, and wherein the variant sequence has M′ tandem repeats having a total length of n′ nucleotides, wherein M and M′ are different integers.

13. The method according to claim 12, wherein n is 15 or more, such as 16, 17, 18, 19, 20, 25, 30, 35, 40, 45, 50 or more.

14. The method according to any one of the preceding claims, wherein the length of the hybridisation sequence H of the reporter oligonucleotide is n″, wherein n″≥n+1, preferably n″≥n+1 or n″≥n+2.

15. The method according to claim 14, wherein n″ is 16 or more, such as 17, 18, 19, 20 nucleotides or more, such as 25, 30, 35, 40, 45, 50 or more.

16. The method according to any one of the preceding claims, wherein the first sample is a sample of a tissue comprising or suspected of comprising cells with mutations characteristic of said disease or disorder.

17. A kit of parts for detecting the presence of a variant sequence in a target nucleic acid sequence consisting of two strands and comprising nucleotide(s) of interest (NOI) preferably comprising repeats, wherein said target nucleic acid sequence consists of a variant sequence or of a reference sequence, said kit of parts comprising:
a) a reporter oligonucleotide comprising a first fluorophore, preferably in its 5′-end or within 4 nucleotides from the 5′-end, and a first quencher, preferably in its 3′-end or within 4 nucleotides from the 3′-end,
wherein the reporter oligonucleotide is a sequence of in the range of 10 to 50 nucleotides, preferably in the range of 15 to 50 nucleotides, into which in the range of 2 to 10 hydrophobic nucleotides have been inserted and
wherein the reporter oligonucleotide comprises a hybridization sequence H, and
wherein the hybridisation sequence is identical to a consecutive stretch of the sequence of a first strand of the target nucleic acid, and wherein the hybridisation sequence is complementary to a consecutive stretch of the sequence of a second strand of the target nucleic acid;
and wherein the hybridisation sequence of the reporter oligonucleotide comprises or consists of a repetitive sequence and at least one helper sequence in its 5′-end and/or in its 3′-end, wherein said helper sequence does not comprise repeats, and can hybridise to the first and second amplicons when the hybridisation sequence is hybridized thereto; and
b) a set of primers consisting of a first primer and a second primer, wherein the set of primers together are capable of amplifying the target nucleic acid sequence.

18. The kit according to claim 17, preferably wherein the reporter oligonucleotide, the first primer and the second primer are as defined in any one of claims 1 to 16.

19. The kit according to any one of claims 17 to 18, wherein the reporter oligonucleotide consists of a repetitive sequence and only one helper sequence in its 5′-end or its 3′-end or wherein the reporter oligonucleotide consists of a repetitive sequence and two helper sequences, such as one helper sequence in both the 5′-end and the 3′-end of the reporter oligonucleotide.

20. The kit according to any one of claims 17 to 19, wherein
at least one hydrophobic nucleotide is positioned at the 5′-end or within 10 nucleotides from the 5′-end of the reporter oligonucleotide; and/or
at least one hydrophobic nucleotide is positioned at the 3′-end or within 10 nucleotides from the 3′-end of the reporter oligonucleotide; and
wherein the hydrophobic nucleotide has the structure
X—Y-Q
wherein
X is a nucleotide or nucleotide analogue or a backbone monomer unit capable of being incorporated into the backbone of a nucleic acid or nucleic acid analogue,
Q is an intercalator which is not taking part in Watson-Crick hydrogen bonding; and
Y is a linker moiety linking said nucleotide or nucleotide analogue or backbone monomer unit and said intercalator;

21. A reporter oligonucleotide which can hybridise to one strand of a target nucleic acid consisting of two strands and comprising nucleotide(s) of interest (NOI) preferably comprising repeats, said reporter oligonucleotide comprising a first fluorophore, preferably in its 5′-end or within 4 nucleotides from the 5′-end, and a first quencher, preferably in its 3′-end or within 4 nucleotides from the 3′-end, wherein the reporter oligonucleotide is a sequence of in the range of 10 to 50, preferably in the range of 15 to 50 nucleotides, into which in the range of 2 to 10 hydrophobic nucleotides have been inserted and wherein the reporter oligonucleotide comprises a hybridization sequence H,
wherein the hybridisation sequence is identical to a consecutive stretch of the sequence of a first strand of the target nucleic acid, and wherein the hybridisation sequence is complementary to a consecutive stretch of the sequence of a second strand of the target nucleic acid,
and wherein the hybridisation sequence of the reporter oligonucleotide comprises or consists of a repetitive sequence and at least one helper sequence in its 5′-end and/or in its 3′-end, wherein said helper sequence does not comprise repeats, and can hybridise to the second strand of the first and second amplicons when the hybridisation sequence is hybridized thereto.

22. The reporter oligonucleotide according to claim 21, wherein the reporter oligonucleotide consists of a repetitive sequence and only one helper sequence in its 5′-end or its 3′-end or wherein the reporter oligonucleotide consists of a repetitive sequence and two helper sequences, such as one helper sequence in both the 5′-end and the 3′-end of the reporter oligonucleotide.

23. The reporter oligonucleotide according to any one of claims 21 to 22, wherein
at least one hydrophobic nucleotide is positioned at the 5′-end or within 10 nucleotides from the 5′-end of the reporter oligonucleotide; and/or
at least one hydrophobic nucleotide is positioned at the 3′-end or within 10 nucleotides from the 3′-end of the reporter oligonucleotide; and
wherein the hydrophobic nucleotide has the structure
X—Y-Q
wherein
X is a nucleotide or nucleotide analogue or a backbone monomer unit capable of being incorporated into the backbone of a nucleic acid or nucleic acid analogue,
Q is an intercalator which is not taking part in Watson-Crick hydrogen bonding; and
Y is a linker moiety linking said nucleotide or nucleotide analogue or backbone monomer unit and said intercalator.

24. The reporter oligonucleotide according to any one of claims 21 to 14, wherein the helper sequence comprises or consists of 1 to 20 nucleotides, such as 1, 2, 3, 4, 5, 6, 7, 8, 9, 10, 11, 12, 13, 14, 15, 16, 17, 18, 19 or 20 nucleotides, preferably wherein the helper sequence comprises at least one hydrophobic oligonucleotide as defined in claim 3.

25. A method of predicting the efficacy of treatment of a clinical condition in an individual in need thereof with a predetermined drug, wherein the efficacy of treatment of said clinical condition with said drug is associated with the presence of a variant sequence, said method comprising the steps of
a. providing a sample from said individual
b. performing the method according to any one of claims 1 to 16 to determine the presence of said variant sequence;
wherein the presence of said variant sequence is indicative of whether said drug is efficient in treating said clinical condition in said individual.

26. A method of predicting the presence of a clinical condition in an individual in need thereof, wherein said clinical condition is associated with the presence of a target nucleic acid sequence comprising a variant sequence, said method comprising the steps of
a. providing a sample from said individual
b. performing the method according to any one of claims 1 to 16 to determine the presence of a variant sequence;
wherein the presence of a variant sequence is indicative of said individual suffering from said clinical condition.
